SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2026/0297066 A1
(43) Pub. Date: Oct. 1, 2026

(54) COMPOUNDS FOR TREATING CANCER

(71) Applicant: Genesis Molecular AI, Inc., Burlingame, CA (US)

(72) Inventors: Brett Busch, San Diego, CA (US); Brandon Reinus, San Diego, CA (US); William F. Vernier, Vista, CA (US); Martin Indarte, Krakow (PL)

(21) Appl. No.: 19/480,254

(22) PCT Filed: May 1, 2024
(86) PCT No.: PCT/US2024/027254

§ 371 Date: Oct. 30, 2025

Related U.S. Application Data
(60) Provisional application No. 63/463,353, filed on May 2, 2023.

(51) Int. Cl. C07D 403/14; A61K 31/4035; A61K 31/422; A61K 31/437; A61K 31/4439; A61K 31/444; A61K 31/454; A61K 31/4709; A61K 31/501; A61K 31/517; A61K 31/55; A61P 35/00; C07D 209/46; C07D 401/04; C07D 401/06; C07D 401/08; C07D 401/12; C07D 403/04; C07D 405/04; C07D 405/08; C07D 405/10; C07D 405/12; C07D 405/14; C07D 413/14; C07D 471/04; C07D 493/10

(52) U.S. Cl. CPC C07D 403/14; A61K 31/4035; A61K 31/422; A61K 31/437; A61K 31/4439; A61K 31/444; A61K 31/454; A61K 31/4709; A61K 31/501; A61K 31/517; A61K 31/55; A61P 35/00; C07D 209/46; C07D 401/04; C07D 401/06; C07D 401/08; C07D 401/12; C07D 403/04; C07D 405/04; C07D 405/08; C07D 405/10; C07D 405/12; C07D 405/14; C07D 413/14; C07D 471/04; C07D 493/10

(57) ABSTRACT

This disclosure provides compounds of Formula (I) and pharmaceutically acceptable salts thereof, that inhibit phosphatidylinositol 4,5-bisphosphate 3-kinase (PI3K) isoform alpha (PI3Kα).

### CROSS-REFERENCE TO RELATED APPLICATIONS

This application claims the benefit of priority to U.S. Application No. 63/463,353 filed on May 2, 2023, the contents of which are hereby incorporated by reference.

### TECHNICAL FIELD

This disclosure provides compounds of Formula (I) and pharmaceutically acceptable salts thereof, that inhibit phosphatidylinositol 4,5-bisphosphate 3-kinase (PI3K) isoform alpha (PI3Kα).

### BACKGROUND

Phosphatidylinositol 4,5-bisphosphate 3-kinase (PI3K) isoform alpha (PI3Kα), encoded by the PIK3CA gene is a part of the PI3K/AKT/TOR signaling network and is altered in several human cancers.

Activation of the PI3K pathway occurs in approximately 30-50% human cancers and contributes to resistance to various anti-cancer therapies. (See, Bauer, T. M. et al., Pharmacol. Ther. 2015, 146, 53-60.) However, development of PI3K inhibitors has been problematic for several reasons, in particular, inability to specifically inhibit signaling by mutant PI3Kα while sparing wild-type PI3Kα, and the related dose-limiting toxicities that prevent sustained PI3K pathway suppression. (See, Hanker et al., Cancer Discovery, April 2019; 9: 482-491.)

Thus, selectively targeting PI3Kα represents an approach for the treatment of proliferative disorders such as cancer.

### SUMMARY

Some embodiments provide a compound of Formula (I):

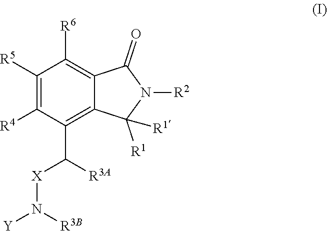

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1′} is hydrogen or C1-C6 alkyl; or R¹ and R^{1′}, together with the carbon atom to which they are attached form a C3-C10 cycloalkyl;
R² is C6-C12 aryl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, C4-C10 cycloalkyl optionally substituted with 1-4 independently selected R^{2A}, C1-C6 alkoxyalkyl optionally substituted with —C(═O)NR^{A}R^{C}, C1-C6 aralkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

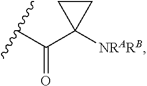

(vii) —NHC(═O)R^{C},
(viii) —C(═O)R^{D},
(ix) —C(═O)OR^{E},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl, —C(═O)NR^{A}R^{B}, C1-C6 haloalkyl, and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl,
(xx) C3-C6 cycloalkyl optionally substituted with 4-10 membered heterocyclyl optionally substituted with C1-C6 alkyl;
(xxi) C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}; and
(xxii) 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B};
each R^{A} and R^{B} is independently selected from hydrogen, hydroxyl, C1-C6 alkoxy, C3-C6 cycloalkyl, C2-C6 alkenyl, cyano, 4-10 membered heterocyclyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, hydroxyl, C1-C6 alkyl, and —C(═O)C1-C6 alkyl;
each R^{C} is independently selected from C3-C6 cycloalkyl, —C(═O)NHR^{Y1}, C2-C6 alkenyl, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, phenyl, 4-10 membered heterocyclyl, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, halogen, C1-C6 haloalkyl, C3-C6 cycloalkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

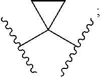 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR¹, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Also provided herein is a pharmaceutical composition comprising a compound of Formula (I), or a pharmaceutically acceptable salt thereof, and one or more pharmaceutically acceptable excipients.

Provided herein is a method for treating cancer in a subject in need thereof, comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, comprising (a) determining that the cancer is associated with a dysregulation of a PIK3CA gene, a PI3Kα protein, or expression or activity or level of any of the same; and (b) administering to the subject a therapeutically effective amount of a compound of Formula (I), or a pharmaceutically acceptable salt thereof, or a pharmaceutical composition as provided herein.

Provided herein is a method of treating a PI3Kα-associated disease in a subject, comprising administering to a subject identified or diagnosed as having a PI3Kα-associated disease a therapeutically effective amount of a compound of Formula (I), or a pharmaceutically acceptable salt thereof, or a pharmaceutical composition as provided herein.

This disclosure also provides a method of treating a PI3Kα-associated disease in a subject, comprising: determining that the cancer in the subject is a PI3Kα-associated disease; and administering to the subject a therapeutically effective amount of a compound of Formula (I), or a pharmaceutically acceptable salt thereof, or a pharmaceutical composition as provided herein.

Further provided herein is a method of treating a PI3Kα-associated cancer in a subject, comprising administering to a subject identified or diagnosed as having a PI3Kα-associated cancer a therapeutically effective amount of a compound of Formula (I), or a pharmaceutically acceptable salt thereof, or a pharmaceutical composition as provided herein.

This disclosure also provides a method of treating a PI3Kα-associated cancer in a subject, comprising: determining that the cancer in the subject is a PI3Kα-associated cancer; and administering to the subject a therapeutically effective amount of a compound of Formula (I), or a pharmaceutically acceptable salt thereof, or a pharmaceutical composition as provided herein.

Provided herein is a method of treating a subject, comprising administering a therapeutically effective amount of a compound of Formula (I), or a pharmaceutically acceptable salt thereof, or a pharmaceutical composition as provided herein, to a subject having a clinical record that indicates that the subject has a dysregulation of a PIK3CA gene, a PI3Kα protein, or expression or activity or level of any of the same.

This disclosure also provides a method for inhibiting PI3Kα in a mammalian cell, comprising contacting the mammalian cell with an effective amount of a compound of Formula (I), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The phrase “therapeutically effective amount” means an amount of compound that, when administered to a subject in need of such treatment, is sufficient to (i) treat a PI3Kα protein-associated disease, (ii) attenuate, ameliorate, or eliminate one or more symptoms of the particular disease, or (iii) delay the onset of one or more symptoms of the particular disease described herein.

The term “pharmaceutically acceptable salt” refers to a formulation of a compound that does not cause significant irritation to an organism to which it is administered and does not abrogate the biological activity and properties of the compound.

As used herein, the term “subject” refer to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease to be treated and/or prevented.

As used herein, terms “treat” or “treatment” refer to therapeutic or palliative measures. Beneficial or desired clinical results include, but are not limited to, alleviation, in whole or in part, of symptoms associated with a disease, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halogen.

The term “alkoxy” refers to an —O-alkyl radical (e.g., —OCH₃).

The term “alkoxyalkyl” refers to an -alkyl-O-alkyl radical (e.g., —CH₂CH₂OCH₃).

The term “thioalkyl” refers to an —S-alkyl radical (e.g., —SCH₃) or an -alkyl-S-alkyl radical (e.g., —CH₂CH₂SCH₃).

The term “hydroxyalkyl” refers to an alkyl, in which one or more hydrogen atoms is/are replaced with hydroxyl.

The term “aryl” refers to a 6-20 membered all carbon ring system wherein at least one ring in the system is aromatic (e.g., 6-carbon monocyclic, 10-carbon bicyclic, or 14-carbon tricyclic aromatic ring system). Examples of aryl groups include phenyl, naphthyl, tetrahydronaphthyl, 2,3-dihydro-1H-indene, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1. 1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, refers to a ring system having 5 to 20 ring atoms, such as 5, 6, 9, 10, or 14 ring atoms; wherein at least one ring in the system contains one or more heteroatoms independently selected from the group consisting of N, O, S, Si, and B, and at least one ring in the system is aromatic (but does not have to be a ring which contains a heteroatom, e.g. tetrahydroisoquinolinyl, e.g., tetrahydroquinolinyl). Heteroaryl groups can include monocyclic, bridged, fused, and spiro ring systems, so long as one ring in the system is aromatic. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

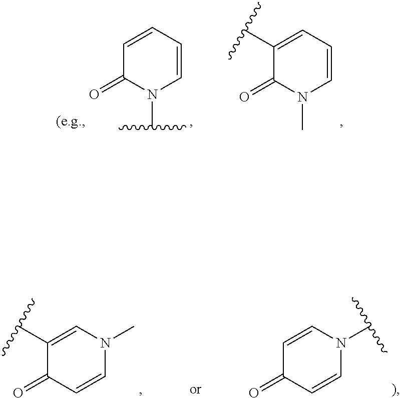

pyrimidone

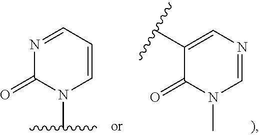

pyridazinone

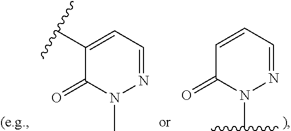

pyrazinone

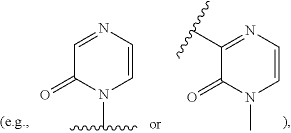

and imidazolone

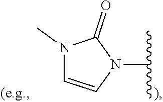 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a saturated or partially unsaturated ring systems with 3-16 ring atoms (e.g., 3-8 membered monocyclic, 5-12 membered bicyclic, or 10-14 membered tricyclic ring system) having at least one heteroatom selected from O, N, S, Si, and B, wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting. Heterocyclyl groups include monocyclic, bridged, fused, and spiro ring systems. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

A “heterocyclyloxy” refers to an —O-heterocyclyl radical

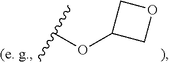 [SMILES: *OC1COC1]

An “aralkyl” refers to an aryl group, as defined herein, connected to the remainder of the molecule via a divalent C1-C6 alkyl group, as described herein. Non-limiting examples of an aralkyl group are benzyl, ethylphenyl, methylnaphthyl, and the like.

A “heteroaralkyl” refers to a heteroaryl group, as defined herein, connected to the remainder of the molecule via a divalent C1-C6 alkyl group, as described herein. Non-limiting examples of an aralkyl group are methylpyridyl, ethylpyrimidinyl, methylimidazolyl, and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

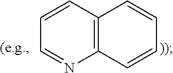

(ii) a single ring atom (spiro-fused ring systems)

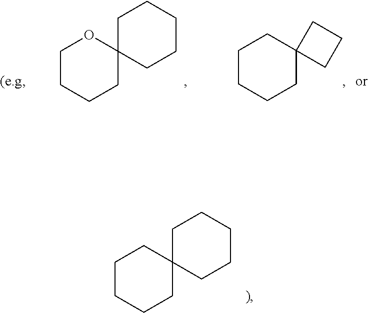 [SMILES: C1CCC2(CC1)CCCCO2 ; C1CCC2(CC1)CCC2 ; C1CCC2(CC1)CCCCC2]

or (iii) a contiguous array of ring atoms (bridged ring systems having all bridge lengths >0)

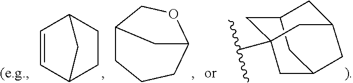

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

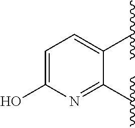 [SMILES: *c1ccc(O)nc1*]

encompasses the tautomeric form containing the moiety:

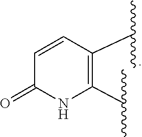 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

The details of one or more embodiments of this disclosure are set forth in the accompanying drawings and the description below. Other features and advantages of the present disclosure will be apparent from the description and drawings, and from the claims.

### DETAILED DESCRIPTION

This disclosure provides compounds of Formula (I) and pharmaceutically acceptable salts thereof, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### Formulae (I) Compounds

Some embodiments provide a compound of Formula (I):

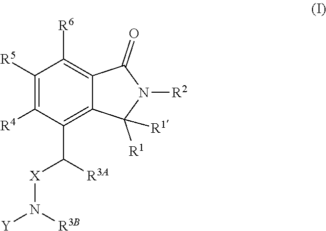

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1′} is hydrogen or C1-C6 alkyl; or R¹ and R^{1′}, together with the carbon atom to which they are attached form a C3-C10 cycloalkyl;
R² is C6-C12 aryl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R²¹, C4-C10 cycloalkyl optionally substituted with 1-4 independently selected R^{2A}, C1-C6 alkoxyalkyl optionally substituted with —C(═O)NR^{A}R^{C}, C1-C6 aralkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

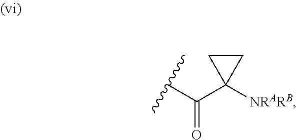

(vii) —NHC(═O)R^{C},
(viii) —C(═O)R^{D},
(ix) —C(═O)OR^{E},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl, C1-C6 haloalkyl, —C(═O)NR^{A}R^{B}, and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl,
(xx) C3-C6 cycloalkyl optionally substituted with 4-10 membered heterocyclyl optionally substituted with C1-C6 alkyl;
(xxi) C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}; and
(xxii) 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B};
each R^{A} and R^{B} is independently selected from hydrogen, hydroxyl, C1-C6 alkoxy, C3-C6 cycloalkyl, C2-C6 alkenyl, cyano, 4-10 membered heterocyclyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, hydroxyl, C1-C6 alkyl, and —C(═O)C1-C6 alkyl;
each R^{C} is independently selected from C3-C6 cycloalkyl, —C(═O)NHR^{Y1}, C2-C6 alkenyl, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, phenyl, 4-10 membered heterocyclyl, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, halogen, C1-C6 haloalkyl, C3-C6 cycloalkyl, cyano, —NR^{5A}R^{5B}B, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

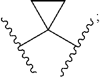 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, the compound of Formula (I) or a pharmaceutically acceptable salt thereof, is a compound of Formula (I-A):

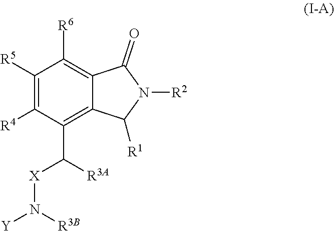

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is aryl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, C4-C10 cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

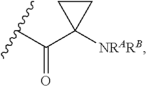

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, cyano, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, C2-C6 alkenyl, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group,
R⁴ is hydrogen, C1-C6 alkyl, or acrylamido;
R⁵ is hydrogen, C1-C6 alkyl, halogen, C1-C6 haloalkyl, C3-C6 cycloalkyl, cyano, —NR^{5A}R^{5B}B, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

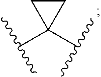 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(CJ-C6 alkyl) or CJ-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, R¹ is hydrogen.

In some embodiments, R¹ is cyano.

In some embodiments, R¹ is C3-C6 cycloalkyl. In some embodiments, R¹ is cyclopropyl or cyclobutyl.

In some embodiments, R¹ is C1-C6 alkyl optionally substituted with phenyl optionally substituted with halogen. In some embodiments, R¹ is C1-C6 alkyl substituted with phenyl optionally substituted with halogen. In some embodiments, R¹ is C1-C6 alkyl substituted with phenyl substituted with halogen. In some embodiments, R¹ is para-fluorobenzyl. In some embodiments, R¹ is C1-C6 alkyl substituted with phenyl. In some embodiments, R¹ is benzyl. In some embodiments, R¹ is ethyl-1-phenyl or ethyl-2-phenyl. In some embodiments, R¹ is C1-C6 alkyl. In some embodiments, R¹ is methyl, ethyl, or isopropyl. In some embodiments, R¹ is methyl.

In some embodiments, R¹ is C1-C6 thioalkyl. In some embodiments, R¹ is C1-C3 thioalkyl. In some embodiments, R¹ is thiomethyl, thioethyl, or thiopropyl. In some embodiments, R¹ is methyl-thiomethyl, methyl-thioethyl, or ethyl-thiomethyl. In some embodiments, R¹ is thiomethyl.

In some embodiments, R¹ is C1-C6 haloalkyl. In some embodiments, R¹ is CT-C3 haloalkyl. In some embodiments, R¹ is CJ-C3 fluoroalkyl. In some embodiments, R¹ is CF₃. In some embodiments, R¹ is CHF₂.

In some embodiments, R¹ is C1-C6 alkoxy. In some embodiments, R¹ is C1-C3 alkoxy. In some embodiments, R¹ is —OCH₃, —OCH₂CH₃, or —OCH₂CH₂CH₃. In some embodiments, R¹ is —OCH₃.

In some embodiments, R¹ is CJ-C6 alkoxyalkyl. In some embodiments, R¹ is C1-C3 alkoxyalkyl. In some embodiments, R¹ is —CH₂OCH₃, —CH₂OCH₂CH₃, or —CH₂CH₂OCH₃. In some embodiments, R¹ is —CH₂OCH₃.

In some embodiments, R^{1′} is hydrogen.

In some embodiments, R^{1′} is C1-C6 alkyl. In some embodiments, R^{1′} is methyl.

In some embodiments, R¹ and R^{1′}, together with the carbon atom to which they are attached form a C3-C10 cycloalkyl. In some embodiments, R¹ and R^{1′}, together with the carbon atom to which they are attached form a cyclopropyl.

In some embodiments, R² is C6-C12 aryl optionally substituted with 1-4 independently selected R^{2A}. In some embodiments, R² is phenyl optionally substituted with 1-4 independently selected R^{2A}. In some embodiments, R² is phenyl substituted with 1 or 2 independently selected R^{2A}. In some embodiments, R² is phenyl substituted with 1 R^{2A}. In some embodiments, R² is phenyl substituted with 2 independently selected R^{2A}. In some embodiments, R² is phenyl optionally substituted with 3 independently selected R^{2A}. In some embodiments, R² is phenyl.

In some embodiments, R² is 2,3-dihydro-1H-indenyl optionally substituted with 1-4 independently selected R^{2A}. In some embodiments, R² is 2,3-dihydro-1H-indenyl substituted with 1 or 2 independently selected R^{2A}. In some embodiments, R² is 2,3-dihydro-1H-indenyl substituted with 1 R^{2A}. In some embodiments, R² is 2,3-dihydro-1H-indenyl substituted with 2 independently selected R^{2A}. In some embodiments, R² is 2,3-dihydro-1H-indenyl optionally substituted with 3 independently selected R^{2A}. In some embodiments, R² is 2,3-dihydro-1H-indenyl.

In some embodiments, R² is 5-10 membered heteroaryl optionally substituted with 1-4 independently selected R^{2A}. In some embodiments, R² is 5-10 membered heteroaryl substituted with 1 or 2 independently selected R^{2A}. In some embodiments, R² is 5-10 membered heteroaryl substituted with 1 R^{2A}. In some embodiments, R² is 5-10 membered heteroaryl substituted with 2 independently selected R^{2A}. In some embodiments, R² is 5-10 membered heteroaryl optionally substituted with 3 independently selected R^{2A}. In some embodiments, R² is 5-10 membered heteroaryl.

In some embodiments, R² is 6 membered heteroaryl optionally substituted with 1-4 independently selected R^{2A}. In some embodiments, R² is 6 membered heteroaryl substituted with 1 or 2 independently selected R^{2A}. In some embodiments, R² is 6 membered heteroaryl substituted with 1 R^{2A}. In some embodiments, R² is 6 membered heteroaryl substituted with 2 independently selected R^{2A}. In some embodiments, R² is 6 membered heteroaryl optionally substituted with 3 independently selected R^{2A}. In some embodiments, R² is 6 membered heteroaryl.

In some embodiments, R² is 9 membered heteroaryl optionally substituted with 1-4 independently selected R^{2A}. In some embodiments, R² is 9 membered heteroaryl substituted with 1 or 2 independently selected R^{2A}. In some embodiments, R² is 9 membered heteroaryl substituted with 1 R^{2A}. In some embodiments, R² is 9 membered heteroaryl substituted with 2 independently selected R^{2A}. In some embodiments, R² is 9 membered heteroaryl optionally substituted with 3 independently selected R^{2A}. In some embodiments, R² is 9 membered heteroaryl.

In some embodiments, the heteroaryl of R² is pyridinyl, pyrimidinyl, pyridazinyl, indole, indazole, azaindole, azaindazole, indoline, azaindoline, isoindoline, azaisoindoline, benzimidazolyl, benzoxazolyl, benzothiazolyl, benzoisoxazolyl, benzisothiazolyl, quinolinyl, 6,7-dihydro-5H-cyclopenta[c]pyridinyl, 6,7-dihydro-5H-cyclopenta[b]pyridinyl, chromanyl, 3,4-dihydro-2H-1l2-quinolinyl, 5,6,7,8-tetrahydroquinazolinyl, 6,7-dihydro-5H-cyclopenta[c]pyridinyl, 3,4-dihydro-2H-pyrano[2,3-b]pyridinyl, 3,4-dihydro-2H-pyrano[2,3-c]pyridinyl, 3,4-dihydro-2H-pyrano[3,2-b]pyridinyl, 7,8-dihydro-6H-pyrano[3,2-d]pyrimidinyl, 5,6,7,8-tetrahydroquinazolinyl, or isoquinolinyl. In some embodiments, the heteroaryl of R² is pyridinyl or pyrimidinyl. In some embodiments, the heteroaryl of R² is indole, indazole, azaindole, azaindazole, indoline, azaindoline, isoindoline, or azaisoindoline. In some embodiments, the heteroaryl of R² is 6,7-dihydro-5H-cyclopenta[c]pyridinyl, 6,7-dihydro-5H-cyclopenta[b]pyridinyl, chromanyl, 3,4-dihydro-2H-1l2-quinolinyl, 5,6,7,8-tetrahydroquinazolinyl, 6,7-dihydro-5H-cyclopenta[c]pyridinyl, 3,4-dihydro-2H-pyrano[2,3-b]pyridinyl, 3,4-dihydro-2H-pyrano[2,3-c]pyridinyl, 3,4-dihydro-2H-pyrano[3,2-b]pyridinyl, 7,8-dihydro-6H-pyrano[3,2-d]pyrimidinyl, or 5,6,7,8-tetrahydroquinazolinyl. In some embodiments, the heteroaryl of R² is 6,7-dihydro-5H-cyclopenta[c]pyridinyl. In some embodiments, the heteroaryl of R² is 6,7-dihydro-5H-cyclopenta[b]pyridinyl. In some embodiments, the heteroaryl of R² is chromanyl. In some embodiments, the heteroaryl of R² is 3,4-dihydro-2H-1l2-quinolinyl, 5,6,7,8-tetrahydroquinazolinyl, or 6,7-dihydro-5H-cyclopenta[c]pyridinyl. In some embodiments, the heteroaryl of R² is 3,4-dihydro-2H-1l2-quinolinyl. In some embodiments, the heteroaryl of R² is 5,6,7,8-tetrahydroquinazolinyl. In some embodiments, the heteroaryl of R² is 6,7-dihydro-5H-cyclopenta[c]pyridinyl. In some embodiments, the heteroaryl of R² is 4-dihydro-2H-pyrano[2,3-b]pyridinyl. In some embodiments, the heteroaryl of R² is 3,4-dihydro-2H-pyrano[2,3-c]pyridinyl, or 5,6,7,8-tetrahydroquinazolinyl. In some embodiments, the heteroaryl of R² is 3,4-dihydro-2H-pyrano[3,2-b]pyridinyl. In some embodiments, the heteroaryl of R² is 7,8-dihydro-6H-pyrano[3,2-d]pyrimidinyl. In some embodiments, the heteroaryl of R² is 5,6,7,8-tetrahydroquinazolinyl.

In some embodiments, R² is 4-10 membered heterocyclyl optionally substituted with 1-4 independently selected R^{2A}. In some embodiments, R² is 4-10 membered heterocyclyl substituted with 1 or 2 independently selected R^{2A}. In some embodiments, R² is 4-10 membered heterocyclyl substituted with 1 R^{2A}. In some embodiments, R² is 4-10 membered heterocyclyl substituted with 2 independently selected R^{2A}. In some embodiments, R² is 4-10 membered heterocyclyl optionally substituted with 3 independently selected R^{2A}. In some embodiments, R² is 4-10 membered heterocyclyl.

In some embodiments, R² is 5-8 membered heterocyclyl optionally substituted with 1-4 independently selected R^{2A}. In some embodiments, R² is 5-8 membered heterocyclyl substituted with 1 or 2 independently selected R^{2A}. In some embodiments, R² is 5-8 membered heterocyclyl substituted with 1 R^{2A}. In some embodiments, R² is 5-8 membered heterocyclyl substituted with 2 independently selected R^{2A}. In some embodiments, R² is 5-8 membered heterocyclyl optionally substituted with 3 independently selected R^{2A}. In some embodiments, R² is 5-8 membered heterocyclyl.

In some embodiments, the heterocyclyl of R² is piperidinyl, piperazinyl, octahydrocyclopenta[c]pyranyl, octahydrocyclopenta[b]pyranyl, 2-oxabicyclo[2.1.1]hexanyl, 2-oxabicyclo[3.1.1]heptanyl, or morpholinyl. In some embodiments, the heterocyclyl of R² is octahydrocyclopenta[c]pyranyl. In some embodiments, the heterocyclyl of R² is octahydrocyclopenta[b]pyranyl. In some embodiments, the heterocyclyl of R² is 2-oxabicyclo[2.1.1]hexanyl. In some embodiments, the heterocyclyl of R² is 2-oxabicyclo[3.1. 1]heptanyl.

In some embodiments, R² is C4-C10 cycloalkyl optionally substituted with 1-4 independently selected R^{2A}. In some embodiments, R² is C4-C10 cycloalkyl substituted with 1 or 2 independently selected R^{2A}. In some embodiments, R² is C4-C10 cycloalkyl substituted with 1 R^{2A}. In some embodiments, R² is C4-C10 cycloalkyl substituted with 2 independently selected R^{2A}. In some embodiments, R² is C4-C10 cycloalkyl optionally substituted with 3 independently selected R^{2A}. In some embodiments, R² is C4-C10 cycloalkyl.

In some embodiments, R² is C5-C7 cycloalkyl optionally substituted with 1-4 independently selected R^{2A}. In some embodiments, R² is C5-C7 cycloalkyl substituted with 1 or 2 independently selected R^{2A}. In some embodiments, R² is 5C5-C7 cycloalkyl substituted with 1 R^{2A}. In some embodiments, R² is C5-C7 cycloalkyl substituted with 2 independently selected R^{2A}. In some embodiments, R² is C5-C7 cycloalkyl optionally substituted with 3 independently selected R^{2A}. In some embodiments, R² is C5-C7 cycloalkyl.

In some embodiments, the cycloalkyl of R² is cyclopentyl, [1.1.1]bicyclopentyl, octahydro-1H-indenyl, bicyclo[2.1.1]hexanyl, bicyclo[3.1.0]hexanyl, spiro[2.4]heptanyl, or cyclohexyl. In some embodiments, the cycloalkyl of R² is octahydro-1H-indenyl. In some embodiments, the cycloalkyl of R² is bicyclo[2.1.1]hexanyl. In some embodiments, the cycloalkyl of R² is bicyclo[3.1.0]hexanyl.

In some embodiments, R² is C1-C6 aralkyl optionally substituted with 1-4 independently selected R^{2A}. In some embodiments, R² is C1-C6 aralkyl. In some embodiments, R² is C1-C6 aralkyl substituted with 1-4 independently selected R^{2A}. In some embodiments, R² is benzyl substituted with 1-4 independently selected R^{2A}.

In some embodiments, R² is C1-C6 alkoxyalkyl optionally substituted with —C(═O)NR^{A}R^{C}. In some embodiments, R² is C1-C6 alkoxyalkyl. In some embodiments, R² is C1-C3 alkoxyalkyl. In some embodiments, R² is —CH₂OCH₃, —CH₂OCH₂CH₃, or —CH₂CH₂OCH₃. In some embodiments, R² is —CH₂OCH₃.

In some embodiments, 1, 2, 3, or 4 of R^{2A} are independently halogen. In some embodiments, 1, 2, or 3 of R^{2A} are independently fluoro or chloro. In some embodiments, 1 or 2 of R^{2A} are independently fluoro or chloro.

In some embodiments, 1, 2, 3, or 4 of R^{2A} are independently cyano. In some embodiments, 1 or 2 of R^{2A} are cyano.

In some embodiments, 1, 2, 3, or 4 of R^{2A} are independently hydroxyl. In some embodiments, 1 or 2 of R^{2A} are hydroxyl.

In some embodiments, 1, 2, 3, or 4 of R^{2A} are independently —NR^{A}R^{B}. In some embodiments, 1 or 2 of R^{2A} are independently —NR^{A}R^{B}.

In some embodiments, 1, 2, 3, or 4 of R^{2A} are independently —C(═O)NR^{A}R^{B}. In some embodiments, 1 or 2 of R^{2A} are independently —C(═O)NR^{A}R^{B}.

In some embodiments, 1, 2, 3, or 4 of R^{2A} are independently

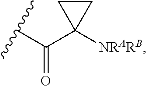

In some embodiments, 1 or 2 of R^{2A} are independently

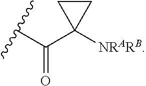

In some embodiments, 1, 2, 3, or 4 of R^{2A} are independently —NHC(═O)R^{C}. In some embodiments, 1 or 2 of R^{2A} are independently —NHC(═O)R^{C}.

In some embodiments, 1, 2, 3, or 4 of R^{2A} are independently —C(═O)R^{D}. In some embodiments, 1 or 2 of R^{2A} are independently —C(═O)R^{D}.

In some embodiments, 1, 2, 3, or 4 of R^{2A} are independently —C(═O)OR^{F}. In some embodiments, 1 or 2 of R^{2A} are independently —C(═O)OR^{E}.

In some embodiments, 1, 2, 3, or 4 of R^{2A} are independently —SO₂R^{F}. In some embodiments, 1 or 2 of R^{2A} are independently —SO₂R^{F}.

In some embodiments, 1, 2, 3, or 4 of R^{2A} are independently —NHSO₂R^{F}. In some embodiments, 1 or 2 of R^{2A} are independently —NHSO₂R^{F}.

In some embodiments, 1, 2, 3, or 4 of R^{2A} are independently —SO₂NR^{F}R^{G}. In some embodiments, 1 or 2 of R^{2A} are independently —SO₂NR^{F}R^{G}.

In some embodiments, 1, 2, 3, or 4 of R^{2A} are independently —NHC(═O)C1-C6 alkyl optionally substituted with NR^{A}R^{B}. In some embodiments, 1, 2, or 3 of R^{2A} are independently —NHC(═O)C1-C6 alkyl substituted with NR^{A}R^{B}. In some embodiments, 1, 2, or 3 of R^{2A} are independently —NHC(═O)C1-C6 alkyl. In some embodiments, 1 or 2 of R^{2A} are independently —NHC(═O)C1-C6 alkyl optionally substituted with NR^{A}R^{B}. In some embodiments, 1 or 2 of R^{2A} are independently —NHC(═O)C1-C6 alkyl substituted with NR^{A}R^{B}. In some embodiments, 1 or 2 of R^{2A} are independently —NHC(═O)C1-C6 alkyl.

In some embodiments, 1, 2, 3, or 4 of R^{2A} are independently C1-C6 haloalkyl. In some embodiments, 1 or 2 of R^{2A} are independently C1-C3 haloalkyl. In some embodiments, 1 or 2 of R^{2A} are trifluoromethyl.

In some embodiments, 1, 2, 3, or 4 of R^{2A} are independently C1-C6 hydroxyalkyl. In some embodiments, 1 or 2 of R^{2A} are independently C1-C3 hydroxyalkyl.

In some embodiments, 1, 2, 3, or 4 of R^{2A} are independently 5-10 membered heteroaryl optionally substituted with 1-3 substituents independently selected from C1-C6 alkyl, —C(═O)NR^{A}R^{B}, C1-C6 haloalkyl, and —NR^{A}R^{B}. In some embodiments, 1, 2, 3, or 4 of R^{2A} are independently 5-10 membered heteroaryl substituted with 1-3 substituents independently selected from C1-C6 alkyl, —C(═O)NR^{A}R^{B}, C1-C6 haloalkyl, and —NR^{A}R^{B}. In some embodiments, 1, 2, 3, or 4 of R^{2A} are independently 5-10 membered heteroaryl.

In some embodiments, 1 of R^{2A} is 5-6 membered heteroaryl optionally substituted with 1-3 substituents independently selected from C1-C6 alkyl, —C(═O)NR^{A}R^{B}, C1-C6 haloalkyl, and —NR^{A}R^{B}. In some embodiments, 1 of R^{2A} is 5-6 membered heteroaryl substituted with 1-3 substituents independently selected from C1-C6 alkyl, —C(═O)NR^{A}R^{B}, C1-C6 haloalkyl, and —NR^{A}R^{B}. In some embodiments, 1 of R^{2A} is 5-6 membered heteroaryl.

In some embodiments, 1, 2, 3, or 4 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, 1 of R^{2A} is 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl.

In some embodiments, 1, 2, 3, or 4 of R^{2A} are independently 4-10 membered heterocyclyl. In some embodiments, 1 of R^{2A} is 4-10 membered heterocyclyl.

In some embodiments, 1, 2, or 3 of R^{2A} are independently C1-C6 alkyl optionally substituted with 1-3 substituents independently selected from hydroxyl, oxo, —NR^{A}R^{B}, —C(═O)NR^{A}R^{B}, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B} C3-C10 cycloalkyl, phenyl, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C3-C1 cycloalkyl, phenyl, and 4-10 membered heterocyclyl.

In some embodiments, 1, 2, 3, or 4 of R^{2A} are independently C1-C6 alkyl. In some embodiments, 1, 2, or 3 of R^{2A} are independently C1-C3 alkyl. In some embodiments, 1, 2, or 3 of R^{2A} are methyl. In some embodiments, 1 or 2 of R^{2A} are independently C1-C6 alkyl. In some embodiments, 1 or 2 of R^{2A} are independently C1-C3 alkyl. In some embodiments, 1 or 2 of R^{2A} are methyl.

In some embodiments, 1, 2, 3, or 4 of R^{2A} are independently C1-C6 alkoxy optionally substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl substituted with C1-C6 alkyl, hydroxyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl.

In some embodiments, 1, 2, 3, or 4 of R^{2A} are independently C1-C6 alkoxy.

In some embodiments, 1, 2, 3, or 4 of R^{2A} are independently C3-C6 cycloalkyl optionally substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl substituted with C1-C6 alkyl.

In some embodiments, 1, 2, 3, or 4 of R^{2A} are independently C3-C6 cycloalkyl substituted with 4-10 membered heterocyclyl.

In some embodiments, 1, 2, 3, or 4 of R^{2A} are independently C3-C6 cycloalkyl. In some embodiments, 1 or 2 of R^{2A} are independently C3-C6 cycloalkyl.

In some embodiments, 1, 2, 3, or 4 of R^{2A} are independently C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}. In some embodiments, 1 of R^{2A} is C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}.

In some embodiments, 1, 2, 3, or 4 of R^{2A} are independently 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, 1 of R^{2A} is 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B}.

In some embodiments, R² is substituted with 1 R^{2A}. In some embodiments, R² is substituted with 2 independently selected R^{2A}. In some embodiments, R² is substituted with 3 independently selected R^{2A}. In some embodiments, R² is substituted with 4 independently selected R^{2A}.

In some embodiments, R² is piperidinyl substituted with 1-2 independently selected R^{2A}In some embodiments, R² is piperidinyl substituted with 2-4 independently selected R^{2A}. In some embodiments, R² is morpholinyl substituted with 2-4 independently selected R^{2A}. In some embodiments, R² is morpholinyl. In some embodiments, R² is piperazinyl substituted with 1-2 independently selected R^{2A}.

In some embodiments, R² is phenyl substituted with 1-2 independently selected R^{2A}. In some embodiments, R² is pyridinyl substituted with 1-2 independently selected R^{2A}.

In some embodiments, R² is indolyl substituted with 1-2 independently selected R^{2A}. In some embodiments, R² is indazolyl substituted with 1-2 independently selected R^{2A}. In some embodiments, R² is 7-azaindolyl substituted with 1-2 independently selected R^{2A}. In some embodiments, R² is 7-azaindazolyl substituted with 1-2 independently selected R^{2A}. In some embodiments, R² is 1,2-dihydro-3H-indazol-3-one substituted with 1-2 independently selected R^{2A}. In some embodiments, R² is isoindolinyl substituted with 1-2 independently selected R^{2A}. In some embodiments, R² is isoindolinyl. In some embodiments, R² is 2-indolinone substituted with 1-2 independently selected R^{2A}. In some embodiments, R² is benzimidazolyl substituted with 1-2 independently selected R^{2A}. In some embodiments, R² is imidazopyridinyl substituted with 1-2 independently selected R^{2A}. In some embodiments, R² is 1,3-dihydro-2H-benzo[d]imidazol-2-onyl substituted with 1-2 independently selected R^{2A}. In some embodiments, R² is [1,2,4]triazolo[1,5-a]pyridine substituted with 1-2 independently selected R^{2A}.

In some embodiments, one R^{2A} is C1-C6 alkyl, C1-C6 haloalkyl, C1-C6 hydroxyalkyl, cyano, or hydroxyl, and the other R^{2A} are independently selected from C1-C6 haloalkyl, C1-C6 hydroxyalkyl, cyano, hydroxyl, halogen, —NR^{A}R^{B}, —C(═O)NR^{A}R^{B},

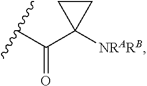

—NHC(═O)R^{C}, —C(═O)NR^{D}R^{E}, —C(═O)OR^{F}, —SO₂R^{F}, —NHSO₂R^{F}, —SO₂NR^{F}R^{G}, —NHC(═O)C1-C6 alkyl optionally substituted with NR^{A}R^{B}, 5-10 membered heteroaryl optionally substituted with 1-3 substituents independently selected from C1-C6 alkyl and —NR^{A}R^{B}, 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}, C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl, C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and C3-C6 cycloalkyl optionally substituted with 4-10 membered heterocyclyl optionally substituted with C1-C6 alkyl.

In some embodiments, each R^{2A} is independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1, 2, or 3 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1 or 2 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, R² is C1-C6 alkoxy optionally substituted with —C(═O)NR^{A}R^{C}. In some embodiments, R² is C1-C6 alkoxy substituted with —C(═O)NR^{A}R^{C}. In some embodiments, R² is C3-C6 alkoxy substituted with —C(═O)NR^{A}R^{C}. In some embodiments, R² is C1-C6 alkoxy.

In some embodiments, R² is C1-C6 alkylalkoxy optionally substituted with —C(═O)NR^{A}R^{C}. In some embodiments, R² is C1-C6 alkylalkoxy substituted with —C(═O)NR^{A}R^{C}.

In some embodiments, R² is C3-C6 alkylalkoxy substituted with —C(═O)NR^{A}R^{C}. In some embodiments, R² is C1-C6 alkylalkoxy.

In some embodiments, X is a bond.

In some embodiments, X is CH₂.

In some embodiments, X is CH(CH₃).

In some embodiments, X is C(CH₃)₂.

In some embodiments, X is

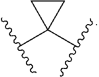 [SMILES: *C1(*)CC1]

In some embodiments, one of R^{3A} and R^{3B} is hydrogen and the other of R^{3A} and R^{3B} is C1-C6 alkyl. In some embodiments, one of R^{3A} and R^{3B} is hydrogen and the other of R^{3A} and R^{3B} is methyl. In some embodiments, each of R^{3A} and R^{3B} is hydrogen. In some embodiments, each of R^{3A} and R^{3B} is an independently selected C1-C6 alkyl. In some embodiments, each of R^{3A} and R^{3B} is methyl.

In some embodiments, one of R^{3A} and R^{3B} is hydrogen and the other of R^{3A} and R^{3B} is C1-C6 alkoxy. In some embodiments, one of R^{3A} and R^{3B} is C1-C6 alkyl and the other of R^{3A} and R^{3B} is C1-C6 alkoxy. In some embodiments, R^{3A} is C1-C6 alkoxy. In some embodiments, R^{3A} is C1-C3 alkoxy. In some embodiments, R^{3A} is —OCH₃, —OCH₂CH₃, or —OCH₂CH₂CH₃. In some embodiments, R^{3A} is —OCH₃.

In some embodiments, one of R^{3A} and R^{3B} is hydrogen and the other of R^{3A} and R^{3B} is C1-C6 haloalkyl. In some embodiments, one of R^{3A} and R^{3B} is C1-C6 alkyl and the other of R^{3A} and R^{3B} is C1-C6 haloalkyl. In some embodiments, R^{3A} is C1-C6 haloalkyl. In some embodiments, R^{3A} is C1-C3 haloalkyl. In some embodiments, R^{3A} is C1-C3 fluoroalkyl. In some embodiments, R^{3A} is CF₃. In some embodiments, R^{3A} is —CHF₂.

In some embodiments, R^{3A} and R^{3B}, together with the carbon and nitrogen atoms, respectively, to which they are attached together form a 4-8 membered heterocyclyl group. In some embodiments, R^{3A} and R^{3B}, together with the carbon and nitrogen atoms, respectively, to which they are attached together form a 5-6 membered heterocyclyl group.

In some embodiments, Y is phenyl optionally substituted with R^{Y}, naphthyl substituted with R^{Y}, or 5-10 membered heteroaryl substituted with R^{Y}.

In some embodiments, Y is phenyl optionally substituted with 1-3 independently selected R^{Y}. In some embodiments, Y is phenyl substituted with 1 or 2 independently selected R^{Y}.

In some embodiments, Y is phenyl substituted with 1 R^{Y}. In some embodiments, Y is phenyl substituted with 2 independently selected R^{Y}. In some embodiments, Y is phenyl optionally substituted with 3 independently selected R^{Y}. In some embodiments, Y is phenyl.

In some embodiments, Y is naphthyl optionally substituted with 1-3 independently selected R^{Y}. In some embodiments, Y is naphthyl substituted with 1 or 2 independently selected R^{Y}. In some embodiments, Y is naphthyl substituted with 1 R^{Y}. In some embodiments, Y is naphthyl substituted with 2 independently selected R^{Y}. In some embodiments, Y is naphthyl optionally substituted with 3 independently selected R^{Y}. In some embodiments, Y is naphthyl.

In some embodiments, Y is 5-10 membered heteroaryl optionally substituted with 1-3 independently selected R^{Y}. In some embodiments, Y is 5-10 membered heteroaryl substituted with 1 or 2 independently selected R^{Y}. In some embodiments, Y is 5-10 membered heteroaryl substituted with 1 R^{Y}. In some embodiments, Y is 5-10 membered heteroaryl substituted with 2 independently selected R^{Y}. In some embodiments, Y is 5-10 membered heteroaryl optionally substituted with 3 independently selected R^{Y}. In some embodiments, Y is 5-10 membered heteroaryl.

In some embodiments, Y is 6 membered heteroaryl optionally substituted with 1-3 independently selected R^{Y}. In some embodiments, Y is 6 membered heteroaryl substituted with 1 or 2 independently selected R^{Y}. In some embodiments, Y is 6 membered heteroaryl substituted with 1 R^{Y}. In some embodiments, Y is 6 membered heteroaryl substituted with 2 independently selected R^{Y}. In some embodiments, Y is 6 membered heteroaryl optionally substituted with 3 independently selected R^{Y}. In some embodiments, Y is 6 membered heteroaryl. In some embodiments, the 6 membered heteroaryl of Y is pyridyl (e.g., 3-pyridyl).

In some embodiments, Y is 9 membered heteroaryl optionally substituted with 1-3 independently selected R^{Y}. In some embodiments, Y is 9 membered heteroaryl substituted with 1 or 2 independently selected R^{Y}. In some embodiments, Y is 9 membered heteroaryl substituted with 1 R^{Y}. In some embodiments, Y is 9 membered heteroaryl substituted with 2 independently selected R^{Y}. In some embodiments, Y is 9 membered heteroaryl optionally substituted with 3 independently selected R^{Y}. In some embodiments, Y is 9 membered heteroaryl.

In some embodiments, 1, 2, or 3 of R is independently halogen. In some embodiments, 1, 2, or 3 of R^{Y} is independently chloro or fluoro. In some embodiments, 1 or 2 of R^{Y} is independently chloro or fluoro.

In some embodiments, 1, 2, or 3 of R is hydroxyl. In some embodiments, 1 or 2 of R is hydroxyl.

In some embodiments, 1, 2, or 3 of R^{Y} is cyano. In some embodiments, 1 or 2 of R^{Y} is cyano.

In some embodiments, 1, 2, or 3 of R^{Y} is independently C1-C6 haloalkyl. In some embodiments, 1 or 2 of R is independently C1-C3 haloalkyl. In some embodiments, 1 or 2 of R is trifluoromethyl.

In some embodiments, 1, 2, or 3 of R^{Y} is independently C1-C6 alkoxy. In some embodiments, 1 or 2 of R is independently C1-C3 alkoxy. In some embodiments, 1 or 2 of R is methoxy.

In some embodiments, 1, 2, or 3 of R^{Y} is independently C1-C6 haloalkoxy. In some embodiments, 1 or 2 of R is independently C1-C3 haloalkoxy. In some embodiments, 1 or 2 of R^{Y} is trifluoromethoxy.

In some embodiments, 1, 2, or 3 of R^{Y} is independently C1-C6 hydroxyalkyl. In some embodiments, 1 or 2 of R is independently C1-C3 hydroxyalkyl. In some embodiments, 1 or 2 of R^{Y} is independently mono-hydroxyl C1-C3 alkyl. In some embodiments, 1 or 2 of R^{Y} is independently di-hydroxyl C2-C3 alkyl.

In some embodiments, 1, 2, or 3 of R^{Y} is independently —NHC(═O)R^{C}. In some embodiments, 1 or 2 of R^{Y} is independently —NHC(═O)R^{C}. In some embodiments, 1 of R^{Y} is —NHC(═O)R^{C}. In some embodiments, Y is substituted with 1 R^{Y}, and R^{Y} is —NHC(═O)R^{C}.

In some embodiments, 1, 2, or 3 of R^{Y} is independently —C(═O)NHR. In some embodiments, 1 or 2 of R^{Y} is independently —C(═O)NHR^{Y1}. In some embodiments, 1 of R^{Y} is —C(═O)NHR^{Y1}. In some embodiments, Y is substituted with 1 R^{Y}, and R^{Y} is —C(═O)NHR^{Y1}.

In some embodiments, 1, 2, or 3 of R is independently —CO₂R^{A}. In some embodiments, 1 or 2 of R^{Y} is independently —CO₂R^{A}. In some embodiments, 1 of R^{Y} is —CO₂R^{A}. In some embodiments, Y is substituted with 1 R^{Y}, and R^{Y} is —CO₂R^{A}. In some embodiments, Y is substituted with 1 R^{Y}, and R^{Y} is —CO₂H.

In some embodiments, 1, 2, or 3 of R^{Y} is independently —SO₂NR^{F}R^{G}. In some embodiments, 1 or 2 of R^{Y} is independently —SO₂NR^{F}R^{G}. In some embodiments, 1 of R^{Y} is —SO₂NR^{F}R^{G}. In some embodiments, Y is substituted with 1 R^{Y}, and R^{Y} is —SO₂NR^{F}R^{G}.

In some embodiments, 1, 2, or 3 of R is independently —NHSO₂R^{F}. In some embodiments, 1 or 2 of R is independently —NHSO₂R^{F}. In some embodiments, 1 of R is —NHSO₂R^{F}. In some embodiments, Y is substituted with 1 R^{Y}, and R^{Y} is —NHSO₂R^{F}.

In some embodiments, 1, 2, or 3 of R^{Y} is independently —S(═O)(═NR^{F})R^{G}. In some embodiments, 1 or 2 of R is independently —S(═O)(═NR^{F})R^{G}. In some embodiments, 1 of R is —S(═O)(═NR^{F})R^{G}. In some embodiments, Y is substituted with 1 R, and R^{Y} is —S(═O)(═NR^{F})R^{G}.

In some embodiments, 1, 2, or 3 of R^{Y} is independently —SO₂(C1-C6 alkyl). In some embodiments, 1 or 2 of R is independently —SO₂(C1-C6 alkyl). In some embodiments, 1 of R is —SO₂(C1-C6 alkyl). In some embodiments, 1 or 2 of R is —SO₂CH₃. In some embodiments, 1 of R^{Y} is —SO₂CH₃. In some embodiments, Y is substituted with 1 R^{Y}, and R^{Y} is —SO₂CH₃.

In some embodiments, 1, 2, or 3 of R^{Y} is independently —C(═O)NR^{A}R^{B}. In some embodiments, 1 or 2 of R^{Y} is independently —C(═O)NR^{A}R^{B}. In some embodiments, 1 of R^{Y} is —C(═O)NR^{A}R^{B}. In some embodiments, Y is substituted with 1 R^{Y}, and R^{Y} is —C(═O)NR^{A}R^{B}.

In some embodiments, 1, 2, or 3 of R is independently 5-6 membered heteroaryl. In some embodiments, 1 of R is 5-6 membered heteroaryl. In some embodiments, Y is substituted with 1 R^{Y}, and R^{Y} is 5-6 membered heteroaryl.

In some embodiments, 1, 2, or 3 of R^{Y} is independently heteroaralkyl. In some embodiments, 1 of R is independently heteroaralkyl.

In some embodiments, 1, 2, or 3 of R is independently C1-C6 alkyl optionally substituted with —CO₂R^{A} or 5-6 membered heteroaryl optionally substituted with R^{Y1}. In some embodiments, 1, 2, or 3 of R is independently C1-C6 alkyl substituted with —CO₂R^{A} or 5-6 membered heteroaryl optionally substituted with R^{Y1}. In some embodiments, 1, 2, or 3 of R is independently C1-C6 alkyl substituted with —CO₂R^{A} or 5-6 membered heteroaryl substituted with R^{Y1}. In some embodiments, 1, 2, or 3 of R^{Y} is independently C1-C6 alkyl substituted with —CO₂R^{A} or 5-6 membered heteroaryl. In some embodiments, Y is substituted with 1 R^{Y}, and R^{Y} is C1-C6 alkyl substituted with —CO₂R^{A}. In some embodiments, Y is substituted with 1 R^{Y}, and R^{Y} is C1-C6 alkyl substituted with —CO₂H.

In some embodiments, 1 or 2 of R^{Y} is independently C1-C6 alkyl optionally substituted with —CO₂R^{A} or 5-6 membered heteroaryl optionally substituted with R^{Y1}. In some embodiments, 1 or 2 of R is independently C1-C6 alkyl substituted with —CO₂R^{A} or 5-6 membered heteroaryl optionally substituted with R^{Y1}. In some embodiments, 1 or 2 of R is independently C1-C6 alkyl substituted with —CO₂R^{A} or 5-6 membered heteroaryl substituted with R^{Y1}. In some embodiments, 1 or 2 of R is independently C1-C6 alkyl substituted with —CO₂R^{A} or 5-6 membered heteroaryl.

In some embodiments, 1 or 2 of R is independently C1-C6 alkyl substituted with —CO₂R^{A}. In some embodiments, 1 or 2 of R^{Y} is independently C1-C6 alkyl substituted 5-6 membered heteroaryl optionally substituted with R^{Y1}. In some embodiments, 1 or 2 of R is independently C1-C6 alkyl substituted with 5-6 membered heteroaryl substituted with R^{Y1}. In some embodiments, 1 or 2 of R^{Y} is independently C1-C6 alkyl substituted with 5-6 membered heteroaryl.

In some embodiments, 1, 2, or 3 of R^{Y} is independently C1-C6 alkyl. In some embodiments, 1 or 2 of R is independently C1-C3 alkyl. In some embodiments, 1, 2, or 3 of R is methyl.

In some embodiments, R^{Y1} is —SO₂(C1-C6 alkyl). In some embodiments, R^{Y1} is —SO₂CH₃.

In some embodiments, R^{Y1} is hydroxyl.

In some embodiments, R^{Y1} is C1-C6 alkyl optionally substituted with oxo. In some embodiments, R^{Y1} is C1-C6 alkyl substituted with oxo. In some embodiments, R^{Y1} is acetyl, 1-oxoethyl, or 1-oxopropyl. In some embodiments, R^{Y1} is C1-C6 alkyl. In some embodiments, R^{Y1} is methyl.

In some embodiments, R⁴ is hydrogen.

In some embodiments, R⁴ is C1-C6 alkyl. In some embodiments, R⁴ is methyl or ethyl. In some embodiments, R⁴ is methyl.

In some embodiments, R⁴ is acrylamido.

In some embodiments, R¹ is hydrogen.

In some embodiments, R⁵ is C1-C6 alkyl. In some embodiments, R⁵ is methyl or ethyl. In some embodiments, R⁵ is methyl.

In some embodiments, R⁵ is halogen. In some embodiments, R⁵ is fluoro or chloro. In some embodiments, R⁵ is fluoro. In some embodiments, R⁵ is chloro.

In some embodiments, R⁵ is C1-C6 haloalkyl. In some embodiments, R⁵ is trifluoromethyl.

In some embodiments, R⁵ is C3-C6 cycloalkyl. In some embodiments, R⁵ is cyclopropyl.

In some embodiments, R⁵ is cyano.

In some embodiments, R⁵ is —NR^{5A}R^{5B}.

In some embodiments, R⁵ is —NR^{5A}C(═O)R^{5B}.

In some embodiments, R⁵ is —C(═O)NR^{5A}R^{5B}.

In some embodiments, one of R^{5A} and R^{5B} is hydrogen and the other of R^{5A} and R^{5B} is C1-C6 alkyl, C2-C6 alkenyl, or C1-C6 hydroxyalkyl. In some embodiments, one of R^{5A} and R^{5B} is C1-C6 alkyl and the other of R^{5A} and R^{5B} is C1-C6 alkyl, C2-C6 alkenyl, or C1-C6 hydroxyalkyl. In some embodiments, each of R^{5A} and R^{5B} is hydrogen. In some embodiments, each of R^{5A} and R^{5B} is an independently selected C1-C6 alkyl. In some embodiments, each of R^{5A} and R^{5B} is methyl. In some embodiments, the C1-C6 hydroxyalkyl of R^{5A} and R^{5B} is hydroxymethyl, 1-hydroxyethyl, 2-hydroxyethyl, dihydroxypropyl or dihydroxybutyl.

In some embodiments, R⁵ is acrylamido.

In some embodiments, R⁶ is hydrogen.

In some embodiments, R⁶ is halogen. In some embodiments, R⁶ is fluoro. In some embodiments, R⁶ is chloro.

In some embodiments, R⁶ is C1-C6 alkyl. In some embodiments, R⁶ is methyl.

In some embodiments, each of R^{A} and R^{B} are independently selected from hydrogen, hydroxyl, C1-C6 alkoxy, C3-C6 cycloalkyl, C2-C6 alkenyl, cyano, 4-10 membered heterocyclyl, and C1-C6 alkyl optionally substituted with hydroxyl or C1-C6 alkoxy.

In some embodiments, R^{A} and R^{B} together with the nitrogen atom to which they are attached form a 4-10 membered heterocyclyl optionally substituted with 1-2 substituents independently selected from halogen, C1-C6 alkyl, hydroxyl, and —C(═O)C1-C6 alkyl.

In some embodiments, each of R^{A} and R^{B} are independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

In some embodiments, one of R^{A} and R^{B} is hydrogen and the other of R^{A} and R^{B} is hydroxyl. In some embodiments, one of R^{A} and R^{B} is hydrogen and the other of R^{A} and R^{B} is C1-C6 alkoxy. In some embodiments, one of R^{A} and R^{B} is hydrogen and the other of R^{A} and R^{B} is C3-C6 cycloalkyl. In some embodiments, one of R^{A} and R^{B} is hydrogen and the other of R^{A} and R^{B} is C2-C6 alkenyl. In some embodiments, one of R^{A} and R^{B} is hydrogen and the other of R^{A} and R^{B} is C1-C6 alkyl optionally substituted with hydroxyl or C1-C6 alkoxy. In some embodiments, one of R^{A} and R^{B} is hydrogen and the other of R^{A} and R^{B} is C1-C6 alkyl substituted with hydroxyl. In some embodiments, one of R^{A} and R^{B} is hydrogen and the other of R^{A} and R^{B} is C1-C6 alkyl substituted with C1-C6 alkoxy. In some embodiments, one of R^{A} and R^{B} is hydrogen and the other of R^{A} and R^{B} is 4-10 membered heterocyclyl.

In some embodiments, one of R^{A} and R^{B} is hydrogen and the other of R^{A} and R^{B} is C1-C6 alkyl. In some embodiments, each of R^{A} and R^{B} are hydrogen. In some embodiments, each of R^{A} and R^{B} are an independently selected C1-C6 alkyl optionally substituted with hydroxyl or C1-C6 alkoxy. In some embodiments, each of R^{A} and R^{B} are an independently selected C1-C6 alkyl. In some embodiments, each of R^{A} and R^{B} are methyl.

In some embodiments, R^{A} and R^{B} together with the nitrogen atom to which they are attached form a 4-10 membered heterocyclyl optionally substituted with 1-2 substituents independently selected from halogen, C1-C6 alkyl, hydroxyl, and —C(═O)C1-C6 alkyl.

In some embodiments, R^{A} and R^{B} together with the nitrogen atom to which they are attached form a 4-10 membered heterocyclyl substituted with 1-2 substituents independently selected from halogen, C1-C6 alkyl, hydroxyl, and —C(═O)C1-C6 alkyl.

In some embodiments, R^{A} and R^{B} together with the nitrogen atom to which they are attached form a 4-10 membered heterocyclyl substituted with halogen, C1-C6 alkyl, hydroxyl, or —C(═O)C1-C6 alkyl. In some embodiments, R^{A} and R^{B} together with the nitrogen atom to which they are attached form a 4-10 membered heterocyclyl substituted with 1-2 substitutents independently selected from fluoro, hydroxyl, methyl, and acetyl.

In some embodiments, R^{A} and R^{B} together with the nitrogen atom to which they are attached form a 4-10 membered heterocyclyl. In some embodiments, R^{A} and R^{B} together with the nitrogen atom to which they are attached form a 4-6 membered heterocyclyl.

In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl optionally substituted with C1-C6 alkyl or with C1-C6 hydroxylalkyl. In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl substituted with C1-C6 alkyl or with C1-C6 hydroxylalkyl. In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl. In some embodiments, each R^{C} is independently C3-C6 cycloalkyl, —C(═O)NHR^{Y1}, or a C1-C6 alkyl.

In some embodiments, each R^{C} is independently C3-C6 cycloalkyl. In some embodiments, each R^{C} is independently —C(═O)NHR^{Y1}. In some embodiments, each R^{C} is independently C1-C6 alkyl optionally substituted with —NR^{A}R^{B} or with 4-10 membered heterocyclyl optionally substituted with C1-C6 alkyl or with C1-C6 hydroxylalkyl. In some embodiments, each R^{C} is independently C1-C6 alkyl substituted with —NR^{A}R^{B} or with 4-10 membered heterocyclyl optionally substituted with C1-C6 alkyl or with C1-C6 hydroxylalkyl.

In some embodiments, R^{D} is selected from hydrogen, hydroxyl, C1-C6 alkyl, phenyl, 4-10 membered heterocyclyl, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy.

In some embodiments, R^{D} is hydrogen.

In some embodiments, R^{D} is hydroxyl.

In some embodiments, R^{D} is C1-C6 alkyl optionally substituted with 4-10 membered heterocyclyl, C3-C6 cycloalkyl, or cyano. In some embodiments, R^{D} is unsubstituted C1-C6 alkyl.

In some embodiments, R^{D} is C1-C6 alkyl substituted with 4-10 membered heterocyclyl, C3-C6 cycloalkyl, or cyano. In some embodiments, R^{D} is C1-C6 alkyl substituted with 4-10 membered heterocyclyl. In some embodiments, R^{D} is C1-C6 alkyl substituted with C3-C6 cycloalkyl. In some embodiments, R^{D} is C1-C6 alkyl substituted with cyano.

In some embodiments, R^{D} is 4-10 membered heterocyclyl optionally substituted with 4-10 membered heterocyclyl, C3-C6 cycloalkyl, or cyano. In some embodiments, R^{D} is unsubstituted 4-10 membered heterocyclyl. In some embodiments, R^{D} is 4-10 membered heterocyclyl substituted with 4-10 membered heterocyclyl, C3-C6 cycloalkyl, or cyano. In some embodiments, R^{D} is 4-10 membered heterocyclyl substituted with 4-10 membered heterocyclyl. In some embodiments, R^{D} is 4-10 membered heterocyclyl substituted with C3-C6 cycloalkyl. In some embodiments, R^{D} is 4-10 membered heterocyclyl substituted with cyano.

In some embodiments, R^{D} is phenyl optionally substituted with 4-10 membered heterocyclyl, C3-C6 cycloalkyl, or cyano. In some embodiments, R^{D} is unsubstituted phenyl. In some embodiments, R^{D} is phenyl substituted with 4-10 membered heterocyclyl, C3-C6 cycloalkyl, or cyano. In some embodiments, R^{D} is phenyl substituted with 4-10 membered heterocyclyl. In some embodiments, R^{D} is phenyl substituted with C3-C6 cycloalkyl. In some embodiments, R^{D} is phenyl substituted with cyano.

In some embodiments, R^{D} is C3-C6 cycloalkyl optionally substituted with C1-C6 alkyl optionally substituted with hydroxyl or C1-C6 alkoxy. In some embodiments, R^{D} is unsubstituted C3-C6 cycloalkyl. In some embodiments, R^{D} is C3-C6 cycloalkyl substituted with C1-C6 alkyl optionally substituted with hydroxyl or C1-C6 alkoxy. In some embodiments, R^{D} is C3-C6 cycloalkyl substituted with C1-C6 alkyl. In some embodiments, R^{D} is C3-C6 cycloalkyl substituted with C1-C6 alkyl substituted with hydroxyl. In some embodiments, R^{D} is C3-C6 cycloalkyl substituted with C1-C6 alkyl substituted with C1-C6 alkoxy.

In some embodiments, R^{D} is C1-C6 alkoxy.

In some embodiments, R^{D} is C3-C6 cycloalkyl.

In some embodiments, R^{E} is selected from hydrogen, hydroxyl, C1-C6 alkyl, phenyl, 4-10 membered heterocyclyl, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy.

In some embodiments, R^{E} is hydrogen.

In some embodiments, R^{E} is hydroxyl.

In some embodiments, R^{E} is C1-C6 alkyl optionally substituted with 4-10 membered heterocyclyl, C3-C6 cycloalkyl, or cyano. In some embodiments, R^{E} is unsubstituted C1-C6 alkyl. In some embodiments, R^{E} is C1-C6 alkyl substituted with 4-10 membered heterocyclyl, C3-C6 cycloalkyl, or cyano. In some embodiments, R^{E} is C1-C6 alkyl substituted with 4-10 membered heterocyclyl. In some embodiments, R^{E} is C1-C6 alkyl substituted with C3-C6 cycloalkyl. In some embodiments, R^{E} is C1-C6 alkyl substituted with cyano.

In some embodiments, R^{E} is 4-10 membered heterocyclyl optionally substituted with 4-10 membered heterocyclyl, C3-C6 cycloalkyl, or cyano. In some embodiments, R^{E} is unsubstituted 4-10 membered heterocyclyl. In some embodiments, R^{E} is 4-10 membered heterocyclyl substituted with 4-10 membered heterocyclyl, C3-C6 cycloalkyl, or cyano. In some embodiments, R^{E} is 4-10 membered heterocyclyl substituted with 4-10 membered heterocyclyl. In some embodiments, R^{E} is 4-10 membered heterocyclyl substituted with C3-C6 cycloalkyl. In some embodiments, R^{E} is 4-10 membered heterocyclyl substituted with cyano.

In some embodiments, R^{E} is phenyl optionally substituted with 4-10 membered heterocyclyl, C3-C6 cycloalkyl, or cyano. In some embodiments, R^{E} is unsubstituted phenyl. In some embodiments, R^{E} is phenyl substituted with 4-10 membered heterocyclyl, C3-C6 cycloalkyl, or cyano. In some embodiments, R^{E} is phenyl substituted with 4-10 membered heterocyclyl. In some embodiments, R^{E} is phenyl substituted with C3-C6 cycloalkyl. In some embodiments, R^{E} is phenyl substituted with cyano.

In some embodiments, R^{E} is C3-C6 cycloalkyl optionally substituted with C1-C6 alkyl optionally substituted with hydroxyl or C1-C6 alkoxy. In some embodiments, R^{E} is unsubstituted C3-C6 cycloalkyl. In some embodiments, R^{E} is C3-C6 cycloalkyl substituted with C1-C6 alkyl optionally substituted with hydroxyl or C1-C6 alkoxy. In some embodiments, R^{E} is C3-C6 cycloalkyl substituted with C1-C6 alkyl. In some embodiments, R^{E} is C3-C6 cycloalkyl substituted with C1-C6 alkyl substituted with hydroxyl. In some embodiments, R^{E} is C3-C6 cycloalkyl substituted with C1-C6 alkyl substituted with C1-C6 alkoxy.

In some embodiments, R^{E} is C1-C6 alkoxy.

In some embodiments, R^{E} is C3-C6 cycloalkyl.

In some embodiments, one of R^{D} and R^{E} is hydrogen and the other of R^{D} and R^{E} is hydrogen, hydroxyl, C1-C6 alkyl, phenyl, 4-10 membered heterocyclyl, C1-C6 alkoxy, and C3—C6 cycloalkyl optionally substituted with C1-C6 alkyl optionally substituted with hydroxyl or C1-C6 alkoxy.

In some embodiments, one of R^{D} and R^{E} is hydrogen and the other of R^{D} and R^{E} is hydroxyl, C1-C6 alkyl, or C1-C6 alkoxy. In some embodiments, one of R^{D} and R^{E} is hydrogen and the other of R^{D} and R^{E} is hydroxyl. In some embodiments, one of R^{D} and R^{E} is hydrogen and the other of R^{D} and R^{E} is C1-C6 alkyl. In some embodiments, one of R^{D} and R^{E} is hydrogen and the other of R^{D} and R^{E} is C1-C6 alkoxy. In some embodiments, one of R^{D} and R^{E} is hydrogen and the other of R^{D} and R^{E} is hydroxyl, methyl, or methoxy. In some embodiments, each of R^{D} and R^{E} is hydrogen. In some embodiments, each of R^{D} and R^{E} is an independently selected C1-C6 alkyl. In some embodiments, each of R^{D} and R^{E} is methyl.

In some embodiments, one of R^{E} and R^{G} is hydrogen and the other of R^{F} and R^{G} is phenyl or C1-C6 alkyl optionally substituted with oxo or —NR^{A}R^{B}. In some embodiments, one of R^{F} and R^{G} is hydrogen and the other of R^{E} and R^{G} is phenyl or C1-C6 alkyl substituted with oxo or —NR^{A}R^{B}. In some embodiments, one of R^{F} and R^{G} is hydrogen and the other of R^{F} and R^{G} is phenyl or C1-C6 alkyl. In some embodiments, one of R^{F} and R^{G} is hydrogen and the other of R^{F} and R^{G} is phenyl. In some embodiments, one of R^{F} and R^{G} is hydrogen and the other of R^{F} and R^{G} is C1-C6 alkyl optionally substituted with oxo or —NR^{A}R^{B}. In some embodiments, one of R^{F} and R^{G} is hydrogen and the other of R^{F} and R^{G} is C1-C6 alkyl substituted with oxo or —NR^{A}R^{B}.

In some embodiments, each of R^{F} and R^{G} is hydrogen. In some embodiments, each of R^{E} and R^{G} is an independently selected C1-C6 alkyl. In some embodiments, each of R^{F} and R^{G} is methyl.

In some embodiments, the compound of Formula (I), or a pharmaceutically acceptable salt thereof, is a compound of Formula (I-A1A):

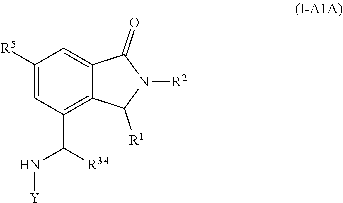

or a pharmaceutically acceptable salt thereof.

In some embodiments, the compound of Formula (I), or a pharmaceutically acceptable salt thereof, is a compound of Formula (I-A2):

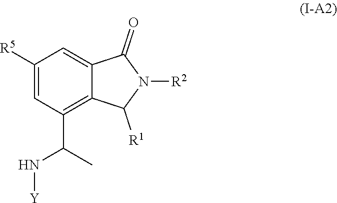

or a pharmaceutically acceptable salt thereof.

In some embodiments, the compound of Formula (I), or a pharmaceutically acceptable salt thereof, is a compound of Formula (I-A3):

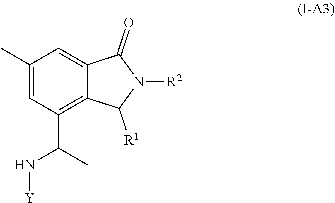

or a pharmaceutically acceptable salt thereof.

In some embodiments, the compound of Formula (I), or a pharmaceutically acceptable salt thereof, is a compound of Formula (I-A4):

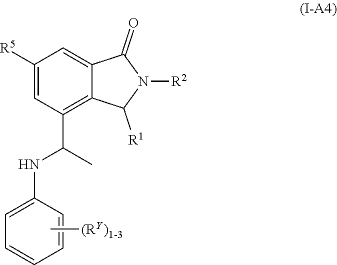

or a pharmaceutically acceptable salt thereof.

In some embodiments, the compound of Formula (I), or a pharmaceutically acceptable salt thereof, is a compound of Formula (I-A5):

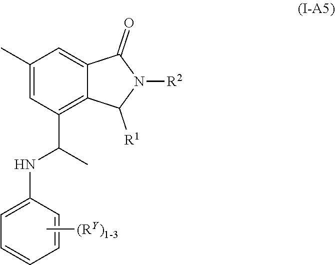

or a pharmaceutically acceptable salt thereof.

In some embodiments, the compound of Formula (I), or a pharmaceutically acceptable salt thereof, is a compound of Formula (I-A6):

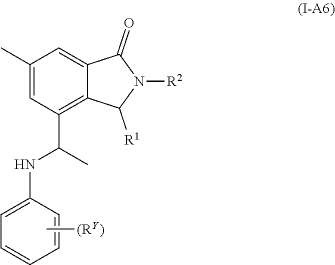

or a pharmaceutically acceptable salt thereof.

In some embodiments, the compound of Formula (I), or a pharmaceutically acceptable salt thereof, is a compound of Formula (I-A7):

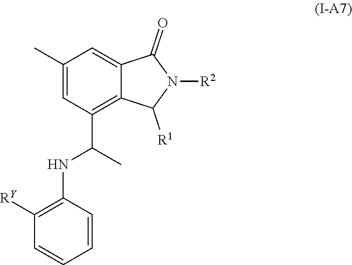

or a pharmaceutically acceptable salt thereof.

In some embodiments, the compound of Formula (I), or a pharmaceutically acceptable salt thereof, is a compound of Formula (I-A8):

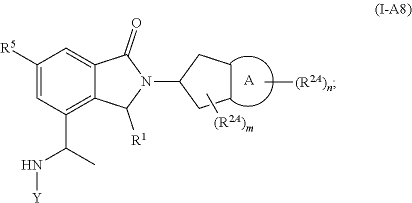

or a pharmaceutically acceptable salt thereof, wherein:
Ring A is a 5-6 membered heterocyclyl, C5-C6 cycloalkyl, or 5-6 membered heteroaryl; and
m is 0, 1, 2, or 3; n is 0, 1, or 2; and m+n is 0, 1, 2, or 3.

In some embodiments, Ring A is pyridyl, tetrahydropyranyl, or cyclohexyl. In some embodiments, Ring A is pyridyl. In some embodiments, Ring A is tetrahydropyranyl. In some embodiments, Ring A is cyclohexyl.

In some embodiments, the compound of Formula (I), or a pharmaceutically acceptable salt thereof, is a compound of Formula (I-A9):

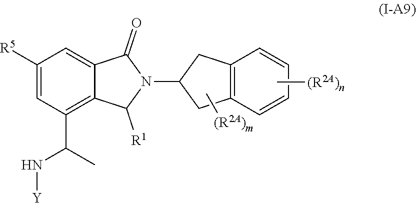

or a pharmaceutically acceptable salt thereof, wherein:
m is 0, 1, 2, or 3; n is 0, 1, or 2; and m+n is 0, 1, 2, or 3.

In some embodiments, the compound of Formula (I), or a pharmaceutically acceptable salt thereof, is a compound of Formula (I-A10):

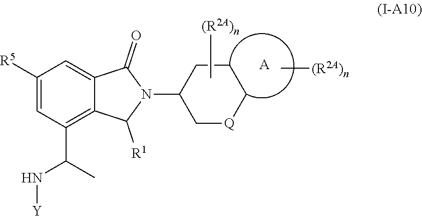

or a pharmaceutically acceptable salt thereof, wherein:
Q is CH, NH, or O;
Ring A is phenyl or 5-6 membered heteroaryl; and
m is 0, 1, 2, or 3; n is 0, 1, or 2; and m+n is 0, 1, 2, or 3.

In some embodiments, Q is CH. In some embodiments, Q is NH. In some embodiments, Q is O.

In some embodiments, Ring A is phenyl.

In some embodiments, Ring A is pyridyl or pyrimidinyl.

### Non-Limiting Exemplary Compounds

In some embodiments, the compound is selected from the group consisting of the compounds in Example 1 (e.g., Compound 1), or a pharmaceutically acceptable salt thereof.

In some embodiments, the compound is selected from the group consisting of the compounds delineated in Table A, or a pharmaceutically acceptable salt thereof.

TABLE A

| Compound | |
|---|---|
| Number | Structure |
| 1 | 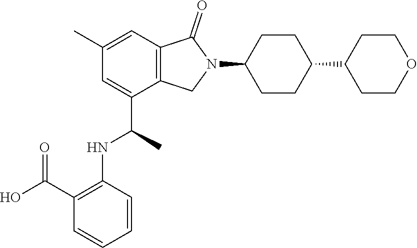 [SMILES: Cc1cc2c(c([C@@H](C)Nc3ccccc3C(=O)O)c1)CN([C@H]1CC[C@H](C3CCOCC3)CC1)C2=O] |
| 2 | 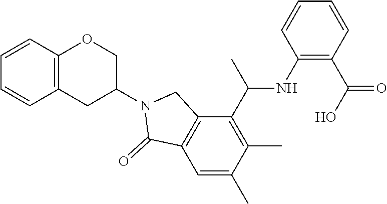 [SMILES: Cc1cc2c(c(C(C)Nc3ccccc3C(=O)O)c1C)CN(C1COc3ccccc3C1)C2=O] |
| 3 | 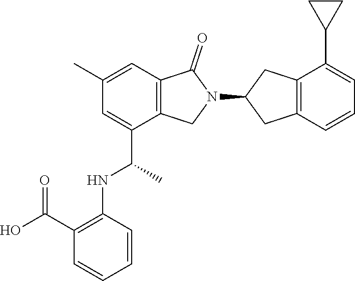 [SMILES: Cc1cc2c(c([C@H](C)Nc3ccccc3C(=O)O)c1)CN([C@@H]1Cc3cccc(C4CC4)c3C1)C2=O] |
| 4 | 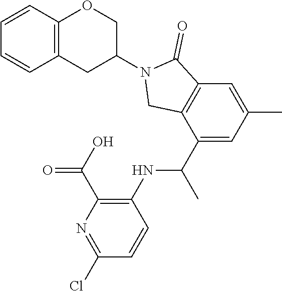 [SMILES: Cc1cc2c(c(C(C)Nc3ccc(Cl)nc3C(=O)O)c1)CN(C1COc3ccccc3C1)C2=O] |
| 5 | 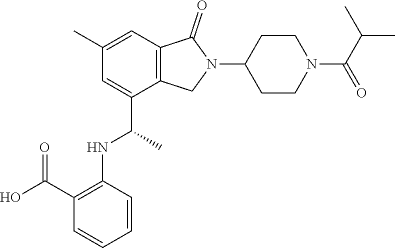 [SMILES: Cc1cc2c(c([C@H](C)Nc3ccccc3C(=O)O)c1)CN(C1CCN(C(=O)C(C)C)CC1)C2=O] |
| 6 | 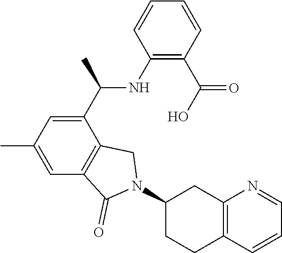 [SMILES: Cc1cc2c(c([C@@H](C)Nc3ccccc3C(=O)O)c1)CN([C@@H]1CCc3cccnc3C1)C2=O] |
| 7 | 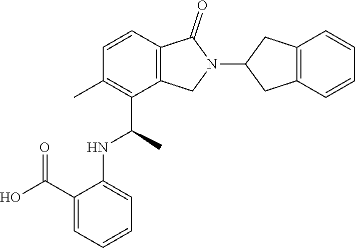 [SMILES: Cc1ccc2c(c1[C@@H](C)Nc1ccccc1C(=O)O)CN(C1Cc3ccccc3C1)C2=O] |
| 8 | 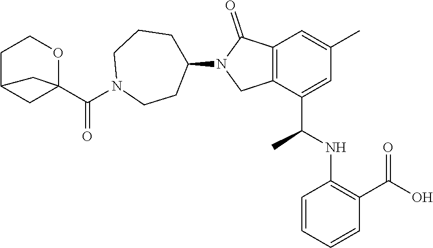 [SMILES: Cc1cc2c(c([C@H](C)Nc3ccccc3C(=O)O)c1)CN([C@@H]1CCCN(C(=O)C34CC(CCO3)C4)CC1)C2=O] |
| 9 | 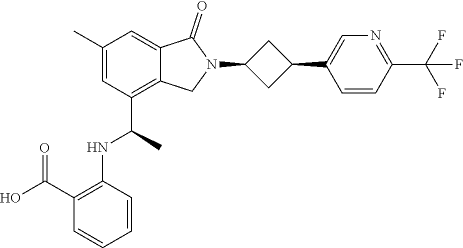 [SMILES: Cc1cc2c(c([C@@H](C)Nc3ccccc3C(=O)O)c1)CN([C@H]1C[C@@H](c3ccc(C(F)(F)F)nc3)C1)C2=O] |
| 10 | 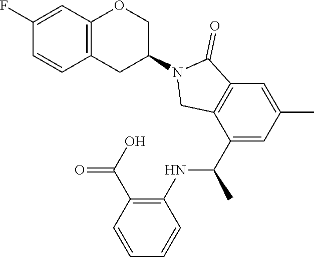 [SMILES: Cc1cc2c(c([C@@H](C)Nc3ccccc3C(=O)O)c1)CN([C@@H]1COc3cc(F)ccc3C1)C2=O] |
| 11 | 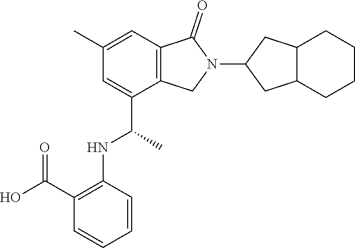 [SMILES: Cc1cc2c(c([C@H](C)Nc3ccccc3C(=O)O)c1)CN(C1CC3CCCCC3C1)C2=O] |
| 12 | 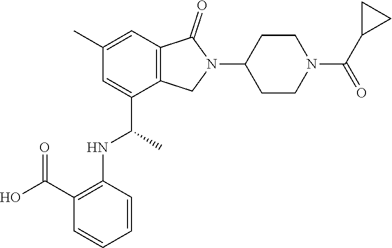 [SMILES: Cc1cc2c(c([C@H](C)Nc3ccccc3C(=O)O)c1)CN(C1CCN(C(=O)C3CC3)CC1)C2=O] |
| 13 | 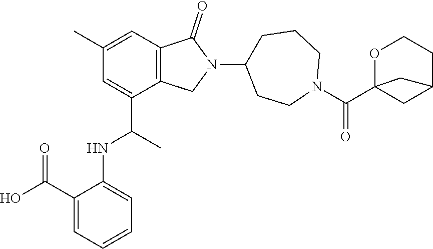 [SMILES: Cc1cc2c(c(C(C)Nc3ccccc3C(=O)O)c1)CN(C1CCCN(C(=O)C34CC(CCO3)C4)CC1)C2=O] |
| 14 | 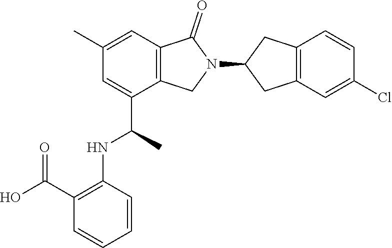 [SMILES: Cc1cc2c(c([C@@H](C)Nc3ccccc3C(=O)O)c1)CN([C@H]1Cc3ccc(Cl)cc3C1)C2=O] |
| 15 | 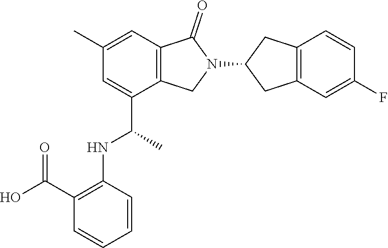 [SMILES: Cc1cc2c(c([C@H](C)Nc3ccccc3C(=O)O)c1)CN([C@@H]1Cc3ccc(F)cc3C1)C2=O] |
| 16 | 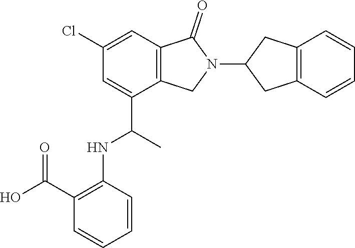 [SMILES: CC(Nc1ccccc1C(=O)O)c1cc(Cl)cc2c1CN(C1Cc3ccccc3C1)C2=O] |
| 17 | 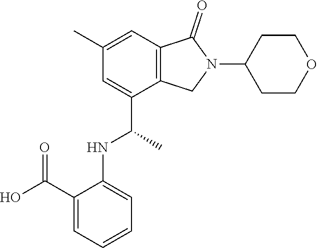 [SMILES: Cc1cc2c(c([C@H](C)Nc3ccccc3C(=O)O)c1)CN(C1CCOCC1)C2=O] |
| 18 | 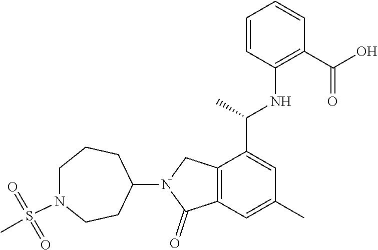 [SMILES: Cc1cc2c(c([C@H](C)Nc3ccccc3C(=O)O)c1)CN(C1CCCN(S(C)(=O)=O)CC1)C2=O] |
| 19 | 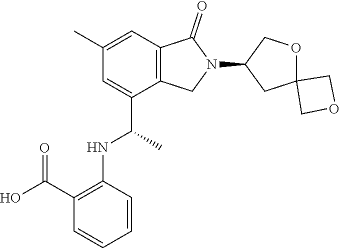 [SMILES: Cc1cc2c(c([C@H](C)Nc3ccccc3C(=O)O)c1)CN([C@H]1COC3(COC3)C1)C2=O] |
| 20 | 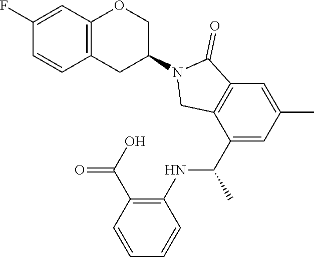 [SMILES: Cc1cc2c(c([C@H](C)Nc3ccccc3C(=O)O)c1)CN([C@@H]1COc3cc(F)ccc3C1)C2=O] |
| 21 | 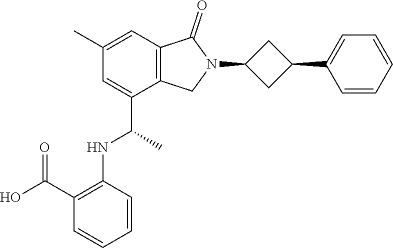 [SMILES: Cc1cc2c(c([C@H](C)Nc3ccccc3C(=O)O)c1)CN([C@H]1C[C@@H](c3ccccc3)C1)C2=O] |
| 22 | 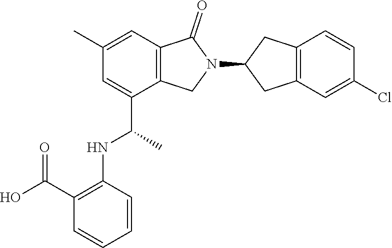 [SMILES: Cc1cc2c(c([C@H](C)Nc3ccccc3C(=O)O)c1)CN([C@H]1Cc3ccc(Cl)cc3C1)C2=O] |
| 23 | 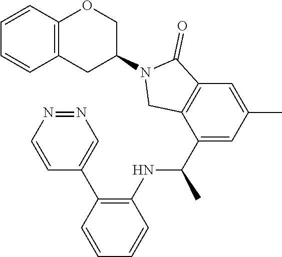 [SMILES: Cc1cc2c(c([C@@H](C)Nc3ccccc3-c3ccnnc3)c1)CN([C@@H]1COc3ccccc3C1)C2=O] |
| 24 | 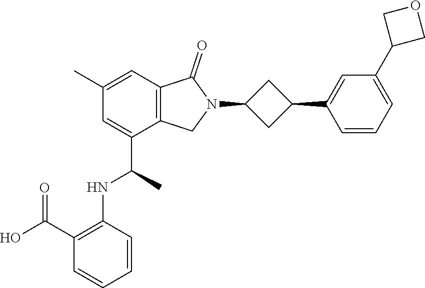 [SMILES: Cc1cc2c(c([C@@H](C)Nc3ccccc3C(=O)O)c1)CN([C@H]1C[C@@H](c3cccc(C4COC4)c3)C1)C2=O] |
| 25 | 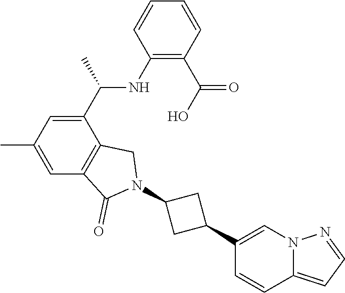 [SMILES: Cc1cc2c(c([C@H](C)Nc3ccccc3C(=O)O)c1)CN([C@H]1C[C@@H](c3ccc4ccnn4c3)C1)C2=O] |
| 26 | 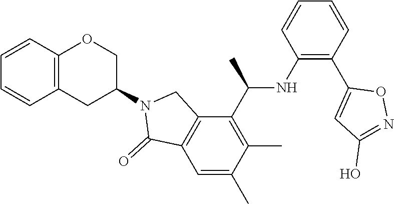 [SMILES: Cc1cc2c(c([C@@H](C)Nc3ccccc3-c3cc(O)no3)c1C)CN([C@@H]1COc3ccccc3C1)C2=O] |
| 27 | 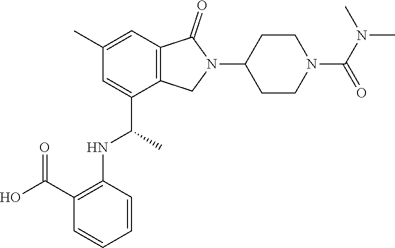 [SMILES: Cc1cc2c(c([C@H](C)Nc3ccccc3C(=O)O)c1)CN(C1CCN(C(=O)N(C)C)CC1)C2=O] |
| 28 | 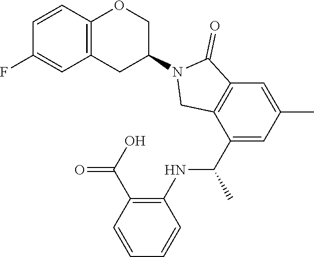 [SMILES: Cc1cc2c(c([C@H](C)Nc3ccccc3C(=O)O)c1)CN([C@@H]1COc3ccc(F)cc3C1)C2=O] |
| 29 | 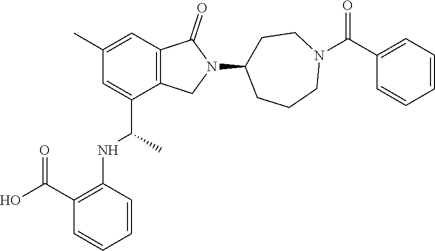 [SMILES: Cc1cc2c(c([C@H](C)Nc3ccccc3C(=O)O)c1)CN([C@@H]1CCCN(C(=O)c3ccccc3)CC1)C2=O] |
| 30 | 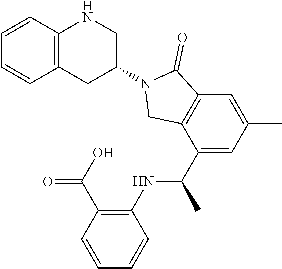 [SMILES: Cc1cc2c(c([C@@H](C)Nc3ccccc3C(=O)O)c1)CN([C@H]1CNc3ccccc3C1)C2=O] |
| 31 | 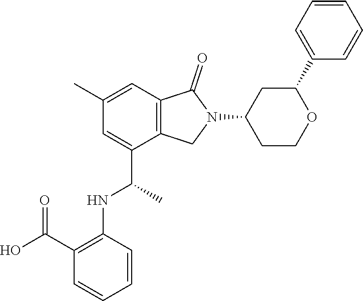 [SMILES: Cc1cc2c(c([C@H](C)Nc3ccccc3C(=O)O)c1)CN([C@H]1CCO[C@@H](c3ccccc3)C1)C2=O] |
| 32 | 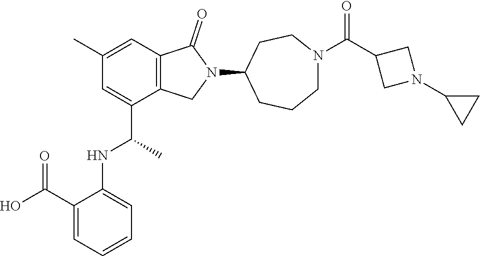 [SMILES: Cc1cc2c(c([C@H](C)Nc3ccccc3C(=O)O)c1)CN([C@@H]1CCCN(C(=O)C3CN(C4CC4)C3)CC1)C2=O] |
| 33 | 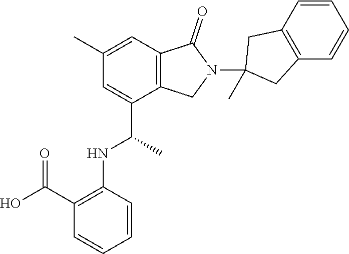 [SMILES: Cc1cc2c(c([C@H](C)Nc3ccccc3C(=O)O)c1)CN(C1(C)Cc3ccccc3C1)C2=O] |
| 34 | 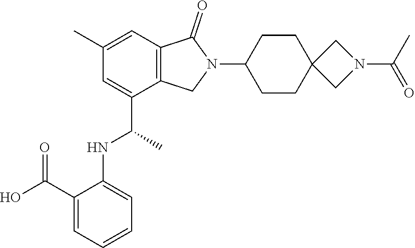 [SMILES: CC(=O)N1CC2(CCC(N3Cc4c(cc(C)cc4[C@H](C)Nc4ccccc4C(=O)O)C3=O)CC2)C1] |
| 35 | 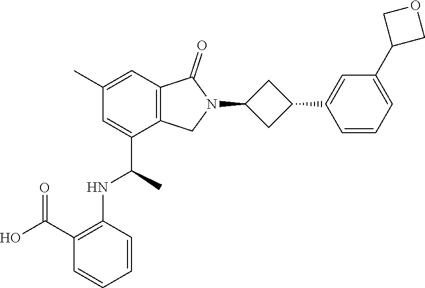 [SMILES: Cc1cc2c(c([C@@H](C)Nc3ccccc3C(=O)O)c1)CN([C@H]1C[C@H](c3cccc(C4COC4)c3)C1)C2=O] |
| 36 | 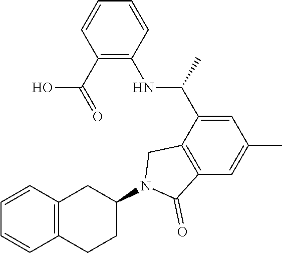 [SMILES: Cc1cc2c(c([C@@H](C)Nc3ccccc3C(=O)O)c1)CN([C@H]1CCc3ccccc3C1)C2=O] |
| 37 | 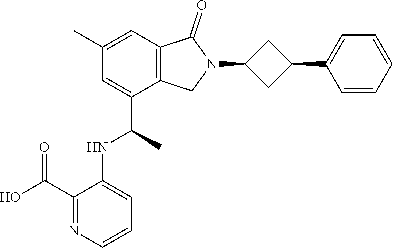 [SMILES: Cc1cc2c(c([C@@H](C)Nc3cccnc3C(=O)O)c1)CN([C@H]1C[C@@H](c3ccccc3)C1)C2=O] |
| 38 | 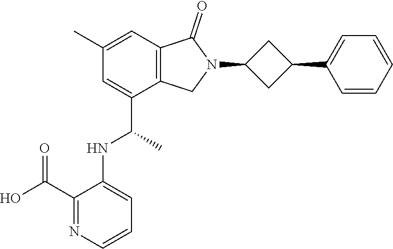 [SMILES: Cc1cc2c(c([C@H](C)Nc3cccnc3C(=O)O)c1)CN([C@H]1C[C@@H](c3ccccc3)C1)C2=O] |
| 39 | 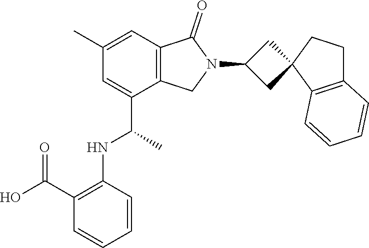 [SMILES: Cc1cc2c(c([C@H](C)Nc3ccccc3C(=O)O)c1)CN([C@H]1C[C@@]3(CCc4ccccc43)C1)C2=O] |
| 40 | 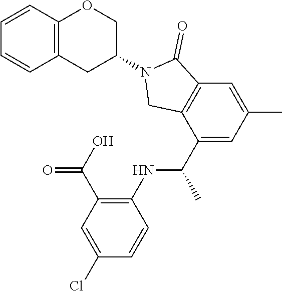 [SMILES: Cc1cc2c(c([C@H](C)Nc3ccc(Cl)cc3C(=O)O)c1)CN([C@H]1COc3ccccc3C1)C2=O] |
| 41 | 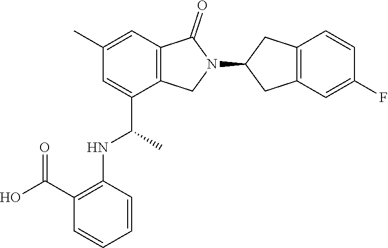 [SMILES: Cc1cc2c(c([C@H](C)Nc3ccccc3C(=O)O)c1)CN([C@H]1Cc3ccc(F)cc3C1)C2=O] |
| 42 | 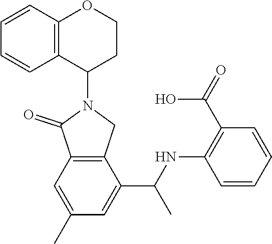 [SMILES: Cc1cc2c(c(C(C)Nc3ccccc3C(=O)O)c1)CN(C1CCOc3ccccc31)C2=O] |
| 43 | 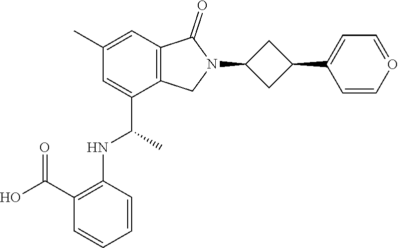 |
| 44 | 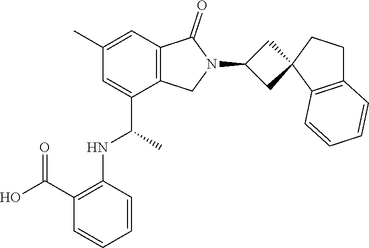 [SMILES: Cc1cc2c(c([C@H](C)Nc3ccccc3C(=O)O)c1)CN([C@H]1C[C@@]3(CCc4ccccc43)C1)C2=O] |
| 45 | 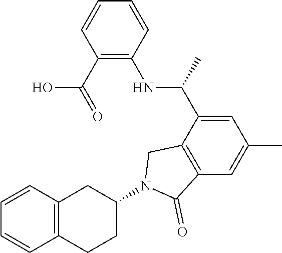 [SMILES: Cc1cc2c(c([C@@H](C)Nc3ccccc3C(=O)O)c1)CN([C@@H]1CCc3ccccc3C1)C2=O] |
| 46 | 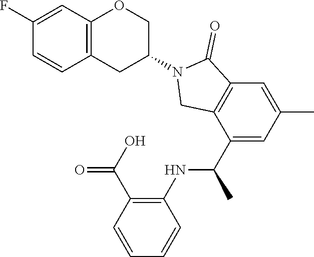 [SMILES: Cc1cc2c(c([C@@H](C)Nc3ccccc3C(=O)O)c1)CN([C@H]1COc3cc(F)ccc3C1)C2=O] |
| 47 | 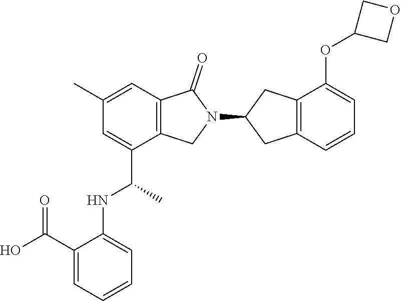 [SMILES: Cc1cc2c(c([C@H](C)Nc3ccccc3C(=O)O)c1)CN([C@@H]1Cc3cccc(OC4COC4)c3C1)C2=O] |
| 48 | 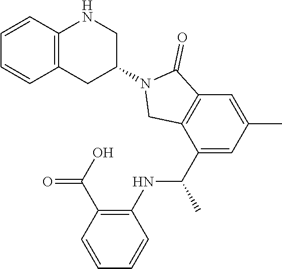 [SMILES: Cc1cc2c(c([C@H](C)Nc3ccccc3C(=O)O)c1)CN([C@H]1CNc3ccccc3C1)C2=O] |
| 49 | 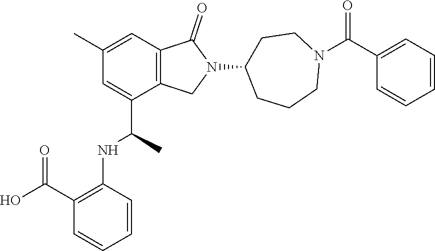 [SMILES: Cc1cc2c(c([C@@H](C)Nc3ccccc3C(=O)O)c1)CN([C@H]1CCCN(C(=O)c3ccccc3)CC1)C2=O] |
| 50 | 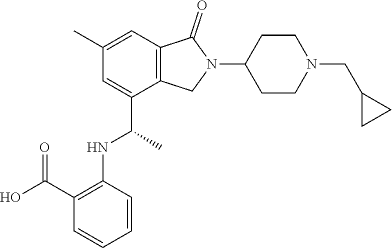 [SMILES: Cc1cc2c(c([C@H](C)Nc3ccccc3C(=O)O)c1)CN(C1CCN(CC3CC3)CC1)C2=O] |
| 51 | 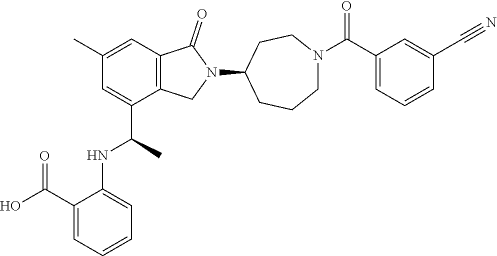 [SMILES: Cc1cc2c(c([C@@H](C)Nc3ccccc3C(=O)O)c1)CN([C@@H]1CCCN(C(=O)c3cccc(C#N)c3)CC1)C2=O] |
| 52 | 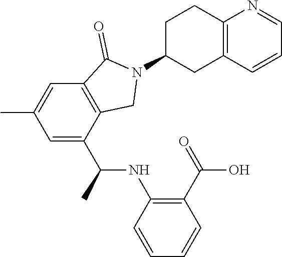 [SMILES: Cc1cc2c(c([C@H](C)Nc3ccccc3C(=O)O)c1)CN([C@H]1CCc3ncccc3C1)C2=O] |
| 53 | 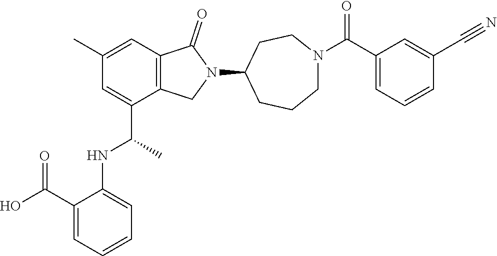 [SMILES: Cc1cc2c(c([C@H](C)Nc3ccccc3C(=O)O)c1)CN([C@@H]1CCCN(C(=O)c3cccc(C#N)c3)CC1)C2=O] |
| 54 | 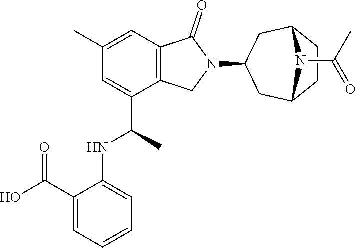 [SMILES: CC(=O)N1[C@@H]2CC[C@H]1C[C@H](N1Cc3c(cc(C)cc3[C@@H](C)Nc3ccccc3C(=O)O)C1=O)C2] |
| 55 | 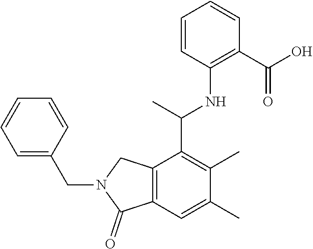 [SMILES: Cc1cc2c(c(C(C)Nc3ccccc3C(=O)O)c1C)CN(Cc1ccccc1)C2=O] |
| 56 | 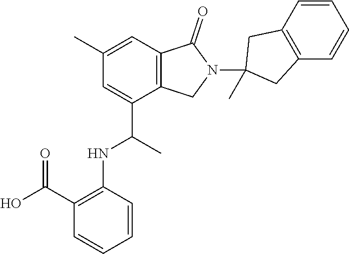 [SMILES: Cc1cc2c(c(C(C)Nc3ccccc3C(=O)O)c1)CN(C1(C)Cc3ccccc3C1)C2=O] |
| 57 | 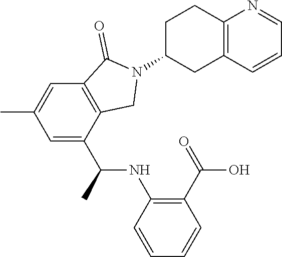 [SMILES: Cc1cc2c(c([C@H](C)Nc3ccccc3C(=O)O)c1)CN([C@@H]1CCc3ncccc3C1)C2=O] |
| 58 | 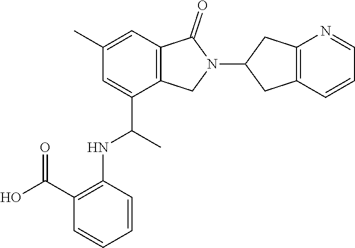 [SMILES: Cc1cc2c(c(C(C)Nc3ccccc3C(=O)O)c1)CN(C1Cc3cccnc3C1)C2=O] |
| 59 | 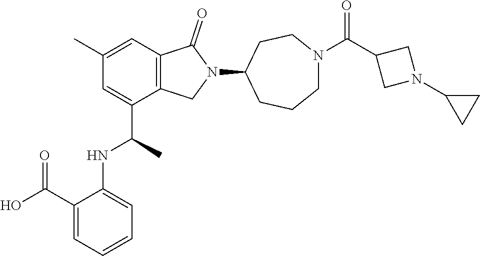 [SMILES: Cc1cc2c(c([C@@H](C)Nc3ccccc3C(=O)O)c1)CN([C@@H]1CCCN(C(=O)C3CN(C4CC4)C3)CC1)C2=O] |
| 60 | 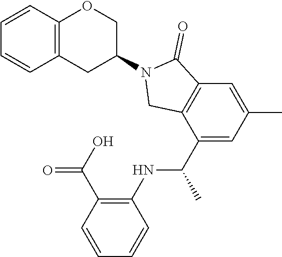 [SMILES: Cc1cc2c(c([C@H](C)Nc3ccccc3C(=O)O)c1)CN([C@@H]1COc3ccccc3C1)C2=O] |
| 61 | 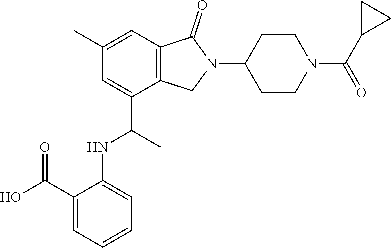 [SMILES: Cc1cc2c(c(C(C)Nc3ccccc3C(=O)O)c1)CN(C1CCN(C(=O)C3CC3)CC1)C2=O] |
| 62 | 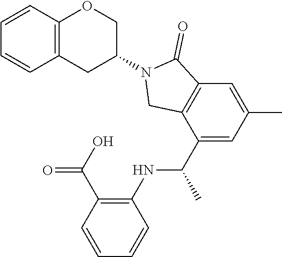 [SMILES: Cc1cc2c(c([C@H](C)Nc3ccccc3C(=O)O)c1)CN([C@H]1COc3ccccc3C1)C2=O] |
| 63 | 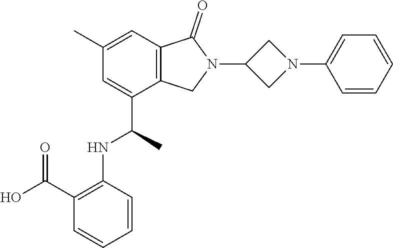 [SMILES: Cc1cc2c(c([C@@H](C)Nc3ccccc3C(=O)O)c1)CN(C1CN(c3ccccc3)C1)C2=O] |
| 64 | 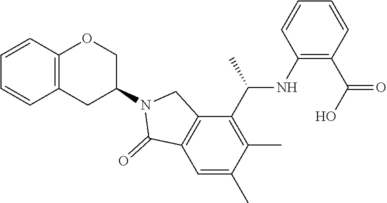 [SMILES: Cc1cc2c(c([C@H](C)Nc3ccccc3C(=O)O)c1C)CN([C@@H]1COc3ccccc3C1)C2=O] |
| 65 | 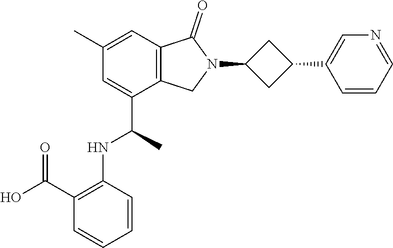 [SMILES: Cc1cc2c(c([C@@H](C)Nc3ccccc3C(=O)O)c1)CN([C@H]1C[C@H](c3cccnc3)C1)C2=O] |
| 66 | 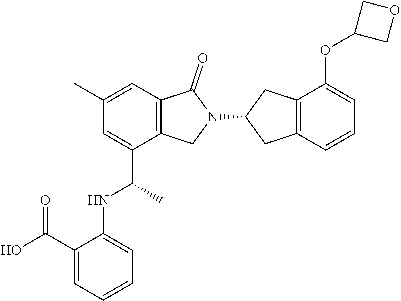 [SMILES: Cc1cc2c(c([C@H](C)Nc3ccccc3C(=O)O)c1)CN([C@H]1Cc3cccc(OC4COC4)c3C1)C2=O] |
| 67 | 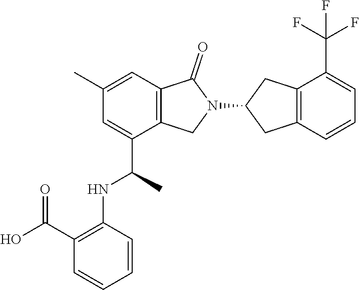 [SMILES: Cc1cc2c(c([C@@H](C)Nc3ccccc3C(=O)O)c1)CN([C@H]1Cc3cccc(C(F)(F)F)c3C1)C2=O] |
| 68 | 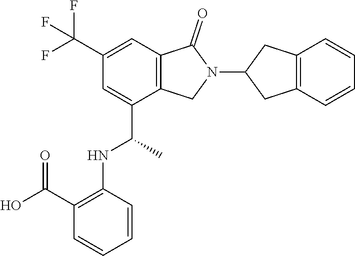 [SMILES: C[C@H](Nc1ccccc1C(=O)O)c1cc(C(F)(F)F)cc2c1CN(C1Cc3ccccc3C1)C2=O] |
| 69 | 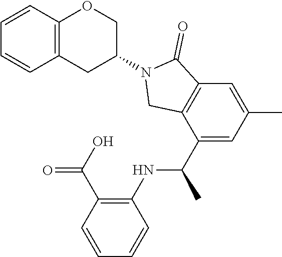 [SMILES: Cc1cc2c(c([C@@H](C)Nc3ccccc3C(=O)O)c1)CN([C@H]1COc3ccccc3C1)C2=O] |
| 70 | 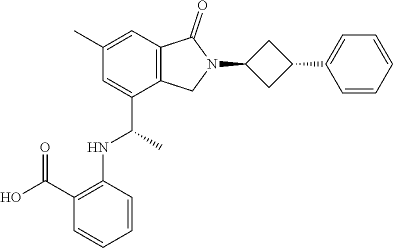 [SMILES: Cc1cc2c(c([C@H](C)Nc3ccccc3C(=O)O)c1)CN([C@H]1C[C@H](c3ccccc3)C1)C2=O] |
| 71 | 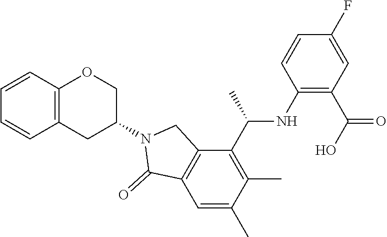 [SMILES: Cc1cc2c(c([C@H](C)Nc3ccc(F)cc3C(=O)O)c1C)CN([C@H]1COc3ccccc3C1)C2=O] |
| 72 | 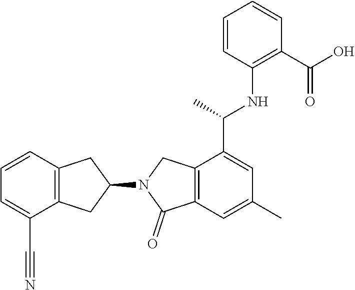 [SMILES: Cc1cc2c(c([C@H](C)Nc3ccccc3C(=O)O)c1)CN([C@@H]1Cc3cccc(C#N)c3C1)C2=O] |
| 73 | 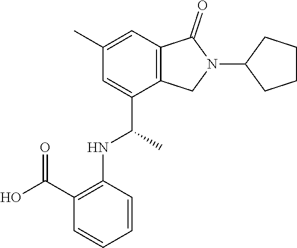 [SMILES: Cc1cc2c(c([C@H](C)Nc3ccccc3C(=O)O)c1)CN(C1CCCC1)C2=O] |
| 74 | 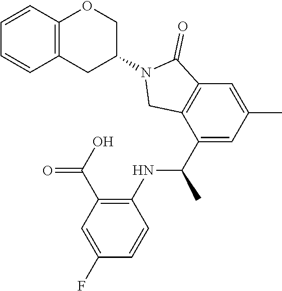 [SMILES: Cc1cc2c(c([C@@H](C)Nc3ccc(F)cc3C(=O)O)c1)CN([C@H]1COc3ccccc3C1)C2=O] |
| 75 | 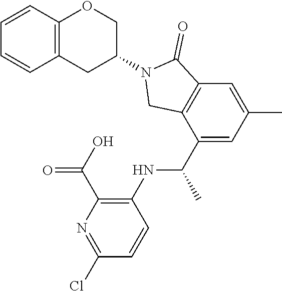 [SMILES: Cc1cc2c(c([C@H](C)Nc3ccc(Cl)nc3C(=O)O)c1)CN([C@H]1COc3ccccc3C1)C2=O] |
| 76 | 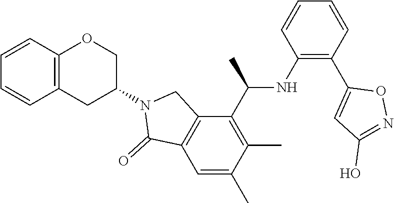 [SMILES: Cc1cc2c(c([C@@H](C)Nc3ccccc3-c3cc(O)no3)c1C)CN([C@H]1COc3ccccc3C1)C2=O] |
| 77 | 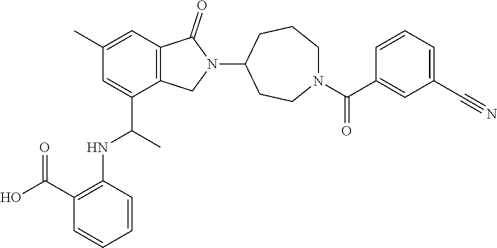 [SMILES: Cc1cc2c(c(C(C)Nc3ccccc3C(=O)O)c1)CN(C1CCCN(C(=O)c3cccc(C#N)c3)CC1)C2=O] |
| 78 | 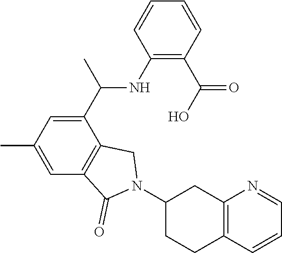 [SMILES: Cc1cc2c(c(C(C)Nc3ccccc3C(=O)O)c1)CN(C1CCc3cccnc3C1)C2=O] |
| 79 | 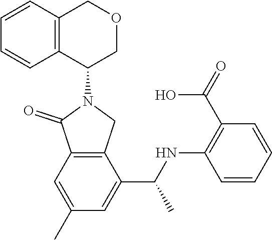 [SMILES: Cc1cc2c(c([C@@H](C)Nc3ccccc3C(=O)O)c1)CN([C@H]1COCc3ccccc31)C2=O] |
| 80 | 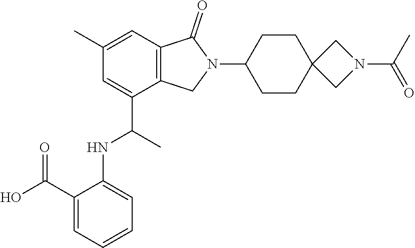 [SMILES: CC(=O)N1CC2(CCC(N3Cc4c(cc(C)cc4C(C)Nc4ccccc4C(=O)O)C3=O)CC2)C1] |
| 81 | 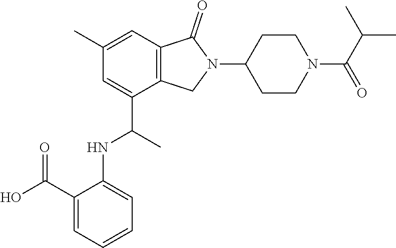 [SMILES: Cc1cc2c(c(C(C)Nc3ccccc3C(=O)O)c1)CN(C1CCN(C(=O)C(C)C)CC1)C2=O] |
| 82 | 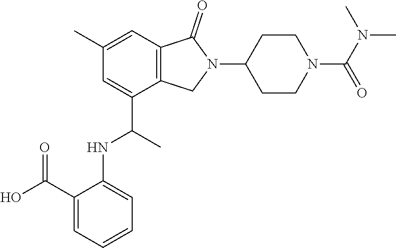 [SMILES: Cc1cc2c(c(C(C)Nc3ccccc3C(=O)O)c1)CN(C1CCN(C(=O)N(C)C)CC1)C2=O] |
| 83 | 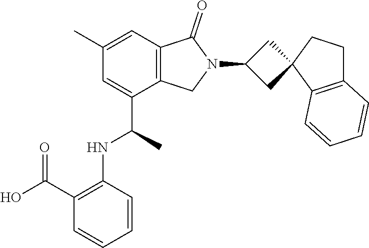 [SMILES: Cc1cc2c(c([C@@H](C)Nc3ccccc3C(=O)O)c1)CN([C@H]1C[C@@]3(CCc4ccccc43)C1)C2=O] |
| 84 | 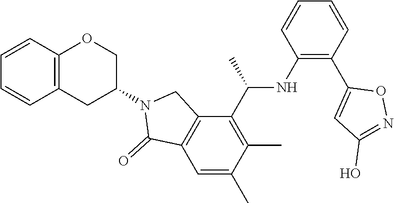 [SMILES: Cc1cc2c(c([C@H](C)Nc3ccccc3-c3cc(O)no3)c1C)CN([C@H]1COc3ccccc3C1)C2=O] |
| 85 | 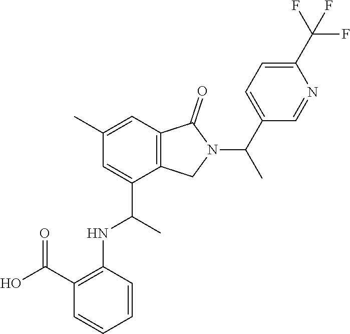 [SMILES: Cc1cc2c(c(C(C)Nc3ccccc3C(=O)O)c1)CN(C(C)c1ccc(C(F)(F)F)nc1)C2=O] |
| 86 | 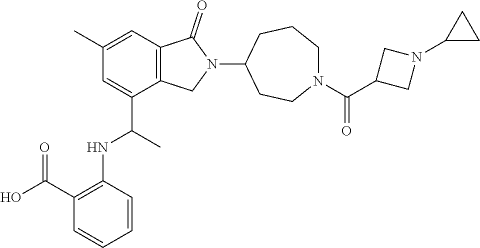 [SMILES: Cc1cc2c(c(C(C)Nc3ccccc3C(=O)O)c1)CN(C1CCCN(C(=O)C3CN(C4CC4)C3)CC1)C2=O] |
| 87 | 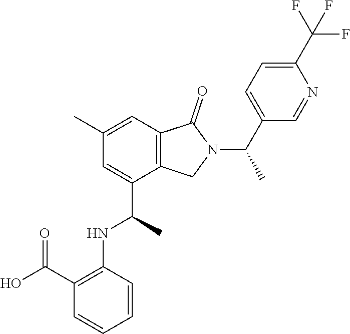 [SMILES: Cc1cc2c(c([C@@H](C)Nc3ccccc3C(=O)O)c1)CN([C@@H](C)c1ccc(C(F)(F)F)nc1)C2=O] |
| 88 | 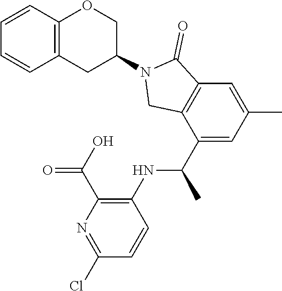 [SMILES: Cc1cc2c(c([C@@H](C)Nc3ccc(Cl)nc3C(=O)O)c1)CN([C@@H]1COc3ccccc3C1)C2=O] |
| 89 | 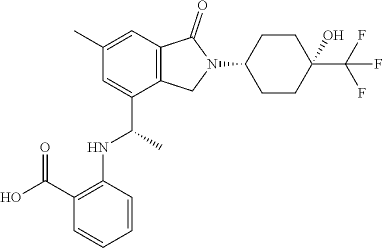 [SMILES: Cc1cc2c(c([C@H](C)Nc3ccccc3C(=O)O)c1)CN([C@H]1CC[C@](O)(C(F)(F)F)CC1)C2=O] |
| 90 | 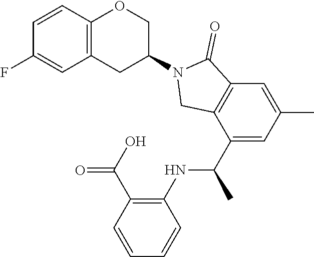 [SMILES: Cc1cc2c(c([C@@H](C)Nc3ccccc3C(=O)O)c1)CN([C@@H]1COc3ccc(F)cc3C1)C2=O] |
| 91 | 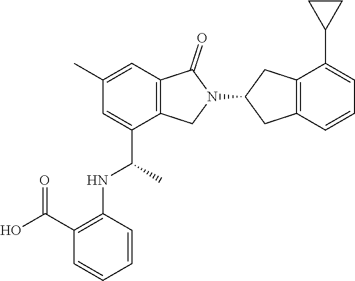 [SMILES: Cc1cc2c(c([C@H](C)Nc3ccccc3C(=O)O)c1)CN([C@H]1Cc3cccc(C4CC4)c3C1)C2=O] |
| 92 | 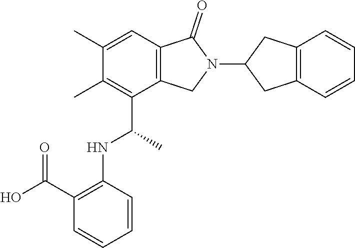 [SMILES: Cc1cc2c(c([C@H](C)Nc3ccccc3C(=O)O)c1C)CN(C1Cc3ccccc3C1)C2=O] |
| 93 | 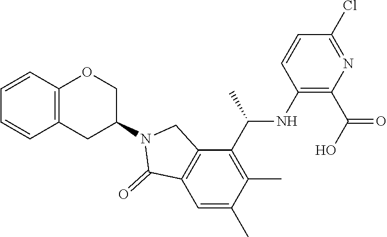 [SMILES: Cc1cc2c(c([C@H](C)Nc3ccc(Cl)nc3C(=O)O)c1C)CN([C@@H]1COc3ccccc3C1)C2=O] |
| 94 | 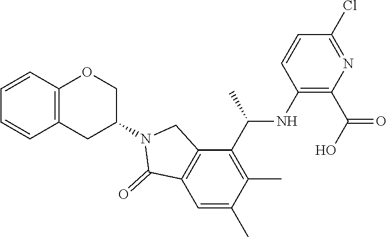 [SMILES: Cc1cc2c(c([C@H](C)Nc3ccc(Cl)nc3C(=O)O)c1C)CN([C@H]1COc3ccccc3C1)C2=O] |
| 95 | 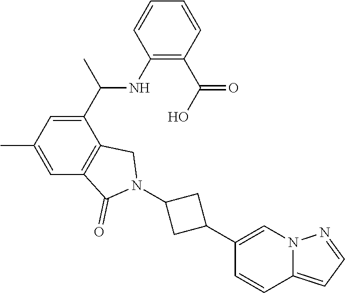 [SMILES: Cc1cc2c(c(C(C)Nc3ccccc3C(=O)O)c1)CN(C1CC(c3ccc4ccnn4c3)C1)C2=O] |
| 96 | 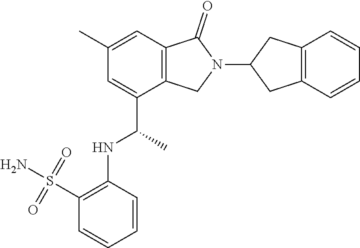 [SMILES: Cc1cc2c(c([C@H](C)Nc3ccccc3S(N)(=O)=O)c1)CN(C1Cc3ccccc3C1)C2=O] |
| 97 | 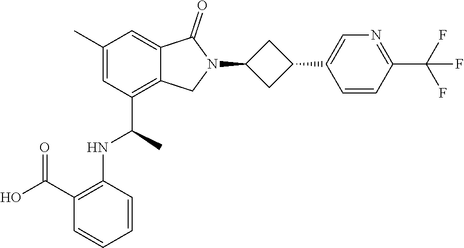 [SMILES: Cc1cc2c(c([C@@H](C)Nc3ccccc3C(=O)O)c1)CN([C@H]1C[C@H](c3ccc(C(F)(F)F)nc3)C1)C2=O] |
| 98 | 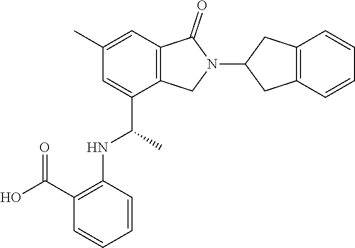 [SMILES: Cc1cc2c(c([C@H](C)Nc3ccccc3C(=O)O)c1)CN(C1Cc3ccccc3C1)C2=O] |
| 99 | 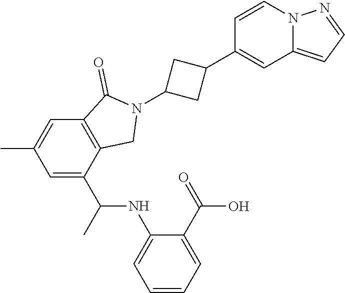 [SMILES: Cc1cc2c(c(C(C)Nc3ccccc3C(=O)O)c1)CN(C1CC(c3ccn4nccc4c3)C1)C2=O] |
| 100 | 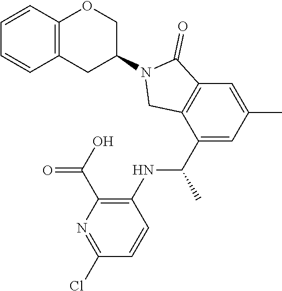 [SMILES: Cc1cc2c(c([C@H](C)Nc3ccc(Cl)nc3C(=O)O)c1)CN([C@@H]1COc3ccccc3C1)C2=O] |
| 101 | 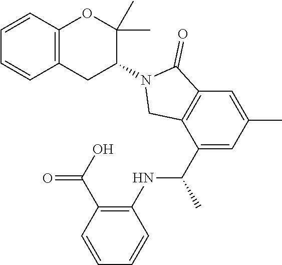 [SMILES: Cc1cc2c(c([C@H](C)Nc3ccccc3C(=O)O)c1)CN([C@@H]1Cc3ccccc3OC1(C)C)C2=O] |
| 102 | 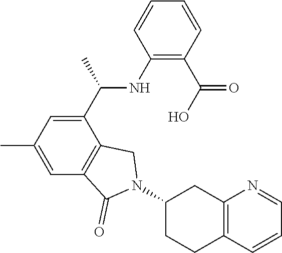 [SMILES: Cc1cc2c(c([C@H](C)Nc3ccccc3C(=O)O)c1)CN([C@H]1CCc3cccnc3C1)C2=O] |
| 103 | 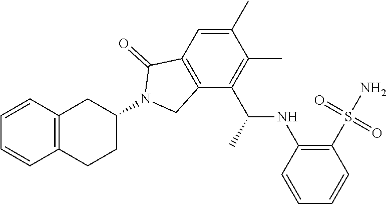 [SMILES: Cc1cc2c(c([C@@H](C)Nc3ccccc3S(N)(=O)=O)c1C)CN([C@@H]1CCc3ccccc3C1)C2=O] |
| 104 | 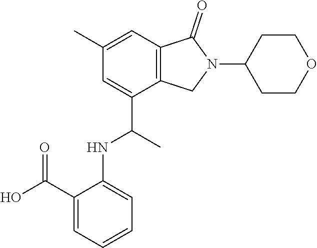 [SMILES: Cc1cc2c(c(C(C)Nc3ccccc3C(=O)O)c1)CN(C1CCOCC1)C2=O] |
| 105 | 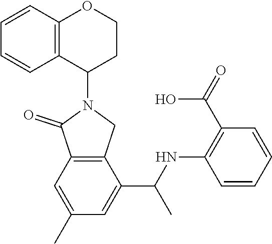 [SMILES: Cc1cc2c(c(C(C)Nc3ccccc3C(=O)O)c1)CN(C1CCOc3ccccc31)C2=O] |
| 106 | 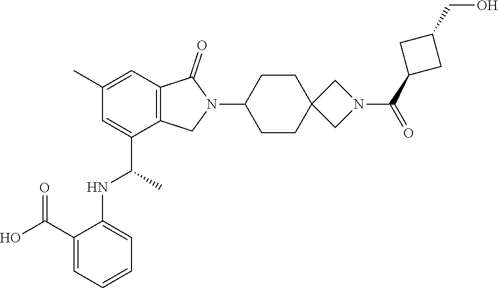 [SMILES: Cc1cc2c(c([C@H](C)Nc3ccccc3C(=O)O)c1)CN(C1CCC3(CC1)CN(C(=O)[C@H]1C[C@H](CO)C1)C3)C2=O] |
| 107 | 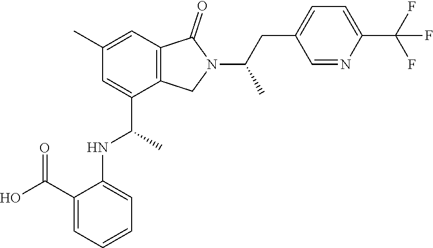 [SMILES: Cc1cc2c(c([C@H](C)Nc3ccccc3C(=O)O)c1)CN([C@@H](C)Cc1ccc(C(F)(F)F)nc1)C2=O] |
| 108 | 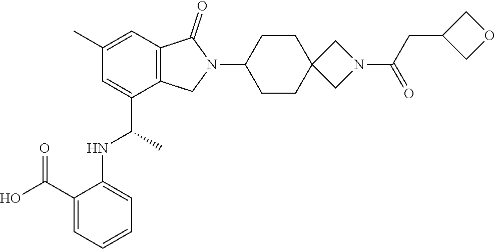 [SMILES: Cc1cc2c(c([C@H](C)Nc3ccccc3C(=O)O)c1)CN(C1CCC3(CC1)CN(C(=O)CC1COC1)C3)C2=O] |
| 109 | 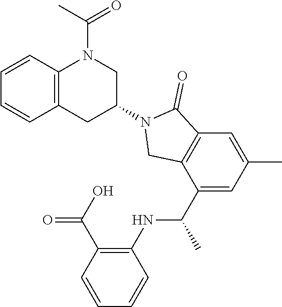 [SMILES: CC(=O)N1C[C@H](N2Cc3c(cc(C)cc3[C@H](C)Nc3ccccc3C(=O)O)C2=O)Cc2ccccc21] |
| 110 | 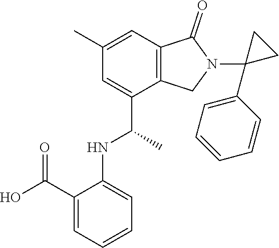 [SMILES: Cc1cc2c(c([C@H](C)Nc3ccccc3C(=O)O)c1)CN(C1(c3ccccc3)CC1)C2=O] |
| 111 | 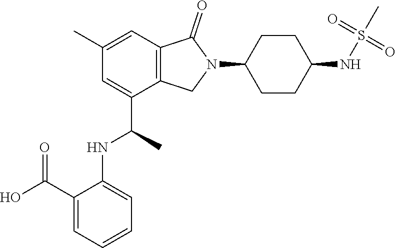 [SMILES: Cc1cc2c(c([C@@H](C)Nc3ccccc3C(=O)O)c1)CN([C@H]1CC[C@@H](NS(C)(=O)=O)CC1)C2=O] |
| 112 | 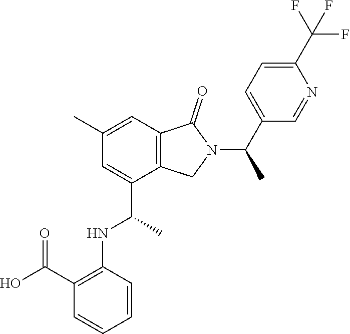 [SMILES: Cc1cc2c(c([C@H](C)Nc3ccccc3C(=O)O)c1)CN([C@H](C)c1ccc(C(F)(F)F)nc1)C2=O] |
| 113 | 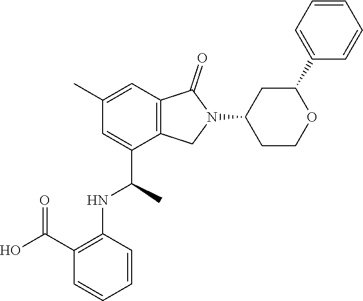 [SMILES: Cc1cc2c(c([C@@H](C)Nc3ccccc3C(=O)O)c1)CN([C@H]1CCO[C@@H](c3ccccc3)C1)C2=O] |
| 114 | 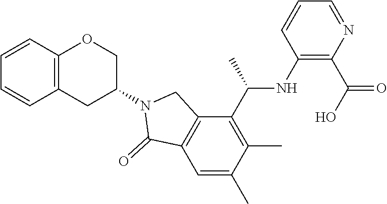 [SMILES: Cc1cc2c(c([C@H](C)Nc3cccnc3C(=O)O)c1C)CN([C@H]1COc3ccccc3C1)C2=O] |
| 115 | 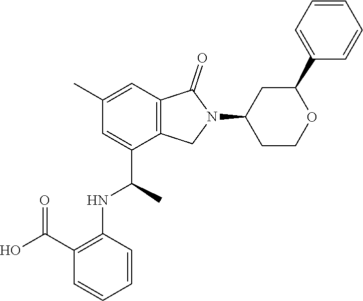 [SMILES: Cc1cc2c(c([C@@H](C)Nc3ccccc3C(=O)O)c1)CN([C@@H]1CCO[C@H](c3ccccc3)C1)C2=O] |
| 116 | 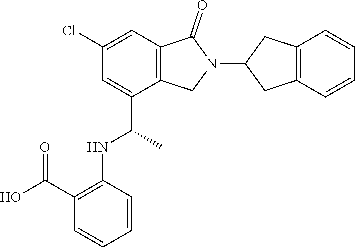 [SMILES: C[C@H](Nc1ccccc1C(=O)O)c1cc(Cl)cc2c1CN(C1Cc3ccccc3C1)C2=O] |
| 117 | 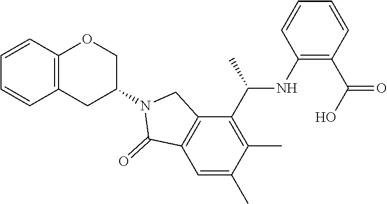 [SMILES: Cc1cc2c(c([C@H](C)Nc3ccccc3C(=O)O)c1C)CN([C@H]1COc3ccccc3C1)C2=O] |
| 118 | 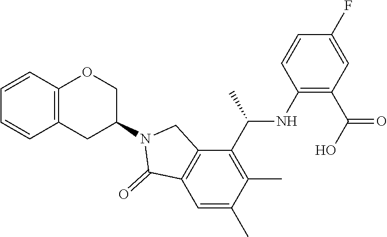 [SMILES: Cc1cc2c(c([C@H](C)Nc3ccc(F)cc3C(=O)O)c1C)CN([C@@H]1COc3ccccc3C1)C2=O] |
| 119 | 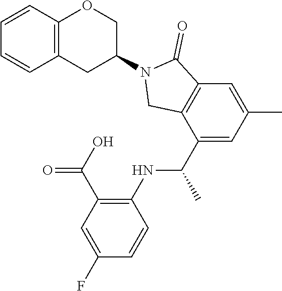 [SMILES: Cc1cc2c(c([C@H](C)Nc3ccc(F)cc3C(=O)O)c1)CN([C@@H]1COc3ccccc3C1)C2=O] |
| 120 | 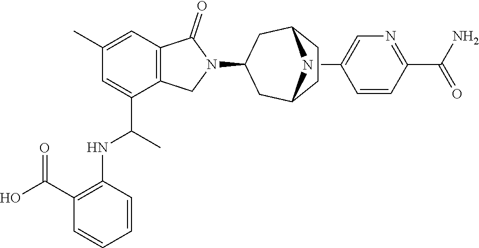 [SMILES: Cc1cc2c(c(C(C)Nc3ccccc3C(=O)O)c1)CN([C@@H]1C[C@H]3CC[C@@H](C1)N3c1ccc(C(N)=O)nc1)C2=O] |
| 121 | 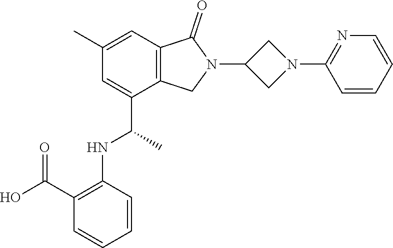 [SMILES: Cc1cc2c(c([C@H](C)Nc3ccccc3C(=O)O)c1)CN(C1CN(c3ccccn3)C1)C2=O] |
| 122 | 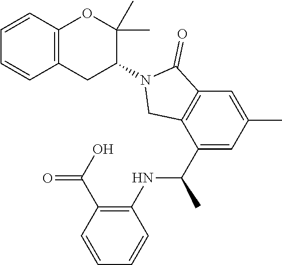 [SMILES: Cc1cc2c(c([C@@H](C)Nc3ccccc3C(=O)O)c1)CN([C@@H]1Cc3ccccc3OC1(C)C)C2=O] |
| 123 | 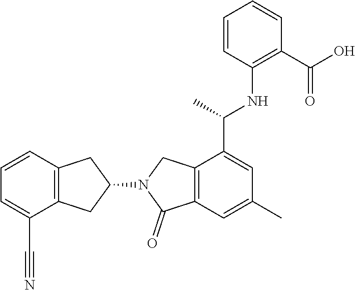 [SMILES: Cc1cc2c(c([C@H](C)Nc3ccccc3C(=O)O)c1)CN([C@H]1Cc3cccc(C#N)c3C1)C2=O] |
| 124 | 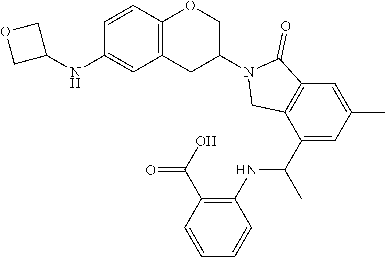 [SMILES: Cc1cc2c(c(C(C)Nc3ccccc3C(=O)O)c1)CN(C1COc3ccc(NC4COC4)cc3C1)C2=O] |
| 125 | 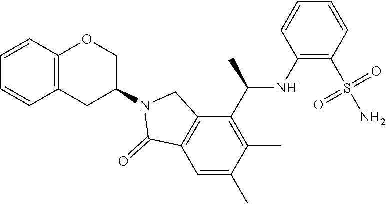 [SMILES: Cc1cc2c(c([C@@H](C)Nc3ccccc3S(N)(=O)=O)c1C)CN([C@@H]1COc3ccccc3C1)C2=O] |
| 126 | 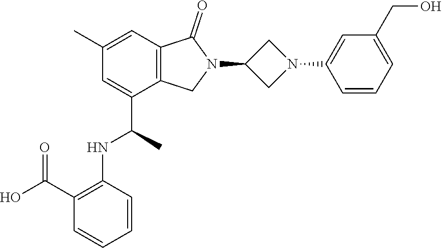 |
| 127 | 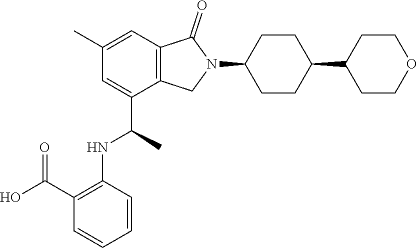 [SMILES: Cc1cc2c(c([C@@H](C)Nc3ccccc3C(=O)O)c1)CN([C@H]1CC[C@@H](C3CCOCC3)CC1)C2=O] |
| 128 | 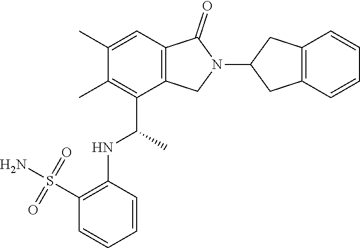 [SMILES: Cc1cc2c(c([C@H](C)Nc3ccccc3S(N)(=O)=O)c1C)CN(C1Cc3ccccc3C1)C2=O] |
| 129 | 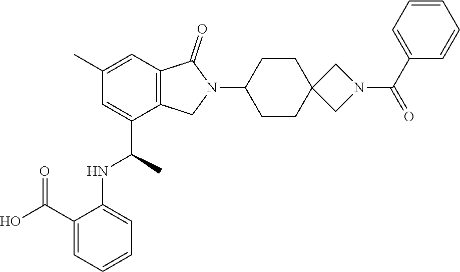 [SMILES: Cc1cc2c(c([C@@H](C)Nc3ccccc3C(=O)O)c1)CN(C1CCC3(CC1)CN(C(=O)c1ccccc1)C3)C2=O] |
| 130 | 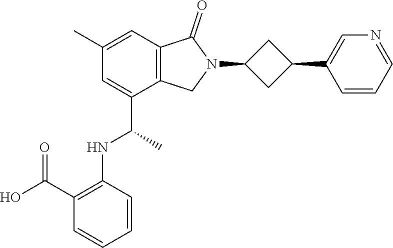 [SMILES: Cc1cc2c(c([C@H](C)Nc3ccccc3C(=O)O)c1)CN([C@H]1C[C@@H](c3cccnc3)C1)C2=O] |
| 131 | 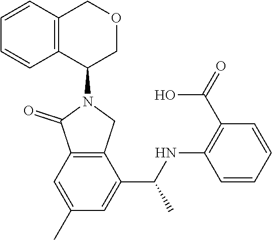 [SMILES: Cc1cc2c(c([C@@H](C)Nc3ccccc3C(=O)O)c1)CN([C@@H]1COCc3ccccc31)C2=O] |
| 132 | 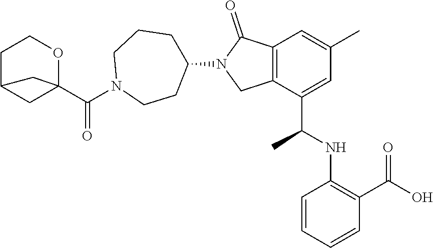 [SMILES: Cc1cc2c(c([C@H](C)Nc3ccccc3C(=O)O)c1)CN([C@H]1CCCN(C(=O)C34CC(CCO3)C4)CC1)C2=O] |
| 133 | 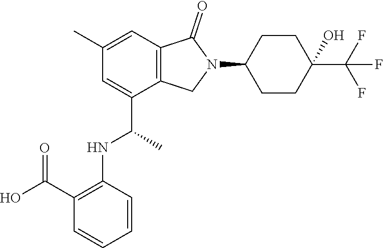 [SMILES: Cc1cc2c(c([C@H](C)Nc3ccccc3C(=O)O)c1)CN([C@H]1CC[C@@](O)(C(F)(F)F)CC1)C2=O] |
| 134 | 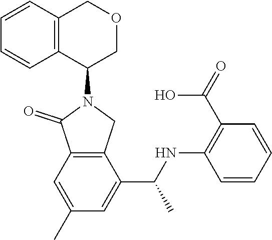 [SMILES: Cc1cc2c(c([C@@H](C)Nc3ccccc3C(=O)O)c1)CN([C@@H]1COCc3ccccc31)C2=O] |
| 135 | 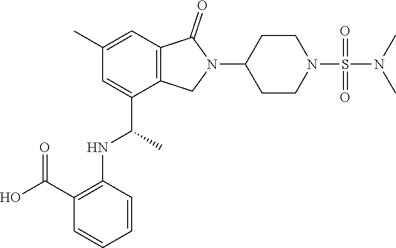 [SMILES: Cc1cc2c(c([C@H](C)Nc3ccccc3C(=O)O)c1)CN(C1CCN(S(=O)(=O)N(C)C)CC1)C2=O] |
| 136 | 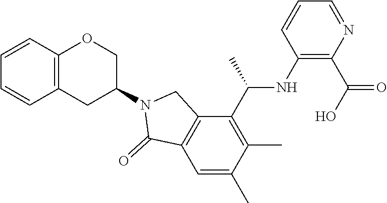 [SMILES: Cc1cc2c(c([C@H](C)Nc3cccnc3C(=O)O)c1C)CN([C@@H]1COc3ccccc3C1)C2=O] |
| 137 | 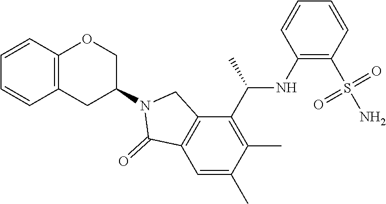 [SMILES: Cc1cc2c(c([C@H](C)Nc3ccccc3S(N)(=O)=O)c1C)CN([C@@H]1COc3ccccc3C1)C2=O] |
| 138 | 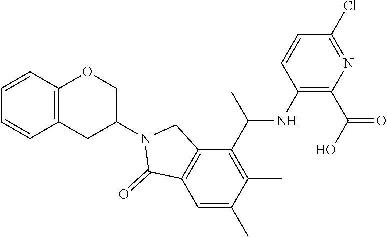 [SMILES: Cc1cc2c(c(C(C)Nc3ccc(Cl)nc3C(=O)O)c1C)CN(C1COc3ccccc3C1)C2=O] |
| 139 | 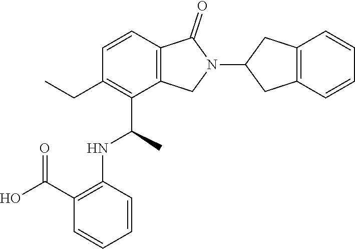 [SMILES: CCc1ccc2c(c1[C@@H](C)Nc1ccccc1C(=O)O)CN(C1Cc3ccccc3C1)C2=O] |
| 140 | 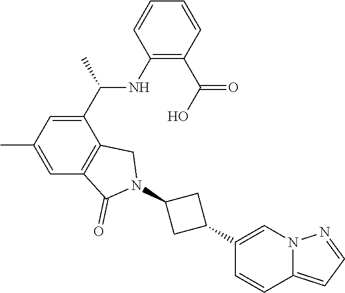 [SMILES: Cc1cc2c(c([C@H](C)Nc3ccccc3C(=O)O)c1)CN([C@H]1C[C@H](c3ccc4ccnn4c3)C1)C2=O] |
| 141 | 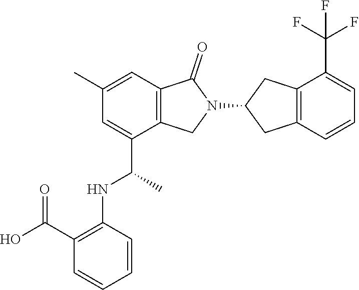 [SMILES: Cc1cc2c(c([C@H](C)Nc3ccccc3C(=O)O)c1)CN([C@H]1Cc3cccc(C(F)(F)F)c3C1)C2=O] |
| 142 | 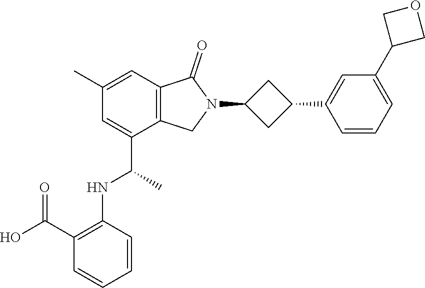 [SMILES: Cc1cc2c(c([C@H](C)Nc3ccccc3C(=O)O)c1)CN([C@H]1C[C@H](c3cccc(C4COC4)c3)C1)C2=O] |
| 143 | 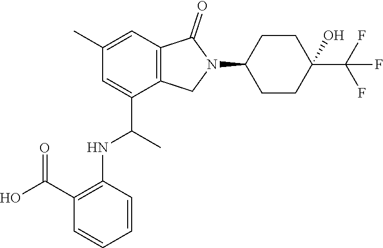 [SMILES: Cc1cc2c(c(C(C)Nc3ccccc3C(=O)O)c1)CN([C@H]1CC[C@@](O)(C(F)(F)F)CC1)C2=O] |
| 144 | 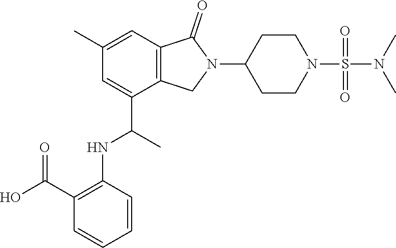 [SMILES: Cc1cc2c(c(C(C)Nc3ccccc3C(=O)O)c1)CN(C1CCN(S(=O)(=O)N(C)C)CC1)C2=O] |
| 145 | 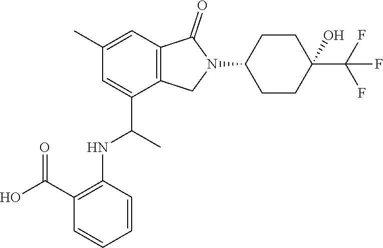 [SMILES: Cc1cc2c(c(C(C)Nc3ccccc3C(=O)O)c1)CN([C@H]1CC[C@](O)(C(F)(F)F)CC1)C2=O] |
| 146 | 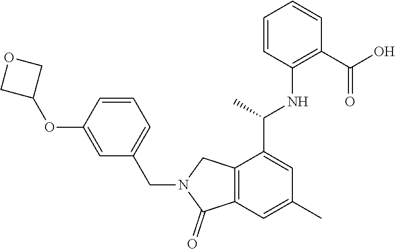 [SMILES: Cc1cc2c(c([C@H](C)Nc3ccccc3C(=O)O)c1)CN(Cc1cccc(OC3COC3)c1)C2=O] |
| 147 | 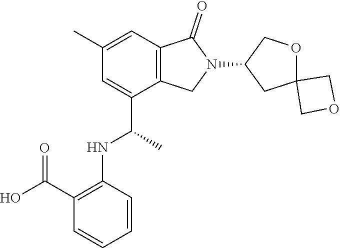 [SMILES: Cc1cc2c(c([C@H](C)Nc3ccccc3C(=O)O)c1)CN([C@@H]1COC3(COC3)C1)C2=O] |
| 148 | 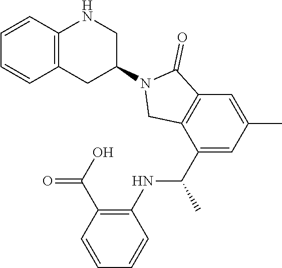 [SMILES: Cc1cc2c(c([C@H](C)Nc3ccccc3C(=O)O)c1)CN([C@@H]1CNc3ccccc3C1)C2=O] |
| 149 | 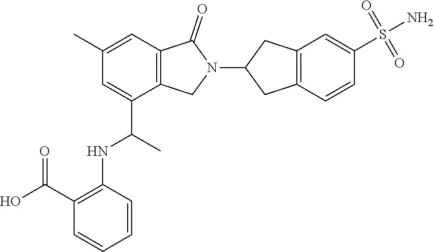 [SMILES: Cc1cc2c(c(C(C)Nc3ccccc3C(=O)O)c1)CN(C1Cc3ccc(S(N)(=O)=O)cc3C1)C2=O] |
| 150 | 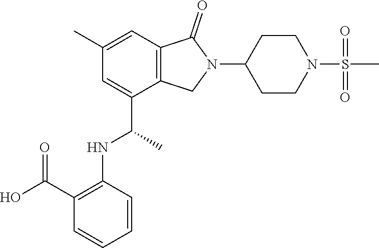 [SMILES: Cc1cc2c(c([C@H](C)Nc3ccccc3C(=O)O)c1)CN(C1CCN(S(C)(=O)=O)CC1)C2=O] |
| 151 | 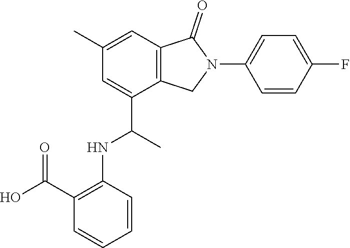 [SMILES: Cc1cc2c(c(C(C)Nc3ccccc3C(=O)O)c1)CN(c1ccc(F)cc1)C2=O] |
| 152 | 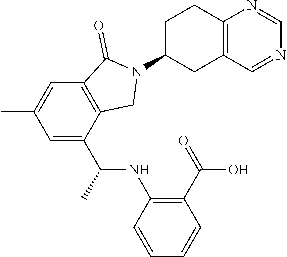 [SMILES: Cc1cc2c(c([C@@H](C)Nc3ccccc3C(=O)O)c1)CN([C@H]1CCc3ncncc3C1)C2=O] |
| 153 | 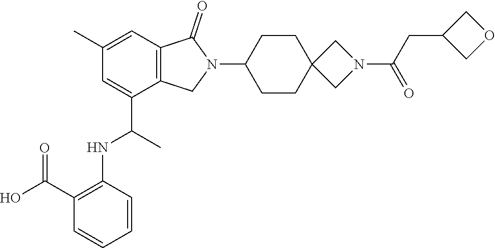 [SMILES: Cc1cc2c(c(C(C)Nc3ccccc3C(=O)O)c1)CN(C1CCC3(CC1)CN(C(=O)CC1COC1)C3)C2=O] |
| 154 | 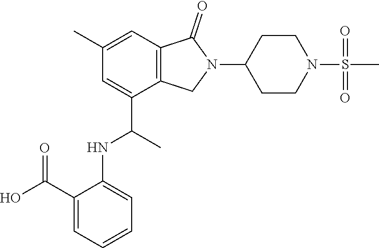 [SMILES: Cc1cc2c(c(C(C)Nc3ccccc3C(=O)O)c1)CN(C1CCN(S(C)(=O)=O)CC1)C2=O] |
| 155 | 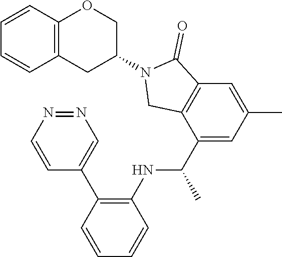 [SMILES: Cc1cc2c(c([C@H](C)Nc3ccccc3-c3ccnnc3)c1)CN([C@H]1COc3ccccc3C1)C2=O] |

### Pharmaceutical Compositions

Some embodiments provide a pharmaceutical composition comprising a compound of Formula (I), or a pharmaceutically acceptable salt thereof, and one or more pharmaceutically acceptable excipients.

### Methods of Treatment

A “PI3Kα inhibitor” as used herein (e.g., compounds of Formula (I) and pharmaceutically acceptable salts thereof) includes any compound exhibiting PI3Kα inactivation activity (e.g., inhibiting or decreasing). In some embodiments, a PI3Kα inhibitor can be selective for a PI3Kα having one or more mutations.

The ability of test compounds to act as inhibitors of PI3Kα may be demonstrated by assays known in the art. The activity of the compounds and compositions provided herein as PI3Kα inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labeling the compound prior to binding, isolating the compound/kinase complex and determining the amount of radio label bound, or by running a competition experiment where new compounds are incubated with the kinase bound to known radio ligands.

Potency of a PI3Kα inhibitor as provided herein can be determined by EC₅₀ value. A compound with a lower EC₅₀ value, as determined under substantially similar conditions, is a more potent inhibitor relative to a compound with a higher EC₅₀ value.

Potency of a PI3Kα inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a PI3Kα-dependent phosphorylation level, in vitro or in vivo.

The selectivity between wild type PI3Kα and PI3Kα containing one or more mutations as described herein can also be measured using in vitro assays such as surface plasmon resonance and fluorence-based binding assays, and cellular assays such as the levels of pAKT, a biomarker of PI3Kα activity, and/or proliferation assays where cell proliferation is dependent on mutant PI3Kα kinase activity.

In some embodiments, the compounds provided herein can exhibit potent and selective inhibition of PI3Kα. For example, the compounds provided herein can bind to the helical phosphatidylinositol kinase homology domain catalytic domain of PI3Kα. In some embodiments, the compounds provided herein can exhibit nanomolar potency against a PI3Kα kinase including one or more mutations, for example, the mutations in Table 1.

In some embodiments, the compounds of Formula (I), or a pharmaceutically acceptable salt thereof, can selectively target PI3Kα. For example, a compound of Formula (I), or a pharmaceutically acceptable salt thereof, can selectively target PI3Kα over another kinase or non-kinase target.

In some embodiments, a compound of Formula (I), or a pharmaceutically acceptable salt thereof, can exhibit greater inhibition of PI3Kα containing one or more mutations as described herein (e.g., one or more mutations as described in Table 1) relative to inhibition of wild type PI3Kα. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 1,000-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 10,000-fold greater inhibition of PI3Kα having a combination of mutations described herein relative to inhibition of wild type PI3Kα.

In some embodiments, a compound of Formula (I), or a pharmaceutically acceptable salt thereof, can exhibit from about 2-fold to about 10-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 10-fold to about 100-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 100-fold to about 1,000-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 1000-fold to about 10,000-fold greater inhibition of PI3Kα containing one or more mutations as described herein relative to inhibition of wild type PI3Kα.

Compounds of Formula (I), or pharmaceutically acceptable salts thereof, are useful for treating diseases which can be treated with a PI3Kα inhibitor, such as PI3Kα-associated diseases, e.g., proliferative disorders such as cancers, including hematological cancers and solid tumors (e.g., advanced or metastatic solid tumors).

In some embodiments, the subject has been identified or diagnosed as having a cancer with a dysregulation of a PIK3CA gene, a PI3Kα protein, or expression or activity, or level of any of the same (a PI3Kα-associated cancer), for example, as determined using a regulatory agency-approved, e.g., FDA-approved, assay or kit. In some embodiments, the subject has a tumor that is positive for a dysregulation of a PIK3CA gene, a PI3Kα protein, or expression or activity, or level of any of the same (e.g., as determined using a regulatory agency-approved assay or kit). For example, the subject has a tumor that is positive for a mutation as described in Table 1. The subject can be a subject with a tumor(s) that is positive for a dysregulation of a PIK3CA gene, a PI3Kα protein, or expression or activity, or level of any of the same (e.g., identified as positive using a regulatory agency-approved, e.g., FDA-approved, assay or kit). The subject can be a subject whose tumors have a dysregulation of a PIK3CA gene, a PI3Kα protein, or expression or activity, or a level of the same (e.g., where the tumor is identified as such using a regulatory agency-approved, e.g., FDA-approved, kit or assay). In some embodiments, the subject is suspected of having a PI3Kα-associated cancer. In some embodiments, the subject has a clinical record indicating that the subject has a tumor that has a dysregulation of a PIK3CA gene, a PI3Kα protein, or expression or activity, or level of any of the same (and optionally the clinical record indicates that the subject should be treated with any of the compositions provided herein).

In certain embodiments, compounds of Formula (I), or pharmaceutically acceptable salts thereof, are useful for preventing diseases as defined herein such as cancer. The term “preventing” as used herein means to delay the onset, recurrence or spread, in whole or in part, of the disease as described herein, or a symptom thereof.

The term “PI3Kα-associated disease” as used herein refers to diseases associated with or having a dysregulation of a PIK3CA gene, a PI3Kα protein, or the expression or activity or level of any (e.g., one or more) of the same (e.g., any of the types of dysregulation of a PIK3CA gene, or a PI3Kα protein, or the expression or activity or level of any of the same described herein). Non-limiting examples of a PI3Kα-associated disease include, for example, proliferative disorders such as cancer (e.g., PI3Kα-associated cancer).

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation (e.g., a mutation in a PIK3CA gene that results in the expression of a PI3Kα that includes a deletion of at least one amino acid as compared to a wild type PI3Kα, a mutation in a PIK3CA gene that results in the expression of PI3Kα with one or more point mutations as compared to a wild type PI3Kα, a mutation in a PIK3CA gene that results in the expression of PI3Kα with at least one inserted amino acid as compared to a wild type PI3Kα, a gene duplication that results in an increased level of PI3Kα in a cell, or a mutation in a regulatory sequence (e.g., a promoter and/or enhancer) that results in an increased level of PI3Kα in a cell), an alternative spliced version of PI3Kα mRNA that results in PI3Kα having a deletion of at least one amino acid in the PI3Kα as compared to the wild type PI3Kα), or increased expression (e.g., increased levels) of a wild type PI3Kα in a mammalian cell due to aberrant cell signaling and/or dysregulated autocrine/paracrine signaling (e.g., as compared to a control non-cancerous cell). As another example, a dysregulation of a PIK3CA gene, a PI3Kα protein, or expression or activity, or level of any of the same, can be a mutation in a PIK3CA gene that encodes a PI3Kα that is constitutively active or has increased activity as compared to a protein encoded by a PIK3CA gene that does not include the mutation. Non-limiting examples of PI3Kα point mutations/substitutions/insertions/deletions are described in Table 1.

The term “wild type” describes a nucleic acid (e.g., a PIK3CA gene or a PI3Kα mRNA) or protein (e.g., a PI3Kα) sequence that is typically found in a subject that does not have a disease related to the reference nucleic acid or protein.

The term “wild type PI3Kα” or “wild-type PI3Kα” describes a normal PI3Kα nucleic acid (e.g., a PIK3CA or PI3Kα mRNA) or protein that is found in a subject that does not have a PI3Kα-associated disease, e.g., a PI3Kα-associated cancer (and optionally also does not have an increased risk of developing a PI3Kα-associated disease and/or is not suspected of having a PI3Kα-associated disease), or is found in a cell or tissue from a subject that does not have a PI3Kα-associated disease, e.g., a PI3Kα-associated cancer (and optionally also does not have an increased risk of developing a PI3Kα-associated disease and/or is not suspected of having a PI3Kα-associated disease).

Provided herein is a method of treating cancer (e.g., a PI3Kα-associated cancer) in a subject in need thereof, comprising administering to the subject a therapeutically effective amount of a compound of Formula (I), or a pharmaceutically acceptable salt thereof, or a pharmaceutical composition thereof. For example, provided herein are methods for treating PI3Kα-associated cancer in a subject in need thereof, comprising a) detecting a dysregulation of PIK3CA gene, a PI3Kα protein, or the expression or activity or level of any of the same in a sample from the subject; and b) administering a therapeutically effective amount of a compound of Formula (I), or a pharmaceutically acceptable salt thereof. In some embodiments, the dysregulation of a PIK3CA gene, a PI3Kα protein, or the expression or activity or level of any of the same includes one or more a PI3Kα protein substitutions/point mutations/insertions. Non-limiting examples of PI3Kα protein substitutions/insertions/deletions are described in Table 1.

In some embodiments, the PI3Kα protein substitution/insertion/deletion is selected from the group consisting of E542A, E542G, E542K, E542Q, E542V, E545A, E545D, E545G, E545K, E545Q, M10431, M1043L, M1043T, M1043V, H1047L, H1047Q, H1047R, H1047Y, G1049R, and combinations thereof. In some embodiments, the PI3Kα protein substitution/insertion/deletion is H1047X, where X is any amino acid other than H. In some embodiments, the PI3Kα protein substitution/insertion/deletion is E542X, where X is any amino acid other than E. In some embodiments, the PI3Kα protein substitution/insertion/deletion is E545X, where X is any amino acid other than E.

In some embodiments, the dysregulation of a PIK3CA gene, a PI3Kα protein, or expression or activity or level of any of the same, includes a splice variation in a PI3Kα mRNA which results in an expressed protein that is an alternatively spliced variant of PI3Kα having at least one residue deleted (as compared to the wild type PI3Kα protein) resulting in a constitutive activity of a PI3Kα protein domain.

In some embodiments, the dysregulation of a PIK3CA gene, a PI3Kα protein, or expression or activity or level of any of the same, includes at least one point mutation in a PIK3CA gene that results in the production of a PI3Kα protein that has one or more amino acid substitutions or insertions or deletions in a PIK3CA gene that results in the production of a PI3Kα protein that has one or more amino acids inserted or removed, as compared to the wild type PI3Kα protein. In some cases, the resulting mutant PI3Kα protein has increased activity, as compared to a wild type PI3Kα protein or a PI3Kα protein not including the same mutation. In some embodiments, the compounds described herein selectively inhibit the resulting mutant PI3Kα protein relative to a wild type PI3Kα protein or a PI3Kα protein not including the same mutation.

In some embodiments of any of the methods or uses described herein, the cancer (e.g., PI3Kα-associated cancer) is selected from a hematological cancer and a solid tumor.

In some embodiments of any of the methods or uses described herein, the cancer (e.g., PI3Kα-associated cancer) is selected from breast cancer (including both HER2⁺ and HER2⁻ breast cancer, ER breast cancer, and triple negative breast cancer), uterine cancer (including endometrial cancer), lung cancer (including small cell lung cancer (SCLC) and non-small cell lung cancer (NSCLS, including adenocarcinoma lung cancer and squamous cell lung carcinoma)), esophageal squamous cell carcinoma, ovarian cancer, colorectal cancer, esophagastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocrine neoplasms (pNETs), stomach cancer, esophageal cancer, acute myeloid leukemia, relapsed and refractory multiple myeloma, hepatocellular carcinoma, prostate cancer, Malignant Peripheral Nerve Sheath Tumor (MPNST), glioblastoma, cholangiocarcinoma, and pancreatic cancer.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocrine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments of any of the methods or uses described herein, the cancer (e.g., PI3Kα-associated cancer) is selected from breast cancer, SCLC, NSCLC, endometrial cancer, esophageal squamous cell carcinoma, ovarian cancer, colorectal cancer, esophagastric adenocarcinoma, bladder cancer, head and neck cancer, thyroid cancer, glioma, and cervical cancer.

In some embodiments ofany of the methods or uses described herein, the PI3Kα-associated cancer is breast cancer. In some embodiments ofany of the methods or uses described herein, the PI3Kα-associated cancer is colorectal cancer. In some embodiments ofany of the methods or uses described herein, the PI3Kα-associated cancer is endometrial cancer. In some embodiments of any of the methods or uses described herein, the PI3Kα-associated cancer is lung cancer.

In some embodiments of any of the methods or uses described herein, the PI3Kα-associated cancer is selected from the cancers described in Table 1.

TABLE 1
PI3Kα Protein Amino Acid Substitutions/Insertions/Deletions^{A}

| | Non-Limiting | |
|---|---|---|
| Amino Acid | Exemplary | |
| Position | Mutations | Non-Limiting Exemplary PI3Kα Associated Cancer(s) |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of the Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell Carcinoma |
| | H1047Q, | Uterine Endometrioid Carcinoma |
| | H1047R, H1047Y | Hepatocellular Carcinoma |
| | | Cutaneous Melanoma |
| | | Mucinous Adenocarcinoma of the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic Sarcoma/Malignant Fibrous |
| | | Histiocytoma/High-Grade Spindle Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |

^{A}Unless noted otherwise, the mutations of Table 1 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
† Velho S, Oliveira C, Ferreira A, Ferreira A C, Suriano G, Schwartz S Jr, Duval A, Carneiro F, Machado J C, Hamelin R, Seruca R. The prevalence of PIK3CA mutations in gastric and colon cancer. Eur J Cancer. 2005 July; 41(11): 1649-54. doi: 10.1016/j.ejca.2005.04.022. PMID: 15994075.

Exemplary Sequence of Human Phosphatidylinositol 4,5-bisphosphate 3-kinase isoform alpha (UniProtKB entry P42336) (SEQ ID NO: 1)

MPPRPSSGEL WGIHLMPPRI LVECLLPNGM IVTLECLREA
TLITIKHELF KEARKYPLHQ LLQDESSYIF VSVTQEAERE
EFFDETRRLC DLRLFQPFLK VIEPVGNREE KILNREIGFA
IGMPVCEFDM VKDPEVQDFR RNILNVCKEA VDLRDLNSPH
SRAMYVYPPN VESSPELPKH IYNKLDKGQI IVVIWVIVSP
NNDKQKYTLK INHDCVPEQV IAEAIRKKTR SMLLSSEQLK
LCVLEYQGKY ILKVCGCDEY FLEKYPLSQY KYIRSCIMLG
RMPNLMLMAK ESLYSQLPMD CFTMPSYSRR ISTATPYMNG
ETSTKSLWVI NSALRIKILC ATYVNVNIRD IDKIYVRTGI
YHGGEPLCDN VNTQRVPCSN PRWNEWLNYD IYIPDLPRAA
RLCLSICSVK GRKGAKEEHC PLAWGNINLF DYTDTLVSGK
MALNLWPVPH GLEDLLNPIG VTGSNPNKET PCLELEFDWF
SSVVKFPDMS VIEEHANWSV SREAGFSYSH AGLSNRLARD
NELRENDKEQ LKAISTRDPL SEITEQEKDF LWSHRHYCVT
IPEILPKLLL SVKWNSRDEV AQMYCLVKDW PPIKPEQAME
LLDCNYPDPM VRGFAVRCLE KYLTDDKLSQ YLIQLVQVLK
YEQYLDNLL RFLLKKALTN QRIGHFFFWH LKSEMHNKTV
SQRFGLLLES YCRACGMYLK HLNRQVEAME KLINLTDILK
QEKKDETQKV QMKFLVEQMR RPDFMDALQG FLSPLNPAHQ
LGNLRLEECR IMSSAKRPLW LNWENPDIMS ELLFONNEII
FKNGDDLRQD MLTLQIIRIM ENIWQNQGLD LRMLPYGCLS
IGDCVGLIEV VRNSHTIMQI QCKGGLKGAL QFNSHTLHQW
LKDKNKGEIY DAAIDLFTRS CAGYCVATFI LGIGDRHNSN
IMVKDDGQLF HIDFGHFLDH KKKKFGYKRE RVPFVLTQDF
LIVISKGAQE CTKTREFERF QEMCYKAYLA IRQHANLFIN
LFSMMLGSGM PELQSFDDIA YIRKTLALDK TEQEALEYFM
KQMNDAHHGG WTTKMDWIFH TIKQHALN

Also provided is a method for inhibiting PI3Kα activity in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell. As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of inhibiting cell proliferation, in vitro or in vivo, comprising contacting a cell with an effective amount of a compound of Formula (I), or a pharmaceutically acceptable salt thereof, or a pharmaceutical composition thereof as defined herein.

Further provided herein is a method of increase cell death, in vitro or in vivo, comprising contacting a cell with an effective amount of a compound of Formula (I), or a pharmaceutically acceptable salt thereof, or a pharmaceutical composition thereof as defined herein. Also provided herein is a method of increasing tumor cell death in a subject, comprising administering to the subject an effective compound of Formula (I), or a pharmaceutically acceptable salt thereof, in an amount effective to increase tumor cell death.

In some embodiments, the PI3Kα is human PI3Kα. In some embodiments, the PI3Kα has one or more point mutations in the PIK3CA gene. In some embodiments, the point mutations include a substitution at amino acid position 1047 of a human PI3Kα protein. In some embodiments, the substitution is H1047R.

When employed as pharmaceuticals, the compounds of Formula (I), including pharmaceutically acceptable salts thereof, can be administered in the form of pharmaceutical compositions as described herein.

### EXAMPLES

### Example 1: Compound Preparation

The general methods for the preparation of the compounds of Formula (I) have been described in an illustrative manner and is intended to be description, rather than of limitation. Thus, it will be appreciated that conditions such as choice of solvent, temperature of reaction, volumes, reaction time may vary while still producing the desired compounds. In addition, it will be appreciated that many of the reagents provided in the following examples may be substituted with other suitable reagents. See, e.g., Smith & March, Advanced Organic Chemistry, 7th Ed. (2013). Such changes and modifications, including without limitation, those relating to the chemical structures, substituents, derivatives, intermediates, syntheses, formulations and/or methods of use provided herein, may be made without departing from the spirit and scope thereof.

The starting materials used for the syntheses are either synthesized or obtained from commercial sources, such as, but not limited to, Sigma-Aldrich, Fluka, Acros Organics, Alfa Aesar, Enamine, Strem, VWR Scientific, and the like. Nuclear Magnetic Resonance (NMR) analysis was conducted using a Bruker AVANCE III HD (300 or 400) MHz spectrometer or Bruker AVANCE NEO 400 MHz spectrometer with an appropriate deuterated solvent. LCMS spectra were obtained on a Shimadzu LCMS-2020 with electrospray ionization in positive ion detection mode with 20ADXR pump, SIL-20ACXR autosampler, CTO-20AC column oven, M20A PDA Detector and LCMS 2020 MS detector.

### Compound 25: (S)-2-((1-(2-(2,3-dihydro-1H-inden-2-yl)-6-methyl-1-oxoisoindolin-4-yl)ethyl)amino)benzoic Acid

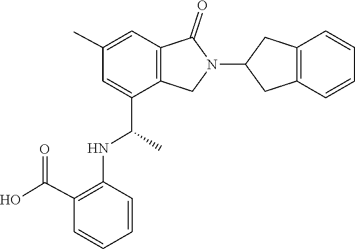 [SMILES: Cc1cc2c(c([C@H](C)Nc3ccccc3C(=O)O)c1)CN(C1Cc3ccccc3C1)C2=O]

### Step 1—Synthesis of methyl 3-bromo-2-(bromomethyl)-5-methylbenzoate

To a mixture of methyl 3-bromo-2,5-dimethylbenzoate (2.1 g, 8.64 mmol, prepared according to the procedure in CN113004145) in CCl₄ (20 mL) was added AIBN (142 mg, 864 umol) and NBS (1.54 g, 8.64 mmol). The mixture was stirred at 80° C. for 16 h. After cooling to room temperature, the reaction was quenched with aq. sat. NaHCO₃ (100 mL). The mixture was stirred for 10 min at room temperature. The aqueous phase was extracted with EtOAc (100 mL×3). The combined organic layers were washed with brine (50 mL×2), dried over anhydrous Na₂SO₄, filtered and concentrated in vacuo. The crude residue was purified by silica gel chromatography (solvent gradient: 0-8% EtOAc in petroleum ether) to give the title compound (1 g, 36%) as colorless oil. ¹H NMR (400 MHz, DMSO-d₆) δ 7.81 (s, 1H), 7.69 (s, 1H), 5.01 (s, 2H), 3.88 (s, 3H), 2.35 (s, 3H).

### Step 2—Synthesis of tert-butyl (5-chloro-2,3-dihydro-1H-inden-2-yl)carbamate

A mixture of 5-chloro-1,3-dihydro-2H-inden-2-one (1 g, 6.0 mmol), NH₄OAc (4.63 mg, 60.0 mmol) and AcOH (36 mg, 0.6 mmol) in MeOH (20 mL) was stirred at room temperature for 1 h. Then the mixture was added NaBH₃CN (755 mg, 12.0 mmol) and stirred at 60° C. for 16 h. After cooling to room temperature, the reaction was added Boc₂O (2.62 g, 12.0 mmol). The mixture was stirred at room temperature for another 2 h. The mixture was quenched with aq. sat. NaHCO₃ (50 mL), extracted with DCM (80 mL×2). The combined organic layers were washed with brine (50 mL), dried over anhydrous Na₂SO₄, filtered and concentrated in vacuo. The crude residue was purified by silica gel chromatography (solvent gradient: 0-10% EtOAc in petroleum ether) to give the title compound (600 mg, 37%) as a brown solid. ¹H NMR (400 MHz, CDCl₃) δ 7.20 (s, 1H), 7.17-7.11 (m, 2H), 4.73 (s, 1H), 4.49-4.45 (m, 1H), 3.33-3.17 (m, 2H), 2.83-2.67 (m, 2H), 1.45 (s, 9H). MS: m/z 212.1 (M−56+H⁺).

### Step 3—Synthesis of 5-chloro-2,3-dihydro-1H-inden-2-amine Hydrochloride

To a solution of tert-butyl (5-chloro-2,3-dihydro-1H-inden-2-yl)carbamate (600 mg, 2.24 mmol) in DCM (10 mL) was added HCl (5 mL, 4 M in dioxane). After the addition, the reaction mixture was stirred at room temperature for 2 h. The mixture was concentrated to give the title compound (440 mg, crude) as a yellow solid that required no further purification. ¹H NMR (400 MHz, DMSO-d₆) δ 8.29 (s, 3H), 7.37 (s, 1H), 7.32-7.27 (m, 1H), 7.27-7.22 (m, 1H), 4.06-3.93 (m, 1H), 3.32-3.20 (m, 2H), 3.01-2.91 (m, 2H). MS: m/z 168.1 (M+H⁺).

### Step 4—Synthesis of 4-bromo-2-(5-chloro-2,3-dihydro-1H-inden-2-yl)-6-methylisoindolin-1-one

A mixture of methyl 3-bromo-2-(bromomethyl)-5-methylbenzoate (400 mg, 1.24 mmol), 5-chloro-2,3-dihydro-1H-inden-2-amine hydrochloride (273 mg, 1.34 mmol) and K₂CO₃ (515 mg, 3.73 mmol) in EtOH (10 mL) was stirred at 60° C. for 16 h. After cooling to room temperature, the reaction was diluted with EtOAc (30 mL) and filtered, the filtrate was concentrated in vacuo. The crude residue was purified by silica gel chromatography (solvent gradient: 0-20% EtOAc in petroleum ether) to give the title compound (160 mg, 34%) as a yellow solid. ¹H NMR (400 MHz, DMSO-d₆) δ 7.65 (s, 1H), 7.53 (s, 1H), 7.33 (s, 1H), 7.31-7.22 (m, 2H), 5.14-5.06 (m, 1H), 4.20 (s, 2H), 3.28-2.97 (m, 4H), 2.40 (s, 3H). MS: m/z 377.9 (M+H⁺).

### Step 5—Synthesis of 2-(5-chloro-2,3-dihydro-1H-inden-2-yl)-4-(1-ethoxyvinyl)-6-methylisoindolin-1-one

A mixture of 4-bromo-2-(5-chloro-2,3-dihydro-1H-inden-2-yl)-6-methylisoindolin-1-one (160 mg, 424 mmol), tributyl(1-ethoxyvinyl)stannane (160 mg, 424 mol) and Pd(dppf)Cl₂ (307 mg, 849 μmol) in dioxane (3 mL) was stirred at 90° C. for 16 h under N₂ atmosphere. After cooling to room temperature, the reaction was quenched with 10% KF solution (20 mL). The mixture was filtered and the filtrate was separated between EtOAc (30 mL) and H₂O (20 mL). The organic layer was washed with brine (20 mL), dried over anhydrous Na₂SO₄, filtered and concentrated in vacuo. The crude residue was purified by silica gel chromatography (solvent gradient: 0-30% EtOAc in petroleum ether) to give the title compound (87 mg, 57%) as an off-white solid. ¹H NMR (400 MHz, CDCl₃) δ 7.62 (s, 1H), 7.49 (s, 1H), 7.29-7.27 (m, 1H), 7.20 (s, 2H), 5.44-5.37 (m, 1H), 4.56 (d, J=2.8 Hz, 1H), 4.22 (d, J=2.8 Hz, 1H), 4.19 (d, J=2.8 Hz, 2H), 3.81-3.74 (m, 2H), 3.40-3.31 (m, 2H), 3.03-2.92 (m, 2H), 2.44 (s, 3H), 1.10 (t, J=7.2 Hz, 3H). MS: m/z 367.9 (M+H⁺).

### Step 6—Synthesis of 4-acetyl-2-(5-chloro-2,3-dihydro-1H-inden-2-yl)-6-methylisoindolin-1-one

To a solution of 2-(5-chloro-2,3-dihydro-1H-inden-2-yl)-4-(1-ethoxyvinyl)-6-methylisoindolin-1-one (87 mg, 236 mol) in THF (2 mL) was added HCl (1 mL, 1 M in water) at room temperature. The reaction mixture was stirred at room temperature for 1 h. The mixture was quenched with saturated NaHCO₃ (10 mL), extracted with EtOAc (30 mL×2). The combined organic layers were washed with brine (20 mL), dried over anhydrous Na₂SO₄ and filtered. The filtrate was concentrated to give the title compound (80 mg, crude) as a yellow solid that required no further purification. ¹H NMR (400 MHz, DMSO-d₆) δ 8.06 (s, 1H), 7.76 (s, 1H), 7.34 (s, 1H), 7.32-7.28 (m, 1H), 7.27-7.22 (m, 1H), 5.15-5.06 (m, 1H), 4.47 (s, 2H), 3.30-3.19 (m, 2H), 3.15-3.04 (m, 2H), 2.59 (s, 3H), 2.49 (s, 3H). MS: m/z 339.9 (M+H⁺).

### Step 7—Synthesis of 4-(1-aminoethyl)-2-(5-chloro-2,3-dihydro-1H-inden-2-yl)-6-methylisoindolin-1-one

A mixture of 4-acetyl-2-(5-chloro-2,3-dihydro-1H-inden-2-yl)-6-methylisoindolin-1-one (80 mg, 235 mol), NH₄OAc (363 mg, 4.71 mmol) and AcOH (1.41 mg, 23.5 μmol) in MeOH (5 mL) was stirred at room temperature for 1 h. Then NaBH₃CN (29.6 mg, 471 μmol) was added to the reaction mixture. After the addition, the reaction mixture was stirred at 60° C. for 16 h. After cooling to room temperature, the reaction mixture was diluted with DCM (30 mL). The mixture was washed with saturated NaHCO₃ solution (20 mL×3), brine (20 mL), dried over anhydrous Na₂SO₄ and filtered. The filtrate was concentrated to give the title compound (80 mg, crude) as a brown solid that required no further purification. MS: m/z 363.1 (M+Na⁺).

Step 8—Synthesis of tert-butyl 2-((1-(2-(5-chloro-2,3-dihydro-1H-inden-2-yl)-6-methyl-1-oxoisoindolin-4-yl)ethyl)amino)benzoate A mixture of 4-(1-aminoethyl)-2-(5-chloro-2,3-dihydro-1H-inden-2-yl)-6-methylisoindolin-1-one (200 mg, 587 μmol), tert-butyl 2-iodobenzoate (357 mg, 1.17 mmol), Cs₂CO₃ (574 mg, 1.76 mmol), Pd₂(dba)₃ (54 mg, 59 μmol) and Xantphos (68 mg, 117 μmol) in dioxane (5 mL), the reaction mixture was stirred at 100° C. for 16 h under N₂ atmosphere. After cooling to room temperature, the reaction was filtered and the filtrate was concentrated. The crude residue was purified by silica gel chromatography (solvent gradient: 0-20% EtOAc in petroleum ether) to give the title compound (120 mg, 40%) as a yellow solid. ¹H NMR (400 MHz, DMSO-d₆) δ 8.08-7.98 (m, 1H), 7.77-7.73 (m, 1H), 7.38 (s, 2H), 7.33-7.26 (m, 2H), 7.23-7.15 (m, 2H), 6.56-6.51 (m, 1H), 6.47-6.42 (m, 1H), 5.12-5.00 (m, 1H), 4.77-4.68 (m, 2H), 4.32-4.24 (m, 1H), 3.21-3.09 (m, 3H), 3.05-2.94 (m, 1H), 2.36 (s, 3H), 1.53 (d, J=6.8 Hz, 3H), 1.51 (s, 9H). MS: m/z 517.1 (M+H⁺).

### Step 9—Synthesis of tert-butyl 2-((1-(2-(2,3-dihydro-1H-inden-2-yl)-6-methyl-1-oxoisoindolin-4-yl)ethyl)amino)benzoate

To a solution of tert-butyl 2-((1-(2-(5-chloro-2,3-dihydro-1H-inden-2-yl)-6-methyl-1-oxoisoindolin-4-yl)ethyl)amino)benzoate (100 mg, 193 μmol) and TEA (75 mg, 741 μmol) in MeOH (5 mL) was added wet Pd(OH)₂ on carbon (50 mg, 20% Pd, 50% wet with water). The reaction was stirred at room temperature for 4 h under H2 atmosphere (15 psi). The reaction was filtered through diatomaceous earth and the filtrate was concentrated to afford the title compound (90 mg, crude) as a yellow solid that required no further purification. MS: m/z 483.2 (M+H⁺).

### Step 10—Synthesis of 2-((1-(2-(2,3-dihydro-1H-inden-2-yl)-6-methyl-1-oxoisoindolin-4-yl)ethyl)amino)benzoic Acid

To a mixture of tert-butyl 2-((1-(2-(2,3-dihydro-1H-inden-2-yl)-6-methyl-1-oxoisoindolin-4-yl)ethyl)amino)benzoate (100 mg, 207 umol) in DCM (3 mL) was added TFA (1 mL, 13.5 mmol). The reaction mixture was stirred at 40° C. for 16 h. After cooling to room temperature, the reaction mixture was concentrated and the residue was purified by reverse phase chromatography (acetonitrile 37%-67%/0.225% formic acid in water) to give the title compound (38 mg, 43%) as a white solid. ¹H NMR (400 MHz, DMSO-d₆) δ 12.76 (s, 1H), 8.26 (s, 1H), 7.81-7.75 (m, 1H), 7.37 (s, 1H), 7.34 (s, 1H), 7.29-7.22 (m, 2H), 7.21-7.15 (m, 3H), 6.56-6.49 (m, 1H), 6.41 (d, J=8.4 Hz, 1H), 5.10-4.99 (m, 1H), 4.76-4.63 (m, 2H), 4.35-4.31 (m, 1H), 3.22-3.17 (m, 2H), 3.17-3.03 (m, 2H), 2.37-2.33 (m, 1H), 2.35 (s, 2H), 1.50 (d, J=6.8 Hz, 3H). MS: m/z 427.2 (M+H⁺).

### Step 11—Synthesis of (R)-2-((1-(2-(2,3-dihydro-1H-inden-2-yl)-6-methyl-1-oxoisoindolin-4-yl)ethyl)amino)benzoic acid and (S)-2-((1-(2-(2,3-dihydro-1H-inden-2-yl)-6-methyl-1-oxoisoindolin-4-yl)ethyl)amino)benzoic Acid

Racemic 2-((1-(2-(2,3-dihydro-1H-inden-2-yl)-6-methyl-1-oxoisoindolin-4-yl)ethyl)amino)benzoic acid (34 mg, 80 umol) was separated by using chiral SFC (DAICEL CHIRALPAK AD(250 mm*30 mm, 10 um); Supercritical CO₂/IPA+0.1% NH₃·H₂O=30/70; 150 mL/min) to afford (R)-2-((1-(2-(2,3-dihydro-1H-inden-2-yl)-6-methyl-1-oxoisoindolin-4-yl)ethyl)amino)benzoic acid (9.6 mg, first peak) and (S)-2-((1-(2-(2,3-dihydro-1H-inden-2-yl)-6-methyl-1-oxoisoindolin-4-yl)ethyl)amino)benzoic acid (8.9 mg, second peak) both as white solid. Absolute configuration was arbitrarily assigned to each enantiomer. Compound 25: ¹H NMR (400 MHz, DMSO-d₆) δ 12.75 (s, 1H), 8.32 (s, 1H), 7.79-7.76 (m, 1H), 7.37 (s, 1H), 7.34 (s, 1H), 7.28-7.21 (m, 2H), 7.21-7.15 (m, 3H), 6.56-6.49 (m, 1H), 6.41 (d, J=8.4 Hz, 1H), 5.10-4.99 (m, 1H), 4.76-4.63 (m, 2H), 4.36-4.31 (m, 1H), 3.23-3.18 (m, 2H), 3.17-3.03 (m, 2H), 2.37-2.33 (m, 1H), 2.35 (s, 2H), 1.50 (d, J=6.4 Hz, 3H). MS: m/z 427.1 (M+H⁺).

### Compound 76: (S)-2-((1-(2-(2,3-dihydro-1H-inden-2-yl)-6-methyl-1-oxoisoindolin-4-yl)ethyl)amino)benzenesulfonamide

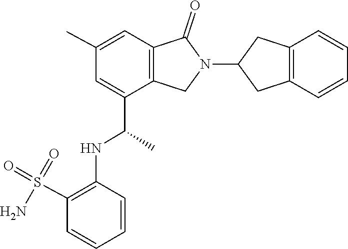 [SMILES: Cc1cc2c(c([C@H](C)Nc3ccccc3S(N)(=O)=O)c1)CN(C1Cc3ccccc3C1)C2=O]

### Step 1—Synthesis of 2-(5-chloro-2,3-dihydro-1H-inden-2-yl)-4-(1-hydroxyethyl)-6-methylisoindolin-1-one

To a solution of 4-acetyl-2-(5-chloro-2,3-dihydro-1H-inden-2-yl)-6-methylisoindolin-1-one (100 mg, 294 μmol) in MeOH (2 mL) was added NaBH₄ (10 mg, 264 mol). The mixture was stirred at room temperature for 1 h. The reaction was quenched with sat. aq. NH₄Cl (5 mL), diluted with water (10 mL), extracted with EtOAc (15 mL×2). The combined organic layers were washed with brine (10 mL), dried over anhydrous Na₂SO₄, filtered and concentrated in vacuo to afford title compound (100 mg, crude) as a yellow solid that required no further purification. ¹H NMR (400 MHz, DMSO-d₆) δ 7.38-7.32 (m, 3H), 7.31-7.27 (m, 1H), 7.26-7.21 (m, 1H), 5.14-5.04 (m, 1H), 4.84-4.70 (m, 1H), 4.38 (s, 2H), 3.28-3.03 (m, 4H), 2.38 (s, 3H), 1.31 (d, J=6.4 Hz, 3H). MS: m/z 342.1 (M+H⁺).

### Step 2—Synthesis of 4-(1-bromoethyl)-2-(5-chloro-2,3-dihydro-1H-inden-2-yl)-6-methylisoindolin-1-one

To a solution of 2-(5-chloro-2,3-dihydro-1H-inden-2-yl)-4-(1-hydroxyethyl)-6-methylisoindolin-1-one (100 mg, 293 mol) in DCM (2 mL) was added PBr₃ (87 mg, 322 mol) at 0° C. The reaction mixture was stirred at 0° C. for 1 h under N₂ atmosphere. The mixture was concentrated in vacuo to afford title compound (500 mg, crude) as a yellow solid that required no further purification. ¹H NMR (400 MHz, DMSO-d₆) δ 7.60 (s, 1H), 7.50-7.44 (m, 1H), 7.35 (s, 1H), 7.17-7.09 (m, 1H), 7.05-6.97 (m, 1H), 5.92 (s, 2H), 5.48 (m, 1H), 5.16-5.05 (m, 1H), 3.30-3.19 (m, 2H), 3.17-3.03 (m, 2H), 2.42 (s, 3H), 2.01 (d, J=6.8 Hz, 3H).

### Step 3—Synthesis of 2-((1-(2-(5-chloro-2,3-dihydro-1H-inden-2-yl)-6-methyl-1-oxoisoindolin-4-yl)ethyl)amino)benzenesulfonamide

To a solution of 4-(1-bromoethyl)-2-(5-chloro-2,3-dihydro-1H-inden-2-yl)-6-methylisoindolin-1-one (110 mg, 272 mol) in dioxane (2 mL) was added 2-aminobenzenesulfonamide (140 mg, 815 μmol). The mixture was stirred at 100° C. for 16 h. After cooling to room temperature, the reaction mixture was added water (20 mL) and extracted with EtOAc (40 mL×2). The combined organic layers were washed with brine (20 mL), dried over anhydrous Na₂SO₄, filtered and concentrated in vacuo. The residue was purified by reverse phase chromatography (acetonitrile 55%-85%/0.225% formic acid in water) to give the title compound (60 mg, 28%) as a white solid. MS: m/z 496.0 (M+H⁺).

### Step 4—Synthesis of 2-((1-(2-(2,3-dihydro-1H-inden-2-yl)-6-methyl-1-oxoisoindolin-4-yl)ethyl)amino)benzenesulfonamide

To a solution of 2-((1-(2-(5-chloro-2,3-dihydro-1H-inden-2-yl)-6-methyl-1-oxoisoindolin-4-yl)ethyl)amino)benzenesulfonamide (60 mg, 120 μmol) and TEA (37 mg, 363.0 μmol) in MeOH (3 mL) was added wet Pd(OH)₂ on carbon (50 mg, 20% Pd, 50% wet with water). The reaction was stirred at room temperature for 2 h under H2 atmosphere (15 psi). The reaction was filtered through diatomaceous earth and the filtrate was concentrated in vacuo. The residue was purified by reverse phase chromatography (acetonitrile 46%-76%/0.225% formic acid in water) to give the title compound (25 mg, 43%) as a white solid. ¹H NMR (400 MHz, DMSO-d₆) δ 7.65-7.61 (m, 1H), 7.56 (s, 2H), 7.47 (s, 1H), 7.38 (s, 1H), 7.30-7.23 (m, 2H), 7.22-7.13 (m, 3H), 6.68-6.60 (m, 1H), 6.46 (d, J=8.4 Hz, 1H), 6.24 (d, J=5.6 Hz, 1H), 5.08-5.02 (m, 1H), 4.80-4.64 (m, 2H), 4.49-4.44 (m, 1H), 3.24-3.10 (m, 4H), 2.35 (s, 3H), 1.52 (d, J=6.8 Hz, 3H). MS: m/z 462.0 (M+H⁺).

### Step 5—Synthesis of (R)-2-((1-(2-(2,3-dihydro-1H-inden-2-yl)-6-methyl-1-oxoisoindolin-4-yl)ethyl)amino)benzenesulfonamide & (S)-2-((1-(2-(2,3-dihydro-1H-inden-2-yl)-6-methyl-1-oxoisoindolin-4-yl)ethyl)amino)benzenesulfonamide

2-((1-(2-(2,3-dihydro-1H-inden-2-yl)-6-methyl-1-oxoisoindolin-4-yl)ethyl)amino)benzene sulfonamide (25 mg, 54 mol) was separated by using chiral SFC (DAICEL CHIRALPAK AD (250 mm*30 mm, 10 um); Supercritical CO₂/EtOH+0.1% NH₃·H₂O=60/40; 80 mL/min) to afford (R)-2-((1-(2-(2,3-dihydro-1H-inden-2-yl)-6-methyl-1-oxoisoindolin-4-yl)ethyl)amino)benzenesulfonamide (4.8 mg, first peak) and (S)-2-((1-(2-(2,3-dihydro-1H-inden-2-yl)-6-methyl-1-oxoisoindolin-4-yl)ethyl)amino)benzenesulfonamide (2.4 mg, second peak) both as yellow solid. Absolute configuration was arbitrarily assigned to each enantiomer. (first peak): ¹H NMR (400 MHz, DMSO-d₆) δ 7.65-7.61 (m, 1H), 7.55 (s, 2H), 7.47 (s, 1H), 7.38 (s, 1H), 7.29-7.22 (m, 2H), 7.22-7.14 (m, 3H), 6.68-6.58 (m, 1H), 6.46 (d, J=8.0 Hz, 1H), 6.23 (d, J=5.2 Hz, 1H), 5.07-5.03 (m, 1H), 4.79-4.66 (m, 2H), 4.49-4.44 (m, 1H), 3.24-3.08 (m, 4H), 2.35 (s, 3H), 1.52 (d, J=6.8 Hz, 3H). MS: m/z 462.0 (M+H⁺). Compound 76 (second peak): ¹H NMR (400 MHz, DMSO-d₆) δ 7.65-7.61 (m, 1H), 7.55 (s, 2H), 7.47 (s, 1H), 7.38 (s, 1H), 7.29-7.23 (m, 2H), 7.21-7.15 (m, 3H), 6.64 (m, 1H), 6.46 (d, J=8.4 Hz, 1H), 6.23 (d, J=5.6 Hz, 1H), 5.07-5.03 (m, 1H), 4.78-4.65 (m, 2H), 4.49-4.44 (m, 1H), 3.23-3.08 (m, 4H), 2.35 (s, 3H), 1.52 (d, J=6.8 Hz, 3H). MS: m/z 462.0 (M+H⁺).

### Compound 61 2-((1-(2-(isochroman-4-yl)-6-methyl-1-oxoisoindolin-4-yl)ethyl)amino)benzoic Acid

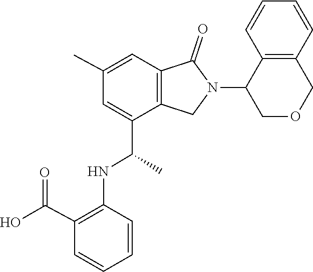 [SMILES: Cc1cc2c(c([C@H](C)Nc3ccccc3C(=O)O)c1)CN(C1COCc3ccccc31)C2=O]

### Step 1—Synthesis of 4-bromo-2-(isochroman-4-yl)-6-methylisoindolin-1-one

A mixture of methyl 3-bromo-2-(bromomethyl)-5-methylbenzoate (200 mg, 621 mol), isochroman-4-amine (111 mg, 745 μmol) and K₂CO₃ (138 mg, 1.86 mmol) in EtOH (3 mL) was stirred at 60° C. for 1 h. After cooling to room temperature, the reaction was diluted with DCM (5 mL) and filtered, the filtrate was concentrated in vacuo. The crude residue was purified by silica gel chromatography (solvent gradient: 0-10% EtOAc in petroleum ether) to give the title compound (208 mg, 79%) as colourless oil. ¹H NMR (400 MHz, DMSO-d₆) δ 7.66 (s, 1H), 7.60 (s, 1H), 7.33-7.28 (m, 1H), 7.25-7.22 (m, 1H), 7.20-7.15 (m, 2H), 5.42-5.36 (m, 1H), 4.95-4.86 (m, 1H), 4.73-4.67 (m, 1H), 4.45-4.36 (m, 1H), 4.21-4.15 (m, 1H), 4.07-4.00 (m, 1H), 3.76-3.70 (m, 1H), 2.41 (s, 3H). MS: m/z 357.9 (M+H⁺).

### Step 2—Synthesis of 4-(1-ethoxyvinyl)-2-(isochroman-4-yl)-6-methylisoindolin-1-one

A mixture of 4-bromo-2-(isochroman-4-yl)-6-methylisoindolin-1-one (208 mg, 581 μmol), tributyl(1-ethoxyvinyl)stannane (530 mg, 1.47 mmol) and Pd(dppf)Cl₂ (43 mg, 58 μmol) in dioxane (2 mL) was stirred at 90° C. for 16 h under N₂ atmosphere. After cooling to room temperature, the reaction was quenched with 10% KF solution (10 mL). The mixture was filtered and the filtrate was separated between EtOAc (40 mL) and H₂O (20 mL). The organic layer was washed with brine (20 mL), dried over anhydrous Na₂SO₄, filtered and concentrated in vacuo. The crude residue was purified by silica gel chromatography (solvent gradient: 0-20% EtOAc in petroleum ether) to give the title compound (133 mg, 66%) as yellow oil. ¹H NMR (400 MHz, DMSO-d₆) δ 7.59 (s, 1H), 7.54 (s, 1H), 7.32-7.27 (m, 1H), 7.24-7.20 (m, 1H), 7.19-7.15 (m, 2H), 5.38-5.33 (m, 1H), 4.91-4.80 (m, 1H), 4.76-4.72 (m, 1H), 4.67-4.65 (m, 1H), 4.62-4.55 (m, 1H), 4.33-4.31 (m, 1H), 4.19-4.12 (m, 1H), 4.06-4.02 (m, 1H), 3.92-3.83 (m, 1H), 3.81-3.76 (m, 2H), 2.41 (s, 3H), 1.17-1.14 (m, 3H) MS: m/z 350.1 (M+H⁺).

### Step 3—Synthesis of 4-acetyl-2-(isochroman-4-yl)-6-methylisoindolin-1-one

To a solution of 4-(1-ethoxyvinyl)-2-(isochroman-4-yl)-6-methylisoindolin-1-one (133 mg, 381 μmol) in THF (2 mL) was added HCl (1 mL, IM in water) at room temperature. The reaction mixture was stirred at room temperature for 1 h. The mixture was quenched with saturated NaHCO₃ (5 mL), extracted with EtOAc (10 mL×2). The combined organic layers were washed with brine (10 mL), dried over anhydrous Na₂SO₄ and filtered. The filtrate was concentrated to give the title compound (122 mg, crude) as a yellow solid that required no further purification. MS: m/z 322.0 (M+H⁺).

### Step 4—Synthesis of 4-(1-aminoethyl)-2-(isochroman-4-yl)-6-methylisoindolin-1-one

A mixture of 4-acetyl-2-(isochroman-4-yl)-6-methylisoindolin-1-one (122 mg, 380 mol), NH₄OAc (439 mg, 5.69 mmol) and AcOH (46 mg, 759 mol) in MeOH (4 mL) was stirred at room temperature for 1 h. Then NaBH₃CN (72 mg, 1.14 μmol) was added to the reaction mixture. After the addition, the reaction mixture was stirred at 60° C. for 16 h. After cooling to room temperature, the reaction mixture was diluted with DCM (30 mL). The mixture was washed with saturated NaHCO₃ solution (20 mL×3), brine (20 mL), dried over anhydrous Na₂SO₄ and filtered. The filtrate was concentrated to give the title compound (112 mg, crude) as yellow oil that required no further purification. MS: m/z 345.0 (M+Na⁺).

### Step 5—Synthesis of tert-butyl 2-((1-(2-(isochroman-4-yl)-6-methyl-1-oxoisoindolin-4-yl)ethyl)amino)benzoate

A mixture of 4-(1-aminoethyl)-2-(isochroman-4-yl)-6-methylisoindolin-1-one (158 mg, 521 μmol), tert-butyl 2-iodobenzoate (322 mg, 347 mol), Cs₂CO₃ (453 mg, 1.39 mmol), Pd₂(dba)₃ (32 mg, 35 μmol) and Xantphos (20 mg, 35 mol) in dioxane (2 mL), the reaction mixture was stirred at 100° C. for 2 h under N₂ atmosphere. After cooling to room temperature, the reaction was filtered and the filtrate was concentrated. The crude residue was purified by silica gel chromatography (solvent gradient: 10-20% EtOAc in petroleum ether) to give the title compound (80 mg, 46%) as yellow oil. ¹H NMR (400 MHz, DMSO-d₆) δ 7.94-7.85 (m, 1H), 7.77-7.64 (m, 1H), 7.45 (s, 1H), 7.37 (d, J=3.2 Hz, 1H), 7.33-7.19 (m, 2H), 7.17-7.06 (m, 3H), 6.55-6.46 (m, 1H), 6.35-6.28 (m, 1H), 5.42-5.34 (m, 1H), 4.90-4.59 (m, 4H), 4.17-4.04 (m, 2H), 3.86-3.75 (m, 1H), 2.40-2.33 (m, 3H), 1.56-1.50 (m, 9H), 1.47-1.41 (m, 3H). MS: m/z 499.3 (M+H⁺).

### Step 6—Synthesis of 2-((1-(2-(isochroman-4-yl)-6-methyl-1-oxoisoindolin-4-yl)ethyl)amino)benzoic Acid

To a mixture of tert-butyl 2-((1-(2-(2,3-dihydro-1H-inden-2-yl)-6-methyl-1-oxoisoindolin-4-yl)ethyl)amino)benzoate (25 mg, 50 umol) in DCM (0.9 mL) was added TFA (0.3 mL, 3.37 mmol). The reaction mixture was stirred at room temperature for 1 h. After cooling to room temperature, the reaction mixture was concentrated and the residue was purified by reverse phase chromatography (acetonitrile 41%-71%/0.225% formic acid in water) to give the title compound (1.85 mg, 8%) as a white solid. ¹H NMR (400 MHz, DMSO-d₆) δ 12.58 (s, 1H), 8.23-8.16 (m, 1H), 7.82-7.61 (m, 1H), 7.84-7.62 (m, 1H), 7.36 (d, J=4.4 Hz, 1H), 7.33-6.92 (m, 5H), 6.54-6.40 (m, 1H), 6.30 (d, J=8.4 Hz, 1H), 5.37-5.34 (m, 1H), 4.91-4.80 (m, 1H), 4.73-4.62 (m, 2.5H), 4.42-4.38 (m, 0.5H), 4.15-3.97 (m, 2.5H), 3.71-3.69 (m, 0.5H), 2.39-2.35 (m, 3H), 1.42 (d, J=5.6 Hz, 3H). MS: m/z 443.1 (M+H⁺).

### Compound 117: 2-((1-(2-((1R,3s,5S)-8-acetyl-8-azabicyclo[3.2.1]octan-3-yl)-6-methyl-1-oxoisoindolin-4-yl)ethyl)amino)benzoic Acid

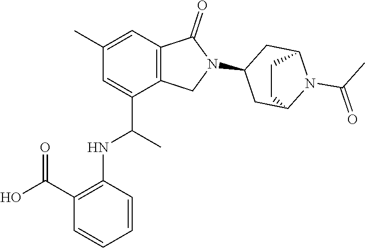 [SMILES: CC(=O)N1[C@@H]2CC[C@H]1C[C@H](N1Cc3c(cc(C)cc3C(C)Nc3ccccc3C(=O)O)C1=O)C2]

### Step 1—Synthesis of (1R,3r,5S)-tert-butyl 3-((2-bromo-6-chloro-4-methylbenzyl)amino)-8-azabicyclo[3.2.1]octane-8-carboxylate & tert-butyl (1R,3s,5S)-3-((2-bromo-6-chloro-4-methylbenzyl)amino)-8-azabicyclo[3.2.1]octane-8-carboxylate

To a solution of 2-bromo-6-chloro-4-methylbenzaldehyde (4.3 g, 18.42 mmol) and tert-butyl 3-amino-8-azabicyclo[3.2.1]octane-8-carboxylate (4.17 g, 18.42 mmol) in MeOH (120 mL) was added AcOH (2.21 g, 36.83 mmol) and NaBH₃CN (1.74 g, 27.63 mmol). The reaction mixture was stirred at room temperature for 16 h. The mixture was quenched with sat. aq. NaHCO₃ (100 mL), extracted with EtOAc (100 mL×2). The combined organic layers were washed with brine (100 mL), dried over anhydrous Na₂SO₄, filtered and concentrated in vacuo. The crude residue was purified by silica gel chromatography (solvent gradient: 0-20% EtOAc in petroleum ether) to give the first peak (2.1 g, 26%) and second peak (4.8 g, 59%) both as white solid, which were further confirmed by NOESY. First peak: ¹H NMR (400 MHz, DMSO-d₆) δ 7.45 (s, 1H), 7.32 (s, 1H), 4.66-4.64 (m, 1H), 3.99-3.93 (m, 2H), 3.86 (s, 2H), 2.90-2.87 (m, 1H), 2.27 (s, 3H), 2.13-2.08 (m, 2H), 1.89-1.79 (m, 2H), 1.78-1.72 (s, 2H), 1.71-1.59 (m, 2H), 1.38 (s, 9H). MS: m/z 443.2 (M+H⁺). Second peak: ¹H NMR (400 MHz, DMSO-d₆) δ 7.44 (s, 1H), 7.31 (s, 1H), 4.07-4.02 (m, 2H), 3.87 (s, 2H), 3.03-2.89 (m, 1H), 2.27 (s, 3H), 1.88-1.73 (m, 4H), 1.65-1.53 (m, 2H), 1.39 (s, 9H), 1.36-1.25 (m, 2H). MS: m/z 443.2 (M+H⁺).

### Step 2—Synthesis of tert-butyl (1R,3s,5S)-3-(4-chloro-6-methyl-1-oxoisoindolin-2-yl)-8-azabicyclo[3.2.1]octane-8-carboxylate

A mixture of tert-butyl (1R,3s,5S)-3-((2-bromo-6-chloro-4-methylbenzyl)amino)-8-azabicyclo[3.2.1]octane-8-carboxylate (4.8 g, 10.82 mmol), Pd(dppf)Cl₂ (2.37 g, 3.24 mmol) and DIPEA (4.19 g, 32.45 mmol, 5.65 mL) in DMF (100 mL) was stirred at 80° C. for 16 h under CO atmosphere (50 psi). After cooling to room temperature, the reaction was quenched with water (200 mL), extracted with EtOAc (200 mL×2). The combined organic layers were washed with brine (200 mL×3), dried over anhydrous Na₂SO₄, filtered and concentrated in vacuo. The crude residue was purified by silica gel chromatography (solvent gradient: 0-50% EtOAc in petroleum ether) to give the title compound (3.1 g, 73%) as a yellow solid. ¹H NMR (400 MHz, DMSO-d₆) δ 7.50 (s, 1H), 7.47 (s, 1H), 4.61-4.52 (m, 1H), 4.35 (s, 2H), 4.17 (s, 2H), 2.40 (s, 3H), 1.99-1.82 (m, 4H), 1.78-1.74 (m, 2H), 1.70-1.62 (m, 2H), 1.45 (s, 9H). MS: m/z 335.2 (M−56+H⁺).

### Step 3—Synthesis of tert-butyl (1R,3s,5S)-3-(4-acetyl-6-methyl-1-oxoisoindolin-2-yl)-8-azabicyclo[3.2.1]octane-8-carboxylate

To a solution of tert-butyl (1R,3s,5S)-3-(4-chloro-6-methyl-1-oxoisoindolin-2-yl)-8-azabicyclo[3.2.1]octane-8-carboxylate (3.1 g, 7.93 mmol) in toluene (50 mL) was added tributyl(1-ethoxyvinyl)stannane (4.3 g, 11.9 mmol), SPhos Pd G3 (619 mg, 793 μmol) and CsF (2.41 g, 15.86 mmol). The reaction was stirred at 100° C. for 16 h under N₂ atmosphere. After cooling to room temperature, the reaction mixture was added 1 M HCl (10 mL) and stirred at room temperature for 0.5 h. The reaction mixture was added 50 mL 10% KF solution, stirred at room temperature over 2 h. The mixture was extracted with EtOAc (200 mL×2). The combined organic phase was washed with brine (200 mL), dried over anhydrous Na₂SO₄, filtered and concentrated in vacuo. The crude residue was purified by silica gel chromatography (solvent gradient: 0-30% EtOAc in petroleum ether) to give the title compound (1.4 g, 60%) as a yellow solid. ¹H NMR (400 MHz, DMSO-d₆) δ 8.07 (s, 1H), 7.74 (s, 1H), 4.62-4.56 (m, 1H), 4.55 (s, 2H), 4.15 (s, 2H), 2.62 (s, 3H), 2.48 (s, 3H), 2.00-1.87 (m, 4H), 1.79-1.75 (m, 2H), 1.70-1.62 (m, 2H), 1.46 (s, 9H). MS: m/z 343.2 (M−56+H⁺).

### Step 4—Synthesis of tert-butyl (1R,3s,5S)-3-(4-(1-aminoethyl)-6-methyl-1-oxoisoindolin-2-yl)-8-azabicyclo[3.2.1]octane-8-carboxylate

To a solution of tert-butyl (1R,3s,5S)-3-(4-acetyl-6-methyl-1-oxoisoindolin-2-yl)-8-azabicyclo[3.2.1]octane-8-carboxylate (1.4 g, 3.51 mmol), NH₄OAc (4.06 g, 52.70 mmol) and AcOH (211 mg, 3.51 mmol) in MeOH (35 mL) was degassed and stirred at room temperature for 1 h, then added NaBH₃CN (662 mg, 10.54 mmol), the mixture was stirred at 60° C. for 16 h. After cooling to room temperature, the reaction mixture was diluted with DCM (150 mL). The mixture was washed with saturated NaHCO₃ solution (100 mL×3), brine (100 mL), dried over anhydrous Na₂SO₄ and filtered. The filtrate was concentrated to give the title compound (1.38 g, crude) as yellow oil that required no further purification. MS: m/z 344.2 (M−56+H⁺).

### Step 5—Synthesis of tert-butyl (1R,3s,5S)-3-(4-(1-((2-(methoxycarbonyl)phenyl)amino)ethyl)-6-methyl-1-oxoisoindolin-2-yl)-8-azabicyclo[3.2.1]octane-8-carboxylate

To a solution of tert-butyl (1R,3s,5S)-3-(4-(1-aminoethyl)-6-methyl-1-oxoisoindolin-2-yl)-8-azabicyclo[3.2.1]octane-8-carboxylate (1.38 g, 3.45 mmol), methyl 2-iodobenzoate (1.36 g, 5.18 mmol), Cs₂CO₃ (3.38 g, 10.36 mmol) and Xantphos (200 mg, 345 mol) in dioxane (30 mL) was added Pd₂(dba)₃ (158 mg, 173 μmol). The reaction was stirred at 100° C. for 2 h under N₂ atmosphere. After cooling to room temperature, the reaction was quenched with water (100 mL), extracted with EtOAc (100 mL×2). The combined organic layers were washed with water (50 mL×3) and brine (50 mL), dried over anhydrous Na₂SO₄, filtered and concentrated in vacuo. The crude residue was purified by silica gel chromatography (solvent gradient: 0-30% EtOAc in petroleum ether) to give the title compound (980 mg, 53%) as a yellow solid. MS: m/z 478.3 (M−56+H⁺).

### Step 6—Synthesis of methyl 2-((1-(2-((1R,3s,5S)-8-azabicyclo[3.2.1]octan-3-yl)-6-methyl-1-oxoisoindolin-4-yl)ethyl)amino)benzoate Hydrochloride

To a solution of tert-butyl (1R,3s,5S)-3-(4-(1-((2-(methoxycarbonyl)phenyl)amino)ethyl)-6-methyl-1-oxoisoindolin-2-yl)-8-azabicyclo[3.2.1]octane-8-carboxylate (980 mg, 1.84 mmol) in DCM (3 mL) was added HCl (5 mL, 4 M in dioxane). The reaction was stirred at room temperature for 1 h under N₂ atmosphere. The reaction mixture was concentrated to give the title compound (860 mg, crude) as a yellow solid that required no further purification. MS: m/z 434.3 (M+H⁺).

### Step 7—Synthesis of methyl 2-((1-(2-((1R,3s,5S)-8-acetyl-8-azabicyclo[3.2.1]octan-3-yl)-6-methyl-1-oxoisoindolin-4-yl)ethyl)amino)benzoate

To a solution of methyl 2-((1-(2-((1R,3s,5S)-8-azabicyclo[3.2.1]octan-3-yl)-6-methyl-1-oxoisoindolin-4-yl)ethyl)amino)benzoate hydrochloride (100 mg, 213 μmol) and TEA (65 mg, 638 mol) in DCM (4 mL) was added Ac₂O (26 mg, 255 μmol). The reaction was stirred at room temperature for 1 h. The reaction was quenched with NaHCO₃ (30 mL), extracted with DCM (30 mL×2). The combined organic layers were washed with brine (30 mL), dried over anhydrous Na₂SO₄, filtered and concentrated in vacuo to give the title compound (100 mg, 99%) as a yellow solid that required no further purification. MS: m/z 476.3 (M+H⁺).

### Step 8—Synthesis of 2-((1-(2-((1R,3s,5S)-8-acetyl-8-azabicyclo[3.2.1]octan-3-yl)-6-methyl-1-oxoisoindolin-4-yl)ethyl)amino)benzoic Acid

To a solution of methyl 2-((1-(2-((1R,3s,5S)-8-acetyl-8-azabicyclo[3.2.1]octan-3-yl)-6-methyl-1-oxoisoindolin-4-yl)ethyl)amino)benzoate (100 mg, 212 μmol) and KOH (119 mg, 2.12 mmol) in EtOH (5 mL) and H₂O (0.1 mL) was stirred at 100° C. for 1 h. After cooling to room temperature, the mixture was adjusted to pH 4 with formic acid, the reaction mixture was concentrated and the residue was purified by reverse phase chromatography (acetonitrile 30%-60%/0.225% formic acid in water) to give the title compound (60 mg, 61%) as a white solid. ¹H NMR (400 MHz, DMSO-d₆) δ 12.81 (s, 1H), 8.32 (s, 1H), 7.79 (d, J=8.0 Hz, 1H), 7.35 (s, 1H), 7.31 (d, J=4.0 Hz, 1H), 7.22-7.14 (m, 1H), 6.54-6.51 (m, 1H), 6.44-6.37 (m, 1H), 4.80-4.73 (m, 1H), 4.67-4.59 (m, 2H), 4.57-4.55 (m, 1H), 4.35-4.27 (m, 2H), 2.32 (s, 3H), 2.03 (s, 3H), 2.01-1.82 (m, 4H), 1.82-1.60 (m, 4H), 1.54-1.50 (m, 3H). MS: m/z 462.3 (M+H⁺).

### Compound 126: 2-((1-(6-methyl-1-oxo-2-(5,6,7,8-tetrahydroquinolin-7-yl)isoindolin-4-yl)ethyl)amino)benzoic Acid

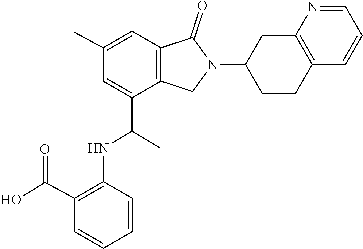 [SMILES: Cc1cc2c(c(C(C)Nc3ccccc3C(=O)O)c1)CN(C1CCc3cccnc3C1)C2=O]

### Step 1—Synthesis of 4-bromo-6-methyl-2-(5,6,7,8-tetrahydroquinolin-7-yl)isoindolin-1-one

A mixture of 5,6,7,8-tetrahydroquinolin-7-amine (276 mg, 1.86 mmol) (prepared according to the procedure in Tetrahedron. Lett., 1991, 32, 6789), methyl 3-bromo-2-(bromomethyl)-5-methylbenzoate (300 mg, 932 μmol) and K₂CO₃ (386 mg, 2.80 mmol) in EtOH (5 mL) was stirred at 60° C. for 16 h. After cooling to room temperature, the reaction was diluted with EtOAc (15 mL) and filtered, the filtrate was concentrated in vacuo. The crude residue was purified by silica gel chromatography (solvent gradient: 0-13% (EtOAc/EtOH=3/1) in petroleum ether) to give the title compound (183 mg, 49%) as a white solid. ¹H NMR (400 MHz, DMSO-d₆) δ 8.34 (d, J=3.6 Hz, 1H), 7.68 (s, 1H), 7.59-7.50 (m, 2H), 7.21-7.13 (m, 1H), 4.56-4.46 (m, 2H), 4.43-4.36 (m, 1H), 3.25-3.15 (m, 1H), 3.06-2.98 (m, 1H), 2.98-2.89 (m, 2H), 2.42 (s, 3H), 2.11-2.02 (m, 2H). MS: m/z 357.1 (M+H⁺).

### Step 2—Synthesis of 4-acetyl-6-methyl-2-(5,6,7,8-tetrahydroquinolin-7-yl)isoindolin-1-one

A mixture of 4-bromo-6-methyl-2-(5,6,7,8-tetrahydroquinolin-7-yl)isoindolin-1-one (183 mg, 512 μmol), tributyl(1-ethoxyvinyl)stannane (450 mg, 1.25 mmol) and Pd(dppf)Cl₂ (19 mg, 26 μmol) in dioxane (2 mL) was stirred at 90° C. for 16 h under N₂ atmosphere. After cooling to room temperature, the reaction mixture was added 1 M HCl (3 mL) and stirred at room temperature for 0.5 h. The reaction mixture was added 15 mL 10% KF solution, stirred at room temperature over 2 h. The mixture was extracted with EtOAc (30 mL×2). The combined organic layers were washed with brine (30 mL), dried over anhydrous Na₂SO₄, filtered and concentrated in vacuo. The crude residue was purified by silica gel chromatography (solvent gradient: 0-13% (EtOAc/EtOH=3/1) in petroleum ether) to give the title compound (65 mg, 58%) as a white solid. ¹H NMR (400 MHz, DMSO-d₆) δ 8.37-8.31 (m, 1H), 8.10 (s, 1H), 7.79 (s, 1H), 7.55 (d, J=7.6 Hz, 1H), 7.21-7.15 (m, 1H), 4.87-4.63 (m, 2H), 4.57-4.44 (m, 1H), 3.25-3.14 (m, 1H), 3.09-2.98 (m, 1H), 2.98-2.87 (m, 2H), 2.69-2.53 (m, 3H), 2.49-2.38 (m, 3H), 2.12-2.02 (m, 2H). MS: m/z 321.2 (M+H⁺).

### Step 3—Synthesis of 4-(1-aminoethyl)-6-methyl-2-(5,6,7,8-tetrahydroquinolin-7-yl)isoindolin-1-one

To a solution of 4-acetyl-6-methyl-2-(5,6,7,8-tetrahydroquinolin-7-yl)isoindolin-1-one (62 mg, 194 μmol), NH₄OAc (149 mg, 1.94 mmol) and AcOH (3 mg, 52 μmol) in MeOH (2 mL) was added NaBH₃CN (36 mg, 581 μmol). The reaction was heated to 60° C. for 16 h under N₂ atmosphere. After cooling to room temperature, the reaction mixture was diluted with DCM (40 mL), washed with saturated NaHCO₃ solution (20 mL×3) and brine (30 mL), dried over anhydrous Na₂SO₄ and filtered. The filtrate was concentrated to give the title compound (62 mg, crude) as a brown solid that required no further purification. MS: m/z 322.3 (M+H⁺).

### Step 4—Synthesis of tert-butyl 2-((1-(6-methyl-1-oxo-2-(5,6,7,8-tetrahydroquinolin-7-yl)isoindolin-4-yl)ethyl)amino)benzoate

A mixture of 4-(1-aminoethyl)-6-methyl-2-(5,6,7,8-tetrahydroquinolin-7-yl)isoindolin-1-one (62 mg, 193 μmol), tert-butyl 2-iodobenzoate (117 mg, 386 mol), Xantphos (22 mg, 39 μmol), Pd₂(dba)₃ (18 mg, 19 μmol) and Cs₂CO₃ (189 mg, 579 mol) in dioxane (1 mL) was heated to 100° C. for 2 h under N₂ atmosphere. After cooling to room temperature, the reaction was quenched with water (10 mL) and extracted with EtOAc (15 mL×2). The combined organic layers were washed with brine (15 mL), dried over anhydrous Na₂SO₄, filtered and concentrated in vacuo. The residue was purified by flash silica gel chromatography (0-18% (EtOAc/EtOH=3/1) in petroleum ether) to give the title compound (32 mg, 33%) as a yellow solid. ¹H NMR (400 MHz, DMSO-d₆) δ 8.35-8.33 (m, 1H), 8.12-8.04 (m, 1H), 7.76 (d, J=7.6 Hz, 1H), 7.54 (d, J=7.6 Hz, 1H), 7.39 (s, 2H), 7.24-7.15 (m, 2H), 6.61-6.41 (m, 2H), 4.92-4.68 (m, 2H), 4.56-4.44 (m, 1H), 4.41-4.24 (m, 1H), 3.10-2.98 (m, 2H), 2.97-2.91 (m, 2H), 2.37 (s, 3H), 1.57 (d, J=6.8 Hz, 3H), 1.52-1.49 (m, 9H), 1.25-1.21 (m, 2H). MS: m/z 498.0 (M+H⁺).

### Step 5—Synthesis of 2-((1-(6-methyl-1-oxo-2-(5,6,7,8-tetrahydroquinolin-7-yl)isoindolin-4-yl)ethyl)amino)benzoic Acid

To a solution of tert-butyl 2-((1-(6-methyl-1-oxo-2-(5,6,7,8-tetrahydroquinolin-7-yl)isoindolin-4-yl)ethyl)amino)benzoate (32 mg, 64 μmol) in DCM (1.5 mL) was added TFA (0.5 mL, 6.8 mmol). The mixture was stirred at 40° C. for 16 h. After cooling to room temperature, the reaction mixture was concentrated in vacuo. The resulting residue was purified by reverse phase chromatography (acetonitrile 20%-50%/0.225% formic acid in water) to give the title compound (10 mg, 35%) as a white solid. ¹H NMR (400 MHz, DMSO-d₆) δ 8.66-8.40 (m, 1H), 8.37-8.31 (m, 1H), 7.79 (d, J=8.0 Hz, 1H), 7.55 (d, J=7.6 Hz, 1H), 7.40-7.35 (m, 2H), 7.22-7.11 (m, 2H), 6.52-6.49 (m, 1H), 6.47-6.40 (m, 1H), 4.91-4.64 (m, 2H), 4.54-4.34 (m, 2H), 3.11-3.03 (m, 1H), 3.03-2.97 (m, 1H), 2.97-2.89 (m, 2H), 2.36 (s, 3H), 2.17-2.03 (m, 1H), 2.00-1.94 (m, 1H), 1.54 (d, J=6.4 Hz, 3H). MS: m/z 442.0 (M+H⁺).

### Compound 150: 2-((1-(6-methyl-1-oxo-2-(5-sulfamoyl-2,3-dihydro-1H-inden-2-yl)isoindolin-4-yl)ethyl)amino)benzoic Acid

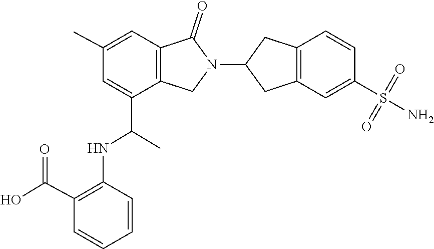 [SMILES: Cc1cc2c(c(C(C)Nc3ccccc3C(=O)O)c1)CN(C1Cc3ccc(S(N)(=O)=O)cc3C1)C2=O]

### Step 1—Synthesis of 2-(4-bromo-6-methyl-1-oxoisoindolin-2-yl)-2,3-dihydro-1H-indene-5-sulfonamide

A solution of methyl 3-bromo-2-(bromomethyl)-5-methylbenzoate (100 mg, 0.31 mmol), 2-amino-2,3-dihydro-1H-indene-5-sulfonamide (77 mg, 0.31 mmol) (prepared according to the procedure in Bioorg. Med. Chem. Lett., 2004, 14, 5781) and K₂CO₃ (128 mg, 0.93 mmol) in EtOH (10 ml) was stirred at 60° C. for 1 h. After cooling to room temperature, the reaction was diluted with EtOAc (30 mL) and filtered, the filtrate was concentrated in vacuo. The residue was concentrated to give the title compound (100 mg, crude) as a yellow solid that required no further purification. MS: m/z 442.7 (M+Na⁺).

### Step 2—Synthesis of 2-(4-acetyl-6-methyl-1-oxoisoindolin-2-yl)-2,3-dihydro-1H-indene-5-sulfonamide

A mixture of 2-(4-bromo-6-methyl-1-oxoisoindolin-2-yl)-2,3-dihydro-1H-indene-5-sulfonamide (620 mg, 1.47 mmol), tributyl(1-ethoxyvinyl)stannane (1.09 g, 3.02 mmol) and Pd(dppf)Cl₂ (107 mg, 0.15 mmol) in dioxane (10 mL) was degassed and purged with N₂ for 3 times. The reaction mixture was stirred at 90° C. for 16 h under N₂ atmosphere. After cooling to room temperature, HCl (4 mL, 1 M) was added. The mixture was stirred at room temperature for 0.5 h. The reaction mixture was added 20 mL 10% KF aqueous solution, stirred at room temperature for 2 h. The mixture was extracted with EtOAc (20 mL×3). The combined organic layers were washed with brine (20 mL), dried over anhydrous Na₂SO₄, filtered and concentrated. The crude residue was purified by silica gel chromatography (solvent gradient: 0-20% EtOAc in petroleum ether) to give the title compound (330 mg, 76%) as a white solid. ¹H NMR (400 MHz, DMSO-d₆) δ 8.08 (s, 1H), 7.77 (s, 1H), 7.72 (s, 1H), 7.68 (d, J=8.0 Hz, 1H), 7.46 (d, J=7.6 Hz, 1H), 7.31 (s, 2H), 5.19-5.11 (m, 1H), 4.52 (s, 2H), 3.26-3.14 (m, 4H), 2.59 (s, 3H), 2.48 (s, 3H) MS: m/z 385.1 (M+H⁺).

### Step 3—Synthesis of tert-butyl N-(2-(4-acetyl-6-methyl-1-oxo-isoindolin-2-yl)indan-5-yl)sulfonyl-N-tert-butoxycarbonyl-carbamate

To a mixture of Boc₂O (450 mg, 2.06 mmol) and 2-(4-acetyl-6-methyl-1-oxo-isoindolin-2-yl)indane-5-sulfonamide (330 mg, 858 μmol) in DCM (5 mL) was added DMAP (11 mg, 86 mol). After the addition, the reaction mixture was stirred at room temperature for 1 h. The mixture was diluted with EtOAc (30 mL), washed with 1 M HCl (20 mL) and brine (20 mL), dried over Na₂SO₄ and filtered. The filtrate was concentrated in vacuo. The crude residue was purified by silica gel chromatography (solvent gradient: 0-50% EtOAc in petroleum ether) to give the title compound (150 mg, 28%) as a white solid. ¹H NMR (400 MHz, DMSO-d₆) δ 8.07 (s, 1H), 7.88 (s, 1H), 7.84 (d, J=8.0 Hz, 1H), 7.77 (s, 1H), 7.52 (d, J=7.6 Hz, 1H), 5.20-5.13 (m, 1H), 4.49 (s, 2H), 3.39-3.35 (m, 2H), 3.26-3.18 (m, 2H), 2.58 (s, 3H), 1.47 (s, 9H), 1.42 (s, 9H). MS: m/z 429.1 (M−100−56+H⁺).

### Step 4—Synthesis of tert-butyl N-(2-[4-(1-aminoethyl)-6-methyl-1-oxo-isoindolin-2-yl]indan-5-yl)sulfonyl-N-tert-butoxycarbonyl-carbamate

To a solution of tert-butyl N-(2-(4-acetyl-6-methyl-1-oxo-isoindolin-2-yl)indan-5-yl)sulfonyl-N-tert-butoxycarbonyl-carbamate (150 mg, 0.25 mmol), NH₄OAc (296 mg, 3.85 mmol) in MeOH (3 mL) was added NaBH₃CN (48 mg, 0.77 mmol). The reaction was heated to 60° C. for 16 h. After cooling to room temperature, the mixture was diluted with EtOAc (50 mL), washed with sat. aq. NaHCO₃ (20 mL×2) and brine (20 mL). The organic layer was dried over Na₂SO₄, filtered and concentrated in vacuo. The residue was concentrated to give the title compound (110 mg, crude) as a yellow oil that required no further purification. MS: m/z 486.2 (M−100+H⁺).

### Step 5—Synthesis of tert-butyl 2-(1-(2-(5-(bis(tert-butoxycarbonyl)sulfamoyl)indan-2-yl)-6-methyl-1-oxo-isoindolin-4-yl)ethylamino)benzoate

A mixture of tert-butyl N-(2-(4-(1-aminoethyl)-6-methyl-1-oxo-isoindolin-2-yl)indan-5-yl)sulfonyl-N-tert-butoxycarbonyl-carbamate (100 mg, 0.17 mmol), tert-butyl 2-bromobenzoate (78 mg, 0.26 mmol), Cs₂CO₃ (167 mg, 0.51 mmol), Pd₂(dba)₃ (15 mg, 17 μmol) and Xantphos (19 mg, 34 mol) in dioxane (0.5 mL) was stirred at 100° C. for 16 h under N₂ atmosphere. After cooling to room temperature, the reaction was diluted with EtOAc (30 mL) and filtered, the filtrate was concentrated in vacuo. The crude residue was purified by silica gel chromatography (solvent gradient: 0-25% EtOAc in petroleum ether) to give the title compound (50 mg, 38%) as a white solid. ¹H NMR (400 MHz, DMSO-d₆) δ 8.09-8.03 (m, 1H), 7.89-7.78 (m, 2H), 7.76-7.73 (m, 1H), 7.51-7.44 (m, 1H), 7.38 (s, 2H), 7.21-7.18 (m, 1H), 6.55-6.53 (m 1H), 6.45 (d, J=8.4 Hz, 1H), 5.14-5.10 (m, 1H), 4.81-4.68 (m, 2H), 4.33-4.27 (m, 1H), 3.29-3.26 (m, 2H), 3.24-3.07 (m, 2H), 2.36 (s, 3H), 1.55-1.49 (m, 12H), 1.47 (s, 9H), 1.40 (s, 9H). MS: m/z 684.3 (M−100+Na⁺).

### Step 6—Synthesis of 2-((1-(6-methyl-1-oxo-2-(5-sulfamoyl-2,3-dihydro-1H-inden-2-yl)isoindolin-4-yl)ethyl)amino)benzoic Acid

To a mixture of tert-butyl 2-(1-(2-(5-(bis(tert-butoxycarbonyl)sulfamoyl)indan-2-yl)-6-methyl-1-oxo-isoindolin-4-yl)ethylamino)benzoate (25 mg, 32 umol) in DCM (1 mL) was added TFA (0.5 mL, 6.9 mmol). The reaction mixture was stirred at room temperature for 16 h. The mixture was concentrated and the residue was purified by reverse phase chromatography (acetonitrile 39%-69%/0.225% formic acid in water) to give the title compound (2 mg, 16%) as a white solid. ¹H NMR (400 MHz, DMSO-d₆) δ 12.76 (s, 1H), 8.34-8.24 (m, 1H), 7.82-7.77 (m, 1H), 7.73-7.69 (m, 1H), 7.69-7.64 (m, 1H), 7.46-7.42 (m, 1H), 7.38 (s, 1H), 7.34 (s, 1H), 7.29 (s, 2H), 7.23-7.16 (m, 1H), 6.54-6.52 (m, 1H), 6.43 (d, J=8.4 Hz, 1H), 5.13-5.07 (m, 1H), 4.79-4.67 (m, 2H), 4.46-4.34 (m, 1H), 3.31-3.26 (m, 2H), 3.25-3.14 (m, 2H), 2.35 (s, 3H), 1.51 (d, J=6.8 Hz, 3H). MS: m/z 528.0 (M+Na⁺).

### Compound 45: 2-((1-(2-(chroman-3-yl)-5,6-dimethyl-1-oxoisoindolin-4-yl)ethyl)amino)benzoic Acid

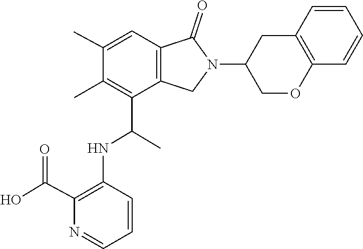 [SMILES: Cc1cc2c(c(C(C)Nc3cccnc3C(=O)O)c1C)CN(C1COc3ccccc3C1)C2=O]

### Step 1—Synthesis of tert-butyl 2-((1-(2-(chroman-3-yl)-5,6-dimethyl-1-oxoisoindolin-4-yl)ethyl)amino)benzoate

To a solution of 4-(1-bromoethyl)-2-(chroman-3-yl)-5,6-dimethylisoindoline-1-one (80 mg, 200 μmol) in dioxane (1 mL) was added tert-butyl 2-aminobenzoate (77 mg, 400 μmol). The mixture was stirred at 100° C. for 16 h. After cooling to room temperature, the reaction mixture was concentrated in vacuo. The residue was purified by prep_TLC (petroleum ether/EtOAc=2/1) to give the title compound (30 mg, 25%) as a white solid. MS: m/z 535.1 (M+Na⁺).

### Step 2—Synthesis of 2-((1-(2-(chroman-3-yl)-5,6-dimethyl-1-oxoisoindolin-4-yl)ethyl)amino)benzoic Acid

To a mixture of tert-butyl 2-((1-(2-(chroman-3-yl)-5,6-dimethyl-1-oxoisoindolin-4-yl)ethyl)amino)benzoate (30 mg, 59 mol) in DCM (3 mL) was added TFA (1 mL, 13.46 mmol). The reaction mixture was stirred at room temperature for 16 h. The mixture was concentrated and the residue was purified by reverse phase chromatography (acetonitrile 47%-77%/0.225% formic acid in water) to give the title compound (4.92 mg, 18%) as a white solid. ¹H NMR (400 MHz, DMSO-d₆) δ 8.28 (s, 1H), 7.87-7.74 (m, 1H), 7.45-7.36 (m, 1H), 7.18-7.03 (m, 3H), 6.91-6.74 (m, 2H), 6.61-6.44 (m, 1H), 6.26-6.10 (m, 1H), 5.04-4.89 (m, 1H), 4.82-4.64 (m, 1H), 4.50-4.36 (m, 1H), 4.27-3.93 (m, 3H), 3.28-2.80 (m, 2H), 2.43 (s, 3H), 2.37 (s, 3H), 1.53-1.46 (m, 3H). MS: m/z 479.1 (M+Na⁺).

### Compound 128: 2-((1-(2-(2-acetyl-2-azaspiro[3.5]nonan-7-yl)-6-methyl-1-oxoisoindolin-4-yl)ethyl)amino)benzoic Acid

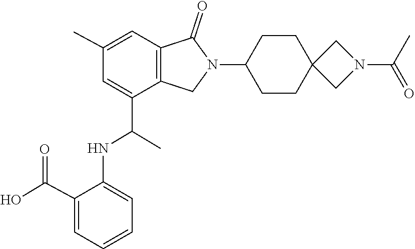 [SMILES: CC(=O)N1CC2(CCC(N3Cc4c(cc(C)cc4C(C)Nc4ccccc4C(=O)O)C3=O)CC2)C1]

### Step 1—Synthesis of tert-butyl 7-((2-bromo-6-chloro-4-methylbenzyl)amino)-2-azaspiro[3.5]nonane-2-carboxylate

A solution of (2-bromo-6-chloro-4-methyl-phenyl)methanamine (6.2 g, 26.44 mmol), tert-butyl 7-oxo-2-azaspiro[3.5]nonane-2-carboxylate (6.33 g, 26.44 mmol) and AcOH (1.51 mL, 26.44 mmol) in MeOH (60 mL) was stirred at room temperature for 30 min, and then NaBH₃CN (4.98 g, 79.31 mmol) was added. The mixture was stirred at room temperature for 16 h under N₂ atmosphere. The reaction was added water (100 mL) and extracted with EtOAc (200 mL×2). The combined organic layers were washed with brine (200 mL), dried over anhydrous Na₂SO₄, filtered and concentrated. The crude residue was purified by silica gel chromatography (solvent gradient: 0-100% EtOAc in petroleum ether) to give the title compound (10 g, 83%) as yellow oil. ¹H NMR (400 MHz, DMSO-d₆) δ 7.30 (s, 1H), 7.15 (s, 1H), 4.03 (s, 2H), 3.60 (s, 2H), 3.55 (s, 2H), 2.51-2.37 (m, 1H), 2.28 (s, 3H), 1.91-1.83 (m, 4H), 1.66-1.64 (m, 2H), 1.43 (s, 9H), 1.26-1.16 (m, 2H). MS: m/z 457.1 (M+H⁺).

### Step 2—Synthesis of tert-butyl 7-(4-chloro-6-methyl-1-oxoisoindolin-2-yl)-2-azaspiro[3.5]nonane-2-carboxylate

To a solution of tert-butyl 7-((2-bromo-6-chloro-4-methylbenzyl)amino)-2-azaspiro[3.5]nonane-2-carboxylate (2 g, 4.37 mmol) in DMF (10 mL) was added Pd(dppf)Cl₂ (959 mg, 1.31 mmol) and DIPEA (2.28 mL, 13.11 mmol). The mixture was stirred at 80° C. for 12 h under CO (50 Psi) atmosphere. After cooling to room temperature, the reaction was diluted with water (50 mL), extracted with EtOAc (100 mL×2). The combined organic layers were washed with brine (50 mL×3), dried over anhydrous Na₂SO₄, filtered and concentrated. The crude residue was purified by silica gel chromatography (solvent gradient: 0-20% EtOAc in petroleum ether) to give the title compound (1.59 g, 90%) as a yellow solid. MS: m/z 405.2 (M+H⁺).

### Step 3—Synthesis of tert-butyl 7-(4-acetyl-6-methyl-1-oxoisoindolin-2-yl)-2-azaspiro[3.5]nonane-2-carboxylate

A mixture of tert-butyl 7-(4-chloro-6-methyl-1-oxoisoindolin-2-yl)-2-azaspiro[3.5]nonane-2-carboxylate (1.59 g, 3.93 mmol), Sphos Pd G3 (306 mg, 393 μmol) and CsF (1.19 g, 7.85 mmol) in toluene (20 mL) was degassed and purged with N₂ for 3 times, and then tributyl(1-ethoxyvinyl)stannane (3.12 g, 8.64 mmol) was added, the reaction was stirred at 100° C. for 16 h under N₂ atmosphere. After cooling to room temperature, the reaction mixture was added 1 M HCl in water (6 mL) and stirred at room temperature for 0.5 h. The reaction mixture was added 30 mL 10% KF solution, stirred at room temperature over 2 h. The mixture was filtered and extracted with EtOAc (50 mL×3). The combined organic phase was washed with brine (30 mL), dried over anhydrous Na₂SO₄, filtered and concentrated in vacuo. The crude residue was purified by silica gel chromatography (solvent gradient: 0-50% EtOAc in petroleum ether) to give the title compound (1.5 g, 94%) as a white solid. MS: m/z 413.2 (M+H⁺).

### Step 4—Synthesis of tert-butyl 7-(4-(1-aminoethyl)-6-methyl-1-oxoisoindolin-2-yl)-2-azaspiro[3.5]nonane-2-carboxylate

To a solution of tert-butyl 7-(4-(1-aminoethyl)-6-methyl-1-oxoisoindolin-2-yl)-2-azaspiro[3.5]nonane-2-carboxylate (1.5 g, 3.64 mmol), NH₄OAc (5.61 g, 72.72 mmol) and AcOH (219 mg, 3.64 mmol) in MeOH (10 mL) was added NaBH₃CN (610 mg, 9.7 mmol). The mixture was stirred at 40° C. for 16 h under N₂ atmosphere. After cooling to room temperature, the reaction mixture was diluted with EtOAc (100 mL). The mixture was washed with saturated NaHCO₃ solution (50 mL×3), brine (50 mL), dried over anhydrous Na₂SO₄ and filtered. The filtrate was concentrated to give the title compound (1.5 g, crude) as a yellow solid that required no further purification. MS: m/z 414.2 (M+H⁺).

### Step 5—Synthesis of tert-butyl 7-(4-(1-((2-(methoxycarbonyl)phenyl)amino)ethyl)-6-methyl-1-oxoisoindolin-2-yl)-2-azaspiro[3.5]nonane-2-carboxylate

A mixture of tert-butyl 7-(4-(1-((2-(methoxycarbonyl)phenyl)amino)ethyl)-6-methyl-1-oxoisoindolin-2-yl)-2-azaspiro[3.5]nonane-2-carboxylate (1.48 g, 3.58 mmol), methyl 2-iodobenzoate (1.41 g, 5.37 mmol), Cs₂CO₃ (3.50 g, 10.74 mmol), Pd₂(dba)₃ (328 mg, 0.36 mmol) and Xantphos (414 mg, 0.72 mmol) in dioxane (15 mL) was stirred at 100° C. for 16 h under N₂ atmosphere. After cooling to room temperature, the reaction was filtered and the filtrate was concentrated. The crude residue was purified by silica gel chromatography (solvent gradient: 0-30% EtOAc in petroleum ether) to give the title compound (1.96 g, 53%) as yellow oil. MS: m/z 548.3 (M+H⁺).

### Step 6—Synthesis of methyl 2-((1-(6-methyl-1-oxo-2-(2-azaspiro[3.5]nonan-7-yl)isoindolin-4-yl)ethyl)amino)benzoate Trichloroacetate

A solution of tert-butyl 7-(4-(1-((2-(methoxycarbonyl)phenyl)amino)ethyl)-6-methyl-1-oxoisoindolin-2-yl)-2-azaspiro[3.5]nonane-2-carboxylate (206 mg, 376 mol) in DCM (3 mL) was added TFA (1 mL, 1.35 mmol). The mixture was stirred at room temperature for 1 h. The reaction was concentrated to afford the title compound (200 mg, crude) as yellow oil that required no further purification. MS: m/z 448.2 (M+H⁺).

### Step 7—Synthesis of methyl 2-((1-(2-(2-acetyl-2-azaspiro[3.5]nonan-7-yl)-6-methyl-1-oxoisoindolin-4-yl)ethyl)amino)benzoate

To a solution of methyl 2-((1-(6-methyl-1-oxo-2-(2-azaspiro[3.5]nonan-7-yl)isoindolin-4-yl)ethyl)amino)benzoate trichloroacetate (168 mg, 375 μmol) in DCM (2 mL) was added TEA (114 mg, 1.13 mmol). The mixture was stirred at 0° C. for 5 min, acetyl chloride (30 mg, 375 μmol) was added. The mixture was stirred at 0° C. for 1 h under N₂ atmosphere. The reaction was diluted with water (20 mL) and extracted with EtOAc (20 mL×3). The combined organic phase was washed with brine (20 mL×2), dried over anhydrous Na₂SO₄, filtered and concentrated to give the title compound (156 mg, crude) as a white solid that required no further purification. MS: m/z 490.3 (M+H⁺).

### Step 8—Synthesis of 2-((1-(2-(2-acetyl-2-azaspiro[3.5]nonan-7-yl)-6-methyl-1-oxoisoindolin-4-yl)ethyl)amino)benzoic Acid

To a solution of methyl 2-((1-(2-(2-acetyl-2-azaspiro[3.5]nonan-7-yl)-6-methyl-1-oxoisoindolin-4-yl)ethyl)amino)benzoate (40 mg, 82 mol) in H₂O (0.1 mL), MeOH (0.2 mL) and THF (4 mL) was added LiOH·H₂O (34 mg, 817 μmol). The mixture was stirred at 60° C. for 3 h.

After cooling to room temperature, the mixture was concentrated and the residue was purified by reverse phase chromatography (acetonitrile 39%-69%/0.225% formic acid in water) to give the title compound (2.5 mg, 6%) as a white solid. ¹H NMR (400 MHz, DMSO-d₆) δ 8.45 (s, 1H), 7.86-7.76 (m, 1H), 7.34 (s, 2H), 7.23-7.14 (m, 1H), 6.55-6.51 (m, 1H), 6.44-6.41 (m, 1H), 4.85-4.74 (m, 1H), 4.67-4.57 (m, 1H), 4.32-4.22 (m, 1H), 4.01-3.92 (m, 1H), 3.90-3.82 (m, 1H), 3.75 (s, 1H), 3.60 (s, 1H), 3.48 (s, 1H), 2.34 (s, 3H), 1.96-1.88 (m, 2H), 1.79-1.75 (m, 3H), 1.73-1.67 (m, 1H), 1.64-1.56 (m, 4H), 1.55-1.51 (m, 3H), 1.51-1.42 (m, 1H). MS: m/z 476.3 (M+H⁺).

### Compound 47: 6-chloro-3-((1-(2-(chroman-3-yl)-6-methyl-1-oxoisoindolin-4-yl)ethyl)amino)picolinic Acid

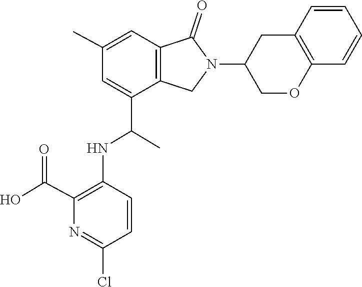 [SMILES: Cc1cc2c(c(C(C)Nc3ccc(Cl)nc3C(=O)O)c1)CN(C1COc3ccccc3C1)C2=O]

### Step 1—Synthesis of tert-butyl 6-chloro-3-((1-(2-(chroman-3-yl)-6-methyl-1-oxoisoindolin-4-yl)ethyl)amino)picolinate

A mixture of 4-(1-aminoethyl)-2-chroman-3-yl-6-methyl-isoindolin-1-one (50 mg, 155 mol), tert-butyl 3-bromo-6-chloropicolinate (68 mg, 233 μmol), Cs₂CO₃ (152 mg, 465 mol), Pd₂(dba)₃ (14 mg, 16 μmol) and Xantphos (18 mg, 31 μmol) in dioxane (2 mL) was stirred at 100° C. for 16 h under N₂ atmosphere. After cooling to room temperature, the reaction was filtered and the filtrate was concentrated. The crude residue was purified by silica gel chromatography (solvent gradient: 0-30% EtOAc in petroleum ether) to give the title compound (22 mg, 26%) as a yellow solid. MS: m/z 534.1 (M+H⁺).

### Step 2—Synthesis of 6-chloro-3-((1-(2-(chroman-3-yl)-6-methyl-1-oxoisoindolin-4-yl)ethyl)amino)picolinic Acid

To a solution of tert-butyl 6-chloro-3-((1-(2-(chroman-3-yl)-6-methyl-1-oxoisoindolin-4-yl)ethyl)amino)picolinate (62 mg, 116 μmol) in DCM (1 mL) was added TFA (0.5 mL, 6.7 mmol). The reaction mixture was stirred at room temperature for 16 h. The mixture was concentrated and the residue was purified by reverse phase chromatography (acetonitrile 47%-77%/0.225% formic acid in water) to give the title compound (28 mg, 35%) as a white solid. ¹H NMR (400 MHz, DMSO-d₆) δ 13.10 (s, 1H), 8.32-8.22 (m, 1H), 7.42 (s, 1H), 7.37-7.33 (m, 1H), 7.32-7.27 (m, 1H), 7.14-7.08 (m, 2H), 7.00-6.94 (m, 1H), 6.92-6.84 (m, 1H), 6.82-6.72 (m, 1H), 4.78-4.68 (m, 2H), 4.59-4.50 (m, 1H), 4.38-4.36 (m, 1H), 4.32-4.13 (m, 2H), 3.23-3.05 (m, 2H), 2.36 (s, 3H), 1.51 (d, J=5.2 Hz, 3H). MS: m/z 478.1 (M+H⁺).

### Compound 83: (2-((1-(6-methyl-2-(6-(oxetan-3-ylamino)chroman-3-yl)-1-oxoisoindolin-4-yl)ethyl)amino)benzoic Acid

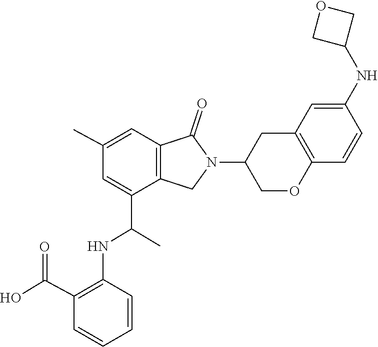 [SMILES: Cc1cc2c(c(C(C)Nc3ccccc3C(=O)O)c1)CN(C1COc3ccc(NC4COC4)cc3C1)C2=O]

### Step 1—Synthesis of 4-bromo-2-(6-chlorochroman-3-yl)-6-methylisoindolin-1-one

To a mixture of 6-chlorochroman-3-amine hydrochloride (376 mg, 1.71 mmol) and K₂CO₃ (858 mg, 6.21 mmol) in EtOH (9 mL) was added methyl 3-bromo-2-(bromomethyl)-5-methylbenzoate (500 mg, 1.55 mmol). After the addition, the reaction mixture was stirred at 60° C. for 16 h. After cooling to room temperature, the reaction was diluted with EtOAc (30 mL) and filtered, the filtrate was concentrated in vacuo. The crude residue was purified by silica gel chromatography (solvent gradient: 0-15% EtOAc in petroleum ether) to give the title compound (415 mg, 68%) as a white solid. ¹H NMR (400 MHz, DMSO-d₆) δ 7.66 (s, 1H), 7.56 (s, 1H), 7.21 (d, J=2.4 Hz, 1H), 7.19-7.16 (m, 1H), 6.88 (d, J=8.4 Hz, 1H), 4.63-4.57 (m, 1H), 4.34 (s, 2H), 4.31-4.26 (m, 2H), 3.27-3.05 (m, 2H), 2.41 (s, 3H). MS: m/z 392.0 (M+H⁺).

### Step 2—Synthesis of 2-(6-chlorochroman-3-yl)-4-(1-ethoxyvinyl)-6-methylisoindolin-1-one

A mixture of 4-bromo-2-(6-chlorochroman-3-yl)-6-methylisoindolin-1-one (415 mg, 1.06 mmol), tributyl(1-ethoxyvinyl)stannane (900 mg, 2.49 mmol) and Pd(dppf)Cl₂ (77 mg, 106 μmol) in dioxane (7 mL) was degassed with N₂ for 3 times and stirred at 90° C. for 16 h. After cooling to room temperature, the reaction was quenched with 10% KF solution (20 mL). The mixture was filtered and the filtrate was separated between EtOAc (30 mL) and H₂O (20 mL). The organic layer was washed with brine (20 mL), dried over anhydrous Na₂SO₄, filtered and concentrated in vacuo. The filtrate was concentrated to give the title compound (405 mg, crude) as a brown oil that required no further purification. MS: m/z 384.0 (M+H⁺).

### Step 3—Synthesis of 4-acetyl-2-(6-chlorochroman-3-yl)-6-methylisoindolin-1-one

To a solution of 2-(6-chlorochroman-3-yl)-4-(1-ethoxyvinyl)-6-methylisoindolin-1-one (405 mg, 1.06 mmol) in THF (6 mL) was added HCl (3 mL, 1 M in water) at room temperature. The reaction mixture was stirred at room temperature for 1 h. The mixture was quenched with saturated NaHCO₃ (20 mL), extracted with EtOAc (50 mL×2). The combined organic layers were washed with brine (20 mL), dried over anhydrous Na₂SO₄ and filtered. The crude residue was purified by silica gel chromatography (solvent gradient: 0-30% EtOAc in petroleum ether) to give the title compound (149 mg, 40%) as a yellow solid. MS: m/z 356.0 (M+H⁺).

### Step 4—Synthesis of 4-(1-aminoethyl)-2-(6-chlorochroman-3-yl)-6-methylisoindolin-1-one

To a solution of 4-(1-aminoethyl)-2-(6-chlorochroman-3-yl)-6-methylisoindolin-1-one (149 mg, 419 umol) and NH₄OAc (646 mg, 8.38 mmol) in MeOH (2 mL) was added NaBH₃CN (53 mg, 838 umol). The reaction was stirred at 60° C. for 16 h under N₂ atmosphere. After cooling to room temperature, the reaction mixture was diluted with EtOAc (50 mL). The mixture was washed with saturated NaHCO₃ solution (20 mL) and brine (20 mL), dried over anhydrous Na₂SO₄ and filtered. The filtrate was concentrated to give the title compound (148 mg, crude) as a yellow solid that required no further purification. MS: m/z 379.0 (M+Na⁺).

### Step 5—Synthesis of methyl 2-((1-(2-(6-chlorochroman-3-yl)-6-methyl-1-oxoisoindolin-4-yl)ethyl)amino)benzoate

A mixture of 4-(1-aminoethyl)-2-(6-chlorochroman-3-yl)-6-methylisoindolin-1-one (220 mg, 617 μmol), methyl 2-iodobenzoate (323 mg, 1.23 mmol), Cs₂CO₃ (603 mg, 1.85 mmol), Pd₂(dba)₃ (56 mg, 62 mol) and Xantphos (71 mg, 123 μmol) in dioxane (4 mL), the reaction mixture was stirred at 100° C. for 16 h under N₂ atmosphere. After cooling to room temperature, the reaction was filtered and the filtrate was concentrated. The crude residue was purified by silica gel chromatography (solvent gradient: 0-30% EtOAc in petroleum ether) to give the title compound (109 mg, 36%) as a yellow solid. MS: m/z 491.2 (M+H⁺).

### Step 6—Synthesis of methyl 2-((1-(6-methyl-2-(6-(oxetan-3-ylamino)chroman-3-yl)-1-oxoisoindolin-4-yl)ethyl)amino)benzoate

A mixture of methyl 2-((1-(2-(6-chlorochroman-3-yl)-6-methyl-1-oxoisoindolin-4-yl)ethyl)amino)benzoate (109 mg, 222 μmol), oxetan-3-amine (32 mg, 444 μmol), t-BuONa (26 mg, 266 mol), BrettPhos Pd G1 (18 mg, 22 mol) and RuPhos (21 mg, 44 mol) in dioxane (3 mL), the reaction mixture was stirred at 100° C. for 2 h under N₂ atmosphere. After cooling to room temperature, the reaction was filtered and the filtrate was concentrated. The crude residue was purified by silica gel chromatography (solvent gradient: 0-30% EtOAc in petroleum ether) to give the title compound (27 mg, 23%) as a white solid. ¹H NMR (400 MHz, DMSO-d₆) δ 8.11-8.03 (m, 1H), 7.81 (d, J=7.6 Hz, 1H), 7.40 (s, 1H), 7.36 (d, J=6.8 Hz, 1H), 7.26-7.20 (m, 1H), 6.67-6.54 (m, 2H), 6.43 (d, J=8.0 Hz, 1H), 6.36-6.27 (m, 1H), 6.23-6.15 (m, 1H), 5.95-5.87 (m, 1H), 4.83-4.77 (m, 2H), 4.75-4.69 (m, 2H), 4.55-4.32 (m, 5H), 4.23-4.08 (m, 2H), 3.86-3.84 (m, 3H), 3.18-3.09 (m, 1H), 3.02-2.89 (m, 1H), 2.35 (s, 3H), 1.54-1.49 (m, 3H). MS: m/z 550.1 (M+Na⁺).

### Step 7—Synthesis of 2-((1-(6-methyl-2-(6-(oxetan-3-ylamino)chroman-3-yl)-1-oxoisoindolin-4-yl)ethyl)amino)benzoic Acid

A solution of methyl 2-((1-(6-methyl-2-(6-(oxetan-3-ylamino)chroman-3-yl)-1-oxoisoindolin-4-yl)ethyl)amino)benzoate (27 mg, 51 mol) in EtOH (0.5 mL) and H₂O (0.5 mL) was added KOH (29 mg, 512 mol). The mixture was stirred at 100° C. for 0.5 h. After cooling to room temperature, the reaction mixture was adjusted to pH 6 with formic acid. The reaction mixture was concentrated and the residue was purified by reverse phase chromatography (acetonitrile 30%-60%/0.225% formic acid in water) to give the title compound (3.78 mg, 14%) as a white solid. ¹H NMR (400 MHz, DMSO-d₆) δ 8.48 (s, 1H), 7.79 (d, J=8.0 Hz, 1H), 7.38 (s, 1H), 7.36-7.34 (m, 1H), 7.19-7.10 (m, 1H), 6.65-6.60 (m, 1H), 6.54-6.47 (m, 1H), 6.42-6.27 (m, 2H), 6.23-6.16 (m, 1H), 5.94-5.86 (m, 1H), 4.85-4.75 (m, 2H), 4.75-4.62 (m, 2H), 4.54-4.31 (m, 5H), 4.23-3.93 (m, 2H), 3.14-3.01 (m, 1H), 3.00-2.86 (m, 1H), 2.35 (s, 3H), 1.49 (d, J=5.2 Hz, 3H). MS: m/z 536.1 (M+Na⁺).

### Compound 53: 2-((1-(6-chloro-2-(2,3-dihydro-1H-inden-2-yl)-1-oxoisoindolin-4-yl)ethyl)amino)benzoic Acid

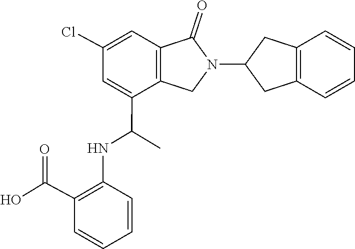 [SMILES: CC(Nc1ccccc1C(=O)O)c1cc(Cl)cc2c1CN(C1Cc3ccccc3C1)C2=O]

### Step 1—Synthesis of (2-bromo-4-chlorophenyl)methanamine

A mixture of 2-bromo-4-chloro-benzonitrile (10 g, 46.20 mmol) in THF (150 mL) was added BH₃·THF (46.20 mL, 46.20 mmol 1 M in THF) at room temperature under N₂ atmosphere. The resulting mixture was stirred at 80° C. for 1 h. After cooling to 0° C., 1 M HCl (46 mL) was added dropwise and then added MeOH (30 mL). The mixture was adjusted to pH 8 with 1 M NaOH aqueous solution, extracted with DCM (200 mL×3). The combined organic layers were washed with brine (200 mL), dried over anhydrous Na₂SO₄, filtered and concentrated in vacuo. The crude residue was purified by silica gel chromatography (solvent gradient: 0-30% EtOAc in petroleum ether) to give the title compound (8 g, 53%) as yellow oil. ¹H NMR (400 MHz, CDCl₃) δ 7.57 (d, J=2.0 Hz, 1H), 7.35 (d, J=8.0 Hz, 1H), 7.31-7.27 (m, 1H), 3.89 (s, 2H) MS: m/z 219.7 (M+H⁺).

### Step 2—Synthesis of N-(2-bromo-4-chlorobenzyl)-2,3-dihydro-1H-inden-2-amine

To a solution of indan-2-one (3.24 g, 24.49 mmol) and (2-bromo-4-chloro-phenyl)methanamine (6 g, 27.21 mmol) in DCM (30 mL) was added NaBH₃CN (5.13 g, 81.63 mmol). The reaction mixture was stirred at room temperature for 16 h under N₂ atmosphere. The reaction mixture was diluted with H₂O (200 mL) and extracted with EtOAc (100 mL×2). The combined organic layers were washed with brine (100 mL×3), dried over anhydrous Na₂SO₄, filtered and concentrated in vacuo. The crude residue was purified by silica gel chromatography (solvent gradient: 0-3% EtOAc in petroleum ether) to give the title compound (5.97 g, 65%) as yellow oil. ¹H NMR (400 MHz, CDCl₃) δ 7.57 (d, J=2.0 Hz, 1H), 7.39 (d, J=8.4 Hz, 1H), 7.28-7.26 (m, 2H), 7.24-7.18 (m, 2H), 7.18-7.13 (m, 2H), 3.91 (s, 2H), 3.68-3.64 (m, 1H), 3.22-3.16 (m, 2H), 2.87-2.81 (m, 2H) MS: m/z 335.9 (M+H⁺).

### Step 3—Synthesis of ethyl (2-bromo-4-chlorobenzyl)(2,3-dihydro-1H-inden-2-yl)carbamate

To a solution of N-(2-bromo-4-chlorobenzyl)-2,3-dihydro-1H-inden-2-amine (5.97 g, 17.73 mmol) in DCM (80 mL) was added TEA (7.40 mL, 53.20 mmol), and then ethyl carbonochloridate (4.15 g, 38.24 mmol, 3.7 mL) was added dropwise at 0° C. The mixture was stirred at room temperature for 16 h. The resulting mixture was quenched with water (50 mL) and sat. aq. NaHCO₃ solution (50 mL), extracted with DCM (100 mL×2). The combined organic layer was washed with brine (100 mL), dried over anhydrous Na₂SO₄, filtered and concentrated in vacuo. The crude residue was purified by silica gel chromatography (solvent gradient: 0-3% EtOAc in petroleum ether) to give the title compound (6.33 g, 87%) as yellow oil. ¹H NMR (400 MHz, CDCl₃) δ 7.55 (d, J=2.0 Hz, 1H), 7.29-7.34 (m, 1H), 7.17-7.13 (m, 5H), 5.22-4.85 (m, 1H), 4.48 (s, 2H), 4.28-4.09 (m, 2H), 3.17-3.04 (m, 2H), 2.96-2.89 (m, 2H), 1.27-1.14 (m, 3H). MS: m/z 407.9 (M+H⁺).

### Step 4—Synthesis of 4-bromo-6-chloro-2-(2,3-dihydro-1H-inden-2-yl)isoindolin-1-one

To a solution of ethyl (2-bromo-4-chlorobenzyl)(2,3-dihydro-1H-inden-2-yl)carbamate (3.33 g, 8.15 mmol) in DCM (100 mL) was added P₂O₅ (11.56 g, 81.47 mmol) at 0° C. under N₂ atmosphere. The mixture was stirred at room temperature for 16 h. The reaction was quenched with 1 M NaOH solution carefully, and the mixture was extracted with DCM (100 mL×2). The combined organics were dried over Na₂SO₄, filtered and concentrated in vacuo. The crude residue was purified by reverse phase chromatography (acetonitrile 61%-91%/0.225% formic acid in water) to give the title compound (500 mg, 11%) as a white solid. ¹H NMR (400 MHz, DMSO-d₆) δ 7.97 (d, J=1.6 Hz, 1H), 7.74 (d, J=1.2 Hz, 1H), 7.31-7.24 (m, 2H), 7.23-7.17 (m, 2H), 5.13-5.04 (m, 1H), 4.24 (s, 2H), 3.28-3.20 (m, 2H), 3.18-3.11 (m, 2H). MS: m/z 361.8 (M+H⁺).

### Step 5—Synthesis of 6-chloro-2-(2,3-dihydro-1H-inden-2-yl)-4-(1-ethoxyvinyl)isoindolin-1-one

A mixture of 4-bromo-6-chloro-2-(2,3-dihydro-1H-inden-2-yl)isoindolin-1-one (565 mg, 1.56 mmol), Pd(dppf)Cl₂ (114 mg, 156 μmol) in dioxane (6 mL) was degassed and purged with N₂ for 3 times, and then tributyl(1-ethoxyvinyl)stannane (730 mg, 2.02 mmol) was added, the final mixture was stirred at 90° C. for 16 h under N₂ atmosphere. After cooling to room temperature, the reaction was quenched with 10% KF solution (20 mL). The mixture was filtered and the filtrate was separated between EtOAc (50 mL) and H₂O (30 mL). The organic layer was washed with brine (30 mL), dried over anhydrous Na₂SO₄, filtered and concentrated in vacuo to give the title compound (551 mg, crude) as yellow oil that required no further purification. MS: m/z 353.9 (M+H⁺).

### Step 6—Synthesis of 4-acetyl-6-chloro-2-(2,3-dihydro-1H-inden-2-yl)isoindolin-1-one

To a solution of 6-chloro-2-(2,3-dihydro-1H-inden-2-yl)-4-(1-ethoxyvinyl)isoindolin-1-one (551 mg, 1.56 mmol) in THF (5 mL) was added HCl (1 mL, 1 M in water) at room temperature. The reaction mixture was stirred at room temperature for 1 h. The mixture was quenched with saturated NaHCO₃ (10 mL), extracted with EtOAc (30 mL×2). The combined organic layers were washed with brine (20 mL), dried over anhydrous Na₂SO₄ and filtered. The crude residue was purified by silica gel chromatography (solvent gradient: 0-20% EtOAc in petroleum ether) to give the title compound (390 mg, 77%) as a yellow solid. ¹H NMR (400 MHz, DMSO-d₆) δ 8.26 (d, J=1.2 Hz, 1H), 7.95 (d, J=1.2 Hz, 1H), 7.30-7.26 (m, 2H), 7.23-7.18 (m, 2H), 5.13-5.05 (m, 1H), 4.52 (s, 2H), 3.29-3.23 (m, 2H), 3.15-3.08 (m, 2H), 2.61 (s, 3H). MS: m/z 325.9 (M+H⁺).

### Step 7—Synthesis of 4-(1-aminoethyl)-6-chloro-2-(2,3-dihydro-1H-inden-2-yl)isoindolin-1-one

A mixture of 4-acetyl-6-chloro-2-(2,3-dihydro-1H-inden-2-yl)isoindolin-1-one (380 mg, 1.17 mmol), NH₄OAc (1.35 g, 17.50 mmol) and HOAc (70 mg, 1.17 mmol) in MeOH (15 mL) was stirred at room temperature for 1 h. Then NaBH₃CN (220 mg, 3.50 mmol) was added to the reaction mixture. After the addition, the reaction mixture was stirred at 60° C. for 16 h. After cooling to room temperature, the reaction mixture was diluted with DCM (50 mL), washed with saturated NaHCO₃ solution (20 mL×3), brine (20 mL), dried over anhydrous Na₂SO₄ and filtered. The filtrate was concentrated to give the title compound (490 mg, crude) as a yellow solid that required no further purification. MS: m/z 348.9 (M+Na⁺).

### Step 8—Synthesis of tert-butyl 2-((1-(6-chloro-2-(2,3-dihydro-1H-inden-2-yl)-1-oxoisoindolin-4-yl)ethyl)amino)benzoate

A mixture of 4-(1-aminoethyl)-6-chloro-2-(2,3-dihydro-1H-inden-2-yl)isoindolin-1-one (450 mg, 1.38 mmol), tert-butyl 2-iodobenzoate (628 mg, 2.07 mmol), Cs₂CO₃ (897 mg, 2.75 mmol), Pd₂(dba)₃ (126 mg, 138 μmol) and Xantphos (159 mg, 275 mol) in dioxane (8 mL) was stirred at 100° C. for 16 h under N₂ atmosphere. After cooling to room temperature, the reaction was filtered and the filtrate was concentrated. The crude residue was purified by silica gel chromatography (solvent gradient: 0-15% EtOAc in petroleum ether) to give the title compound (350 mg, 50%) as a yellow solid. MS: m/z 525.1 (M+Na⁺).

### Step 9—Synthesis of 2-((1-(6-chloro-2-(2,3-dihydro-1H-inden-2-yl)-1-oxoisoindolin-4-yl)ethyl)amino)benzoic Acid

To a solution of tert-butyl 2-((1-(6-chloro-2-(2,3-dihydro-1H-inden-2-yl)-1-oxoisoindolin-4-yl)ethyl)amino)benzoate (100 mg, 199 μmol) in DCM (2 mL) was added TFA (0.8 mL, 10.77 mmol). The reaction mixture was stirred at 40° C. for 2 h. After cooling to room temperature, the reaction mixture was concentrated and the residue was purified by reverse phase chromatography (acetonitrile 58%-88%/0.225% formic acid in water) to give the title compound (73 mg, 82%) as a white solid. ¹H NMR (400 MHz, DMSO-d₆) δ 12.79 (s, 1H), 8.25 (s, 1H), 7.82-7.78 (m, 1H), 7.57-7.53 (m, 2H), 7.28-7.22 (m, 2H), 7.22-7.16 (m, 3H), 6.58-6.54 (m, 1H), 6.41 (d, J=8.4 Hz, 1H), 5.08-5.02 (m, 1H), 4.83-4.72 (m, 2H), 4.41-4.36 (m, 1H), 3.25-3.20 (m, 2H), 3.19-3.09 (m, 2H), 1.53 (d, J=6.8 Hz, 3H). MS: m/z 447.1 (M+H⁺).

### Compound 102: 2-((1-(2-(1-(2-oxabicyclo[3.1.1]heptane-1-carbonyl)azepan-4-yl)-6-methyl-1-oxoisoindolin-4-yl)ethyl)amino)benzoic Acid

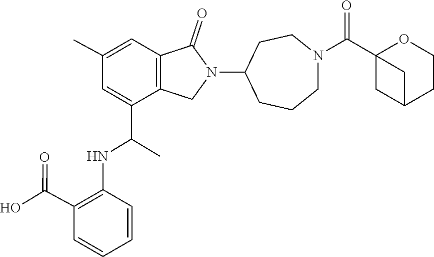 [SMILES: Cc1cc2c(c(C(C)Nc3ccccc3C(=O)O)c1)CN(C1CCCN(C(=O)C34CC(CCO3)C4)CC1)C2=O]

### Step 1—Synthesis of methyl 2-((1-(2-(1-(2-oxabicyclo[3.1.1]heptane-1-carbonyl)azepan-4-yl)-6-methyl-1-oxoisoindolin-4-yl)ethyl)amino)benzoate

To a solution of methyl 2-((1-(2-(azepan-4-yl)-6-methyl-1-oxoisoindolin-4-yl)ethyl)amino)benzoate hydrochloride (150 mg, 356 mol), 2-oxabicyclo[3.1.1]heptane-1-carboxylic acid (61 mg, 427 μmol) and DIEA (138 mg, 1.07 mmol) in DMF (3 mL) was added HATU (162 mg, 427 mol). The mixture was stirred at room temperature for 1 h. The reaction was quenched with H₂O (10 mL), extracted with EtOAc (10 mL×2). The combined organic layers were washed with brine (10 mL×3), dried over anhydrous Na₂SO₄, filtered and concentrated in vacuo. The residue was purified by silica gel chromatography (solvent gradient: 0-30% EtOAc in petroleum ether) to give the title compound (145 mg, 75%) as colorless oil. ¹H NMR (400 MHz, DMSO-d₆) δ 8.13-8.06 (m, 1H), 7.81 (d, J=8.0 Hz, 1H), 7.39-7.29 (m, 2H), 7.28-7.19 (m, 1H), 6.61-6.53 (m, 1H), 6.49 (d, J=7.6 Hz, 1H), 4.86-4.76 (m, 1H), 4.69-4.57 (m, 1H), 4.34-4.20 (m, 1H), 4.19-4.11 (m, 1H), 4.10-3.98 (m, 4H), 3.90-3.82 (m, 3H), 3.81-3.45 (m, 4H), 3.24-3.07 (m, 1H), 2.43-2.40 (m, 1H), 2.35-2.32 (m, 3H), 2.31-2.18 (m, 2H), 2.07-2.01 (m, 2H), 1.98-1.62 (m, 8H), 1.58-1.50 (m, 3H). MS: m/z 568.2 (M+Na⁺).

### Step 2—Synthesis of 2-((1-(2-(1-(2-oxabicyclo[3.1.1]heptane-1-carbonyl)azepan-4-yl)-6-methyl-1-oxoisoindolin-4-yl)ethyl)amino)benzoic Acid

To a solution of methyl 2-((1-(2-(1-(2-oxabicyclo[3.1.1]heptane-1-carbonyl)azepan-4-yl)-6-methyl-1-oxoisoindolin-4-yl)ethyl)amino)benzoate (20 mg, 37 μmol) in EtOH (0.5 mL) and H₂O (0.5 mL) was added KOH (21 mg, 366 mol). The reaction mixture was stirred at 100° C. for 1 h. After cooling to room temperature, the mixture was acidified to pH 3 with 1 M HCl, diluted with H₂O (10 mL), extracted with EtOAc (10 mL×2). The combined organic layers were washed with brine (10 mL), dried over Na₂SO₄ and filtered and concentrated in vacuo. The residue was purified by prep_TLC (DCM/MeOH=9/1) to give the title compound (4.38 mg, 22%) as a white solid. ¹H NMR (400 MHz, DMSO-d₆) δ 12.76 (s, 1H), 8.32 (s, 1H), 7.79 (d, J=7.6 Hz, 1H), 7.38-7.27 (m, 2H), 7.23-7.15 (m, 1H), 6.57-6.49 (m, 1H), 6.48-6.39 (m, 1H), 4.84-4.72 (m, 1H), 4.70-4.56 (m, 1H), 4.42-4.23 (m, 1H), 4.20-4.11 (m, 1H), 4.10-4.01 (m, 2H), 3.84-3.61 (m, 1H), 3.60-3.40 (m, 2H), 3.25-3.14 (m, 1H), 2.45-2.38 (m, 1H), 2.37-2.31 (m, 3H), 2.30-2.14 (m, 2H), 2.05-1.64 (m, 10H), 1.57-1.50 (m, 3H). MS: m/z 532.3 (M+H⁺).

### Compound 141: 2-((1-(2-(chroman-4-yl)-6-methyl-1-oxoisoindolin-4-yl)ethyl)amino)benzoic Acid

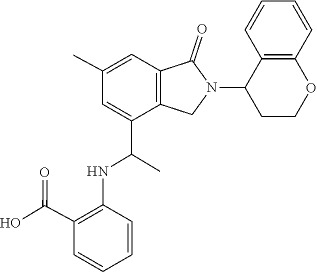 [SMILES: Cc1cc2c(c(C(C)Nc3ccccc3C(=O)O)c1)CN(C1CCOc3ccccc31)C2=O]

### Step 1—Synthesis of 4-bromo-2-(chroman-4-yl)-6-methylisoindolin-1-one

To a solution of methyl 3-bromo-2-(bromomethyl)-5-methylbenzoate (1.30 g, 4.02 mmol) and chroman-4-amine (723 mg, 4.84 mmol) in EtOH (15 mL) was added K₂CO₃ (1.67 g, 12.09 mmol) at room temperature. The reaction was stirred at 60° C. for 16 h under N₂ atmosphere. After cooling to room temperature, the reaction was quenched with water (30 mL), extracted with EtOAc (50 mL×2). The combined organic layers were washed with brine (30 mL), dried over anhydrous Na₂SO₄, filtered and concentrated in vacuo. The crude residue was purified by silica gel chromatography (solvent gradient: 0-2% EtOAc in petroleum ether) to give the title compound (730 mg, 50%) as yellow oil. ¹H NMR (400 MHz, DMSO-d₆) δ 7.69 (s, 1H), 7.62 (s, 1H), 7.20-7.14 (m, 1H), 6.90-6.81 (m, 3H), 5.63-5.55 (m, 1H), 4.46-4.24 (m, 3H), 3.88-3.83 (m, 1H), 2.43 (s, 3H), 2.39-2.29 (m, 1H), 2.12-2.09 (m, 1H). MS: m/z 357.9 (M+H⁺).

### Step 2—Synthesis of 4-acetyl-2-(chroman-4-yl)-6-methylisoindolin-1-one

To a solution of 4-bromo-2-(chroman-4-yl)-6-methylisoindolin-1-one (730 mg, 2.04 mmol) and tributyl(1-ethoxyvinyl)stannane (1.77 g, 4.90 mmol) in dioxane (15 mL) was added Pd(dppf)Cl₂ (149 mg, 204 umol). The reaction was stirred at 90° C. for 16 h under N₂ atmosphere. After cooling to room temperature, the reaction mixture was added 1 M HCl in water (4 mL) and stirred at room temperature for 0.5 h. The reaction mixture was added 30 mL 10% KF solution, stirred at room temperature over 2 h. The mixture was filtered and extracted with EtOAc (30 mL×3). The combined organic phase was washed with brine (30 mL), dried over anhydrous Na₂SO₄, filtered and concentrated in vacuo. The crude residue was purified by silica gel chromatography (solvent gradient: 0-10% EtOAc in petroleum ether) to give the title compound (488 mg, 68%) as yellow oil. ¹H NMR (400 MHz, DMSO-d₆) δ 7.94 (s, 1H), 7.86 (s, 1H), 7.18-7.12 (m, 1H), 6.91-6.85 (m, 2H), 6.82-6.77 (m, 1H), 5.82-5.81 (m, 1H), 4.64-4.62 (m, 1H), 4.43-4.38 (m, 2H), 4.34-4.25 (m, 1H), 2.61 (s, 3H), 2.55 (s, 3H), 2.43-2.31 (m, 1H), 2.28-2.18 (m, 1H). MS: m/z 322.0 (M+H⁺).

### Step 3—Synthesis of 4-(1-aminoethyl)-2-(chroman-4-yl)-6-methylisoindolin-1-one

A mixture of 4-acetyl-2-(chroman-4-yl)-6-methylisoindolin-1-one (200 mg, 622 mol), NH₄OAc (720 mg, 9.34 mmol) and AcOH (4 mg, 6.5 μmol) in MeOH (10 mL) was stirred at room temperature for 0.5 h. Then NaBH₃CN (117 mg, 1.87 mol) was added to the reaction mixture. After the addition, the reaction mixture was stirred at 60° C. for 16 h. After cooling to room temperature, the reaction mixture was diluted with DCM (50 mL). The mixture was washed with saturated NaHCO₃ solution (20 mL×3), brine (20 mL), dried over anhydrous Na₂SO₄ and filtered. The filtrate was concentrated to give the title compound (250 mg, crude) as a brown solid that required no further purification. MS: m/z 345 (M+Na⁺).

### Step 5—Synthesis of tert-butyl 2-((1-(2-(chroman-4-yl)-6-methyl-1-oxoisoindolin-4-yl)ethyl)amino)benzoate

A mixture of 4-(1-aminoethyl)-2-(chroman-4-yl)-6-methylisoindolin-1-one (250 mg, 775 mol), tert-butyl 2-iodobenzoate (236 mg, 775 mol), Pd₂(dba)₃ (71 mg, 78 mol), Xantphos (90 mg, 155 μmol) and Cs₂CO₃ (758 mg, 2.33 mmol) in dioxane (10 mL). The reaction mixture was stirred at 100° C. for 16 h under N₂ atmosphere. After cooling to room temperature, the reaction was filtered and the filtrate was concentrated. The crude residue was purified by silica gel chromatography (solvent gradient: 0-15% EtOAc in petroleum ether) to give the title compound (165 mg, 43%) as colorless oil. ¹H NMR (400 MHz, DMSO-d₆) δ 8.07 (d, J=4.4 Hz, 1H), 7.86-7.76 (m, 1H), 7.61 (s, 1H), 7.35 (s, 1H), 7.20-7.06 (m, 2H), 6.87-6.81 (m, 1H), 6.68-6.65 (m, 1H), 6.52 (d, J=7.6 Hz, 1H), 6.26-6.20 (m, 1H), 5.79-5.75 (m, 1H), 4.77-4.73 (m, 1H), 4.56-4.51 (m, 1H), 4.38-4.26 (m, 3H), 4.22-4.18 (m, 1H), 2.44 (s, 3H), 2.35-2.13 (m, 2H), 1.59-1.53 (m, 12H). MS: m/z 499.1 (M+H⁺).

### Step 6—Synthesis of 2-((1-(2-(chroman-4-yl)-6-methyl-1-oxoisoindolin-4-yl)ethyl)amino)benzoic Acid

To a mixture of tert-butyl 2-((1-(2-(chroman-4-yl)-6-methyl-1-oxoisoindolin-4-yl)ethyl)amino)benzoate (30 mg, 60 mol) in DCM (2 mL) was added TFA (1 mL, 13.5 mmol). The reaction mixture was stirred at room temperature for 16 h. The reaction mixture was concentrated and the residue was purified by reverse phase chromatography (acetonitrile 50%-80%/0.225% formic acid in water) to give the title compound (2.6 mg, 10%) as a white solid. ¹H NMR (400 MHz, DMSO-d₆) δ 8.34 (s, 1H), 7.79-7.78 (m, 1H), 7.47-7.43 (m, 1H), 7.41-7.35 (m, 1H), 7.20-7.09 (m, 2H), 6.87-6.81 (m, 2H), 6.73-6.64 (m, 1H), 6.53-6.46 (m, 1H), 6.40-6.31 (m, 1H), 5.66-5.50 (m, 1H), 4.70-4.66 (m, 1H), 4.44-4.25 (m, 3H), 4.23-3.87 (m, 1H), 2.39-2.35 (m, 3H), 2.26-2.07 (m, 2H), 1.46-1.42 (m, 3H). MS: m/z 443.1 (M+H⁺).

The rest of the compounds in Table 1 were prepared using procedures analogous to those disclosed above with appropriate modifications within the purview of one skilled in the art.

### Example 2: Biological Assays

### PI3Kα Cellular Assay Experimental Procedure:

SKBR3 or T47D cells are seeded in DMEM containing 10% FBS at 25k cells/well into 96-well cell culture format. Cells are incubated overnight at 37° C. in a 5% CO₂ incubator and the following day cell media is aspirated, adherent cells are washed 1× with room temperature PBS prior to serum-free media application. Cells are returned to 37° C. 5% CO₂ incubator and incubated a further 16 hrs. Compounds are added to serum starved adherent cells with a top dose of 10,000 nM and 3× multiple dose reductions for a minimum dose of 0.5 nM diluted in DMSO. Cell/compound incubation continues for 1 hr in a 37° C., 5% CO₂ incubator prior to 10 minute PIK3CA stimulation with 20 ng/ml EGF. Cells treated with 0.1% DMSO and 20 ng/mL EGF are employed as negative control, cells treated with 10 uM Alpelisib and 20 ng/mL EGF are employed as a positive control. After 10 mins, plates are removed from incubator and cells are lysed with buffer and shaking 45 minutes. 20 μL of lysate is transferred to an opti-384 plate and 2.5 pd of Phospho-AKT d2 antibody with 2.5 μl of Phospho-AKT Eu Cryptate solution in the detection buffer are added to each well. 384 well plate is left overnight at room temperature before reading HTRF on an Envision plate reader.

The biological activity of certain compounds using the assays described above is shown in Table 2. The ranges are as follows:
for T47D pAKT IC₅₀ (nM): A denotes <750 nM; B denotes 750 nM≤IC₅₀<2,000 nM; C denotes ≥2,000 nM. ND denotes value not determined with that assay for the specified compound; and
for T47D (H1047R) selectivity over SKBR3 (WT): A denotes >20-fold; B denotes 20-fold ≥value >5-fold; C denotes ≤5-fold. ND denotes value not determined with that assay for the specified compound; ND denotes value not determined with that assay for the specified compound.

TABLE 2

| | T47D pAKT S473 | T47D pAKT S473 HTRF IC50 |
|---|---|---|
| Compound | HTRF IC50 (H1047R): | (H1047R): T47D (H1047R) |
| Number | Average IC50 (nM) | selectivity over SKBR3 (WT) |
| 1 | A | A |
| 2 | A | C |
| 3 | A | C |
| 4 | A | C |
| 5 | B | C |
| 6 | B | C |
| 7 | B | C |
| 8 | B | C |
| 9 | A | B |
| 10 | A | B |
| 11 | A | B |
| 12 | B | B |
| 13 | B | B |
| 14 | A | B |
| 15 | A | A |
| 16 | A | A |
| 17 | B | B |
| 18 | B | A |
| 19 | B | A |
| 20 | B | A |
| 21 | A | B |
| 22 | A | B |
| 23 | A | B |
| 24 | A | B |
| 25 | A | A |
| 26 | B | A |
| 27 | B | A |
| 28 | A | B |
| 29 | A | A |
| 30 | A | A |
| 31 | B | B |
| 32 | B | B |
| 33 | B | A |
| 34 | B | B |
| 35 | A | B |
| 36 | A | B |
| 37 | A | B |
| 38 | A | A |
| 39 | A | A |
| 40 | A | A |
| 41 | A | B |
| 42 | A | A |
| 43 | A | A |
| 44 | A | C |
| 45 | A | C |
| 46 | A | C |
| 47 | A | C |
| 48 | A | C |
| 49 | A | C |
| 50 | C | C |
| 51 | C | C |
| 52 | C | C |
| 53 | C | C |
| 54 | C | C |
| 55 | C | C |
| 56 | C | A |
| 57 | C | A |
| 58 | C | A |
| 59 | C | A |
| 60 | C | A |
| 61 | C | A |
| 62 | A | A |
| 63 | A | A |
| 64 | A | A |
| 65 | A | A |
| 66 | A | A |
| 67 | A | A |
| 68 | A | C |
| 69 | A | C |
| 70 | A | C |
| 71 | A | C |
| 72 | A | C |
| 73 | A | C |
| 74 | A | C |
| 75 | A | C |
| 76 | A | C |
| 77 | C | C |
| 78 | C | C |
| 79 | C | C |
| 80 | C | A |
| 81 | C | A |
| 82 | C | A |
| 83 | C | A |
| 84 | C | A |
| 85 | C | A |
| 86 | C | A |
| 87 | C | A |
| 88 | C | A |
| 89 | A | A |
| 90 | A | A |
| 91 | A | A |
| 92 | A | A |
| 93 | A | A |
| 94 | A | A |
| 95 | A | C |
| 96 | A | C |
| 97 | A | C |
| 98 | A | C |
| 99 | A | C |
| 100 | A | C |
| 101 | C | C |
| 102 | C | C |
| 103 | C | C |
| 104 | C | C |
| 105 | C | C |
| 106 | C | C |
| 107 | C | A |
| 108 | C | A |
| 109 | C | A |
| 110 | C | A |
| 111 | C | A |
| 112 | C | A |
| 113 | A | A |
| 114 | A | A |
| 115 | A | A |
| 116 | A | A |
| 117 | A | A |
| 118 | A | A |
| 119 | A | A |
| 120 | A | A |
| 121 | A | A |
| 122 | A | B |
| 123 | A | B |
| 124 | A | B |
| 125 | A | B |
| 126 | A | A |
| 127 | A | A |
| 128 | A | A |
| 129 | A | B |
| 130 | A | B |
| 131 | A | A |
| 132 | A | A |
| 133 | A | A |
| 134 | B | A |
| 135 | B | A |
| 136 | A | B |
| 137 | A | B |
| 138 | A | B |
| 139 | B | B |
| 140 | A | A |
| 141 | A | A |
| 142 | B | A |
| 143 | B | B |
| 144 | B | B |
| 145 | B | A |
| 146 | B | A |
| 147 | B | A |
| 148 | C | B |
| 149 | C | B |
| 150 | C | B |
| 151 | C | B |
| 152 | C | A |
| 153 | C | A |
| 154 | C | A |
| 155 | A | A |

### Embodiments

Embodiment 1. A compound of Formula (I):

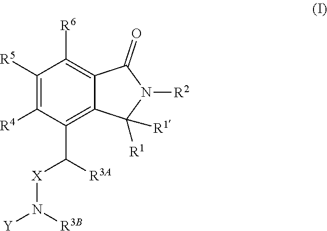

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1′} is hydrogen or C1-C6 alkyl; or R¹ and R^{1′}, together with the carbon atom to which they are attached form a C3-C10 cycloalkyl;
R² is C6-C12 aryl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, C4-C10 cycloalkyl optionally substituted with 1-4 independently selected R^{2A}, C1-C6 alkoxyalkyl optionally substituted with —C(═O)NR^{A}R^{C}, C1-C6 aralkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

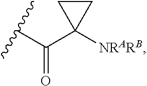

(vii) —NHC(═O)R^{C},
(viii) —C(═O)R^{D},
(ix) —C(═O)OR^{E},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl, —C(═O)NR^{A}R^{B}, C1-C6 haloalkyl, and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl,
(xx) C3-C6 cycloalkyl optionally substituted with 4-10 membered heterocyclyl optionally substituted with C1-C6 alkyl;
(xxi) C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}; and
(xxii) 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B};
each R^{A} and R^{B} is independently selected from hydrogen, hydroxyl, C1-C6 alkoxy, C3-C6 cycloalkyl, C2-C6 alkenyl, cyano, 4-10 membered heterocyclyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, hydroxyl, C1-C6 alkyl, and —C(═O)C1-C6 alkyl;
each R^{C} is independently selected from C3-C6 cycloalkyl, —C(═O)NHR^{Y1}, C2-C6 alkenyl, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, phenyl, 4-10 membered heterocyclyl, —NR^{A}R^{B}, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, halogen, C1-C6 haloalkyl, C3-C6 cycloalkyl, cyano, —NR^{5A}R^{5B}, —NR⁵AC(═O)R^{5B}, or —C(═O)NR^{5A}R^{5B},
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

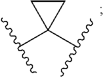 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Embodiment 2. The compound of Embodiment 1, wherein R¹ is hydrogen.

Embodiment 3. The compound of Embodiment 1, wherein R¹ cyano.

Embodiment 4. The compound of Embodiment 1, wherein R¹ is C3-C6 cycloalkyl.

Embodiment 5. The compound of Embodiment 1, wherein R¹ is C1-C6 alkyl optionally substituted with phenyl optionally substituted with halogen.

Embodiment 6. The compound of Embodiment 1, wherein R¹ is C1-C6 alkyl substituted with phenyl optionally substituted with halogen.

Embodiment 7. The compound of Embodiment 1 or 6, wherein R¹ is C1-C6 alkyl substituted with phenyl substituted with halogen.

Embodiment 8. The compound of Embodiment 1 or 6, wherein R¹ is C1-C6 alkyl substituted with phenyl.

Embodiment 9. The compound of Embodiment 1 or 6, wherein R¹ is C1-C6 alkyl.

Embodiment 10. The compound of any one of Embodiments 1, 6, or 9, wherein R¹ is C1-C3 alkyl.

Embodiment 11. The compound of Embodiment 1, wherein R¹ is C1-C6 thioalkyl.

Embodiment 12. The compound of Embodiment 1, wherein R¹ is C1-C6 haloalkyl.

Embodiment 13. The compound of Embodiment 1, wherein R¹ is C1-C6 alkoxy.

Embodiment 14. The compound of Embodiment 1, wherein R¹ is C1-C6 alkoxyalkyl.

Embodiment 15. The compound of any one of Embodiments 1-14, wherein R^{1′} is hydrogen.

Embodiment 16. The compound of any one of Embodiments 1-14, wherein R^{1′} is C1-C6 alkyl.

Embodiment 17. The compound of any one of Embodiments 1-14, wherein R^{1′} is C1-C3 alkyl.

Embodiment 18. The compound of Embodiment 1, wherein R¹ and R^{1′}, together with the carbon atom to which they are attached form a C3-C10 cycloalkyl.

Embodiment 19. The compound of any one of Embodiments 1-18, wherein R² is C6-C12 aryl optionally substituted with 1-3 independently selected R^{2A}.

Embodiment 20. The compound of any one of Embodiments 1-19, wherein R² is phenyl optionally substituted with 1-3 independently selected R^{2A}.

Embodiment 21. The compound of any one of Embodiments 1-19, wherein R² is 2,3-dihydro-1H-indenyl optionally substituted with 1-3 independently selected R^{2A}.

Embodiment 22. The compound of any one of Embodiments 1-18, wherein R² is 5-10 membered heteroaryl optionally substituted with 1-3 independently selected R^{2A}.

Embodiment 23. The compound of any one of Embodiments 1-18, wherein R² is 4-10 membered heterocyclyl optionally substituted with 1-3 independently selected R^{2A}.

Embodiment 24. The compound of any one of Embodiments 1-18, wherein R² is C4-C10 cycloalkyl optionally substituted with 1-3 independently selected R^{2A}.

Embodiment 25. The compound of any one of Embodiments 1-18, wherein R² is C1-C6 aralkyl optionally substituted with 1-4 independently selected R^{2A}.

Embodiment 26. The compound of any one of Embodiments 1-18, wherein R² is C1-C6 aralkyl substituted with 1-4 independently selected R^{2A}.

Embodiment 27. The compound of any one of Embodiments 1-18 or 25, wherein R² is C1-C6 aralkyl.

Embodiment 28. The compound of any one of Embodiments 1-27, wherein 1, 2, or 3 of R^{2A} are independently halogen.

Embodiment 29. The compound of any one of Embodiments 1-28, wherein 1, 2, or 3 of R^{2A} are independently cyano.

Embodiment 30. The compound of any one of Embodiments 1-29, wherein 1, 2, or 3 of R^{2A} are independently hydroxyl.

Embodiment 31. The compound of any one of Embodiments 1-30, wherein 1, 2, or 3 of R^{2A} are independently —NR^{A}R^{B}.

Embodiment 32. The compound of any one of Embodiments 1-31, wherein 1, 2, or 3 of R^{2A} are independently —C(═O)NR^{A}R^{B}.

Embodiment 33. The compound of any one of Embodiments 1-32, wherein 1, 2, or 3 of R^{2A} are independently

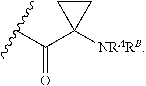

Embodiment 34. The compound of any one of Embodiments 1-33, wherein 1, 2, or 3 of R^{2A} are independently —NHC(═O)R^{C}.

Embodiment 35. The compound of any one of Embodiments 1-34, wherein 1, 2, or 3 of R^{2A} are independently —C(═O)NR^{D}R^{E}.

Embodiment 36. The compound of any one of Embodiments 1-35, wherein 1, 2, or 3 of R^{2A} are independently —C(═O)OR^{F}.

Embodiment 37. The compound of any one of Embodiments 1-36, wherein 1, 2, or 3 of R² are independently —SO₂R^{F}.

Embodiment 38. The compound of any one of Embodiments 1-37, wherein 1, 2, or 3 of R^{2A} are independently —NHSO₂R^{F}.

Embodiment 39. The compound of any one of Embodiments 1-38, wherein 1, 2, or 3 of R^{2A} are independently —SO₂NR^{F}R^{G}.

Embodiment 40. The compound of any one of Embodiments 1-39, wherein 1, 2, or 3 of R^{2A} are independently —NHC(═O)C1-C6 alkyl optionally substituted with NR^{A}R^{B}.

Embodiment 41. The compound of any one of Embodiments 1-40, wherein 1, 2, or 3 of R^{2A} are independently —NHC(═O)C1-C6 alkyl substituted with NR^{A}R^{B}.

Embodiment 42. The compound of any one of Embodiments 1-41, wherein 1, 2, or 3 of R^{2A} are independently C1-C6 haloalkyl.

Embodiment 43. The compound of any one of Embodiments 1-42, wherein 1, 2, or 3 of R^{2A} are independently C1-C6 hydroxyalkyl.

Embodiment 44. The compound of any one of Embodiments 1-43, wherein 1, 2, or 3 of R^{2A} are independently 5-10 membered heteroaryl optionally substituted with 1-3 substituents independently selected from C1-C6 alkyl and —NR^{A}R^{B}.

Embodiment 45. The compound of any one of Embodiments 1-44, wherein 1, 2, or 3 of R^{2A} are independently 5-10 membered heteroaryl substituted with 1-3 substituents independently selected from C1-C6 alkyl and —NR^{A}R^{B}.

Embodiment 46. The compound of any one of Embodiments 1-45, wherein 1, 2, or 3 of R^{2A} are independently 5-10 membered heteroaryl.

Embodiment 47. The compound of any one of Embodiments 1-46, wherein 1, 2, or 3 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

Embodiment 48. The compound of any one of Embodiments 1-47, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

Embodiment 49. The compound of any one of Embodiments 1-48, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}.

Embodiment 50. The compound of any one of Embodiments 1-49, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl.

Embodiment 51. The compound of any one of Embodiments 1-50, wherein 1, 2, or 3 of R^{2A} are independently 4-10 membered heterocyclyl.

Embodiment 52. The compound of any one of Embodiments 1-51, wherein 1, 2, or 3 of R^{2A} are independently C1-C6 alkyl optionally substituted with 1-3 substituents independently selected from hydroxyl, oxo, —NR^{A}R^{B}, —C(═O)NR^{A}R^{B}, C1-C6 alkoxy, C3-C10 cycloalkyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

Embodiment 53. The compound of any one of Embodiments 1-52, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, C3-C10 cycloalkyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

Embodiment 54. The compound of any one of Embodiments 1-53, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, C3-C10 cycloalkyl, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

Embodiment 55. The compound of any one of Embodiments 1-54, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, C3-C10 cycloalkyl, and 4-10 membered heterocyclyl.

Embodiment 56. The compound of any one of Embodiments 1-55, wherein 1, 2, or 3 of R^{2A} are independently C1-C6 alkyl.

Embodiment 57. The compound of any one of Embodiments 1-56, wherein 1, 2, or 3 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

Embodiment 58. The compound of any one of Embodiments 1-57, wherein 1, 2, or 3 of R^{2A} are independently C1-C6 alkoxy substituted with —NR^{A}R^{B} or 4-10 membered heterocyclyl optionally substituted with C1-C6 alkyl, aralkyl, heteroaralkyl, or —C(═O)C3-C6 cycloalkyl.

Embodiment 59. The compound of any one of Embodiments 1-58, wherein 1, 2, or 3 of R^{2A} are independently C1-C6 alkoxy substituted with —NR^{A}R^{B} or 4-10 membered heterocyclyl substituted with C1-C6 alkyl, aralkyl, heteroaralkyl, or —C(═O)C3-C6 cycloalkyl.

Embodiment 60. The compound of any one of Embodiments 1-59, wherein 1, 2, or 3 of R^{2A} are independently C1-C6 alkoxy substituted with —NR^{A}R^{B} or 4-10 membered heterocyclyl.

Embodiment 61. The compound of any one of Embodiments 1-60, wherein 1, 2, or 3 of R^{2A} are independently C1-C6 alkoxy.

Embodiment 62. The compound of any one of Embodiments 1-61, wherein 1, 2, or 3 of R^{2A} are independently C3-C6 cycloalkyl optionally substituted with 4-10 membered heterocyclyl optionally substituted with C1-C6 alkyl.

Embodiment 63. The compound of any one of Embodiments 1-62, wherein 1, 2, or 3 of R^{2A} are independently C3-C6 cycloalkyl substituted with 4-10 membered heterocyclyl optionally substituted with C1-C6 alkyl.

Embodiment 64. The compound of any one of Embodiments 1-63, wherein 1, 2, or 3 of R^{2A} are independently C3-C6 cycloalkyl substituted with 4-10 membered heterocyclyl substituted with C1-C6 alkyl.

Embodiment 65. The compound of any one of Embodiments 1-64, wherein 1, 2, or 3 of R^{2A} are independently C3-C6 cycloalkyl substituted with 4-10 membered heterocyclyl.

Embodiment 66. The compound of any one of Embodiments 1-58, wherein 1, 2, or 3 of R^{2A} are independently C3-C6 cycloalkyl.

Embodiment 67. The compound of any one of Embodiments 1-18, wherein R² is C1-C6 alkoxy optionally substituted with —C(═O)NR^{A}R^{C}.

Embodiment 68. The compound of any one of Embodiments 1-18 or 67, wherein R² is C1-C6 alkoxy substituted with —C(═O)NR^{A}R^{C}.

Embodiment 69. The compound of any one of Embodiments 1-18 or 67, wherein R² is C1-C6 alkoxy.

Embodiment 70. The compound of any one of Embodiments 1-18, wherein R² is C1-C6 alkoxyalkyl optionally substituted with —C(═O)NR^{A}R^{C}.

Embodiment 71. The compound of any one of Embodiments 1-18, wherein R² is C1-C6 alkoxyalkyl.

Embodiment 72. The compound of any one of Embodiments 1-71, wherein X is a bond.

Embodiment 73. The compound of any one of Embodiments 1-71, wherein X is CH₂.

Embodiment 74. The compound of any one of Embodiments 1-71, wherein X is CH(CH₃).

Embodiment 75. The compound of any one of Embodiments 1-71, wherein X is C(CH₃)₂.

Embodiment 76. The compound of any one of Embodiments 1-71, wherein X is

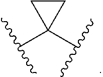 [SMILES: *C1(*)CC1]

Embodiment 77. The compound of any one of Embodiments 1-76, wherein one of R^{3A} and R^{3B} is hydrogen and the other of R^{3A} and R^{3B} is C1-C6 alkyl.

Embodiment 78. The compound of any one of Embodiments 1-77, wherein one of R^{3A} and R^{3B} is hydrogen and the other of R^{3A} and R^{3B} is methyl.

Embodiment 79. The compound of any one of Embodiments 1-76, wherein each of R^{3A} and R^{3B} is hydrogen.

Embodiment 80. The compound of any one of Embodiments 1-76, wherein each of R^{3A} and R^{3B} is an independently selected C1-C6 alkyl.

Embodiment 81. The compound of any one of Embodiments 1-76 or 80, wherein each of R^{3A} and R^{3B} is methyl.

Embodiment 82. The compound of any one of Embodiments 1-76, wherein one of R^{3A} and R^{3B} is hydrogen and the other of R^{3A} and R^{3B} is C1-C6 alkoxy.

Embodiment 83. The compound of any one of Embodiments 1-76, wherein one of R^{3A} and R^{3B} is C1-C6 alkyl and the other of R^{3A} and R^{3B} is C1-C6 alkoxy.

Embodiment 84. The compound of any one of Embodiments 1-76, wherein each of R^{3A} and R^{3B} is C1-C6 alkoxy.

Embodiment 85. The compound of any one of Embodiments 1-76, wherein one of R^{3A} and R^{3B} is hydrogen and the other of R^{3A} and R^{3B} is C1-C6 haloalkyl.

Embodiment 86. The compound of any one of Embodiments 1-76, wherein one of R^{3A} and R^{3B} is C1-C6 alkyl and the other of R^{3A} and R^{3B} is C1-C6 haloalkyl.

Embodiment 87. The compound of any one of Embodiments 1-76, wherein each of R^{3A} and R^{3B} is C1-C6 haloalkyl.

Embodiment 88. The compound of any one of Embodiments 1-76, wherein R^{3A} and R^{3B}, together with the carbon and nitrogen atoms, respectively, to which they are attached together form a 4-8 membered heterocyclyl group.

Embodiment 89. The compound of any one of Embodiments 1-88, wherein Y is phenyl optionally substituted with 1-3 independently selected R^{Y}.

Embodiment 90. The compound of any one of Embodiments 1-88, wherein Y is naphthyl optionally substituted with 1-3 independently selected R^{Y}.

Embodiment 91. The compound of any one of Embodiments 1-88, wherein Y is 5-10 membered heteroaryl optionally substituted with 1-3 independently selected R^{Y}.

Embodiment 92. The compound of any one of Embodiments 1-91, wherein 1, 2, or 3 of R^{Y} is independently halogen.

Embodiment 93. The compound of any one of Embodiments 1-92, wherein 1, 2, or 3 of R^{Y} is hydroxyl.

Embodiment 94. The compound of any one of Embodiments 1-93, wherein 1, 2, or 3 of R^{Y} is cyano.

Embodiment 95. The compound of any one of Embodiments 1-94, wherein 1, 2, or 3 of R^{Y} is independently C1-C6 haloalkyl.

Embodiment 96. The compound of any one of Embodiments 1-95, wherein 1, 2, or 3 of R^{Y} is independently C1-C6 alkoxy.

Embodiment 97. The compound of any one of Embodiments 1-96, wherein 1, 2, or 3 of R^{Y} is independently C1-C6 haloalkoxy.

Embodiment 98. The compound of any one of Embodiments 1-97, wherein 1, 2, or 3 of R^{Y} is independently C1-C6 hydroxyalkyl.

Embodiment 99. The compound of any one of Embodiments 1-98, wherein 1, 2, or 3 of R^{Y} is independently —NHC(═O)R^{C}.

Embodiment 100. The compound of any one of Embodiments 1-99, wherein 1, 2, or 3 of R^{Y} is independently —C(═O)NHR^{Y1}.

Embodiment 101. The compound of any one of Embodiments 1-100, wherein 1, 2, or 3 of R^{Y} is independently —CO₂R^{A}.

Embodiment 102. The compound of any one of Embodiments 1-101, wherein 1, 2, or 3 of R^{Y} is independently —SO₂NR^{F}R^{G}.

Embodiment 103. The compound of any one of Embodiments 1-102, wherein 1, 2, or 3 of R^{Y} is independently —NHSO₂R^{F}.

Embodiment 104. The compound of any one of Embodiments 1-103, wherein 1, 2, or 3 of R^{Y} is independently —S(═O)(═NR^{F})R^{G}.

Embodiment 105. The compound of any one of Embodiments 1-104, wherein 1, 2, or 3 of R^{Y} is independently —SO₂(C1-C6 alkyl).

Embodiment 106. The compound of any one of Embodiments 1-105, wherein 1, 2, or 3 of R^{Y} is independently —C(═O)NR^{A}R^{B}.

Embodiment 107. The compound of any one of Embodiments 1-106, wherein 1, 2, or 3 of R^{Y} is independently 5-6 membered heteroaryl.

Embodiment 108. The compound of any one of Embodiments 1-107, wherein 1, 2, or 3 of R^{Y} is independently heteroaralkyl.

Embodiment 109. The compound of any one of Embodiments 1-108, wherein 1, 2, or 3 of R^{Y} is independently C1-C6 alkyl optionally substituted with —CO₂R^{A} or 5-6 membered heteroaryl optionally substituted with R^{Y1}.

Embodiment 110. The compound of any one of Embodiments 1-109, wherein 1, 2, or 3 of R^{Y} is independently C1-C6 alkyl substituted with —CO₂R^{A} or 5-6 membered heteroaryl optionally substituted with R^{Y1}.

Embodiment 111. The compound of any one of Embodiments 1-110, wherein 1, 2, or 3 of R^{Y} is independently C1-C6 alkyl substituted with —CO₂R^{A} or 5-6 membered heteroaryl substituted with R^{Y1}.

Embodiment 112. The compound of any one of Embodiments 1-109, wherein 1, 2, or 3 of R^{Y} is independently C1-C6 alkyl substituted with —CO₂R^{A} or 5-6 membered heteroaryl.

Embodiment 113. The compound of any one of Embodiments 1-109, wherein 1, 2, or 3 of R^{Y} is independently C1-C6 alkyl.

Embodiment 114. The compound of any one of Embodiments 1-91 or 109-111, wherein R^{Y1} is —SO₂(C1-C6 alkyl).

Embodiment 115. The compound of any one of Embodiments 1-91 or 109-111, wherein R^{Y1} is C1-C6 alkyl optionally substituted with oxo.

Embodiment 116. The compound of any one of Embodiments 1-115, wherein R⁴ is hydrogen.

Embodiment 117. The compound of any one of Embodiments 1-115, wherein R⁴ is C1-C6 alkyl.

Embodiment 118. The compound of any one of Embodiments 1-115, wherein R⁴ is acrylamido.

Embodiment 119. The compound of any one of Embodiments 1-118, wherein R⁵ is hydrogen.

Embodiment 120. The compound of any one of Embodiments 1-118, wherein R⁵ is C1-C6 alkyl.

Embodiment 121. The compound of any one of Embodiments 1-118, wherein R⁵ is halogen.

Embodiment 122. The compound of any one of Embodiments 1-118, wherein R⁵ is C1-C6 haloalkyl.

Embodiment 123. The compound of any one of Embodiments 1-118, wherein R⁵ is C3-C6 cycloalkyl.

Embodiment 124. The compound of any one of Embodiments 1-118, wherein R⁵ is cyano.

Embodiment 125. The compound of any one of Embodiments 1-118, wherein R⁵ is —NR^{5A}R^{5B}.

Embodiment 126. The compound of any one of Embodiments 1-118, wherein R⁵ is —C(═O)NR^{5A}R^{5B}.

Embodiment 127. The compound of any one of Embodiments 1-118, wherein R⁵ is —NR^{5A}C(═O)R^{5B}.

Embodiment 128. The compound of any one of Embodiments 1-118 or 125-127, wherein one of R^{5A} and R^{5B} is hydrogen and the other of R^{5A} and R^{5B} is C1-C6 alkyl, C2-C6 alkenyl, or C1-C6 hydroxyalkyl.

Embodiment 129. The compound of any one of Embodiments 1-118 or 125-127, wherein one of R^{5A} and R^{5B} is C1-C6 alkyl and the other of R^{5A} and R^{5B} is C1-C6 alkyl, C2-C6 alkenyl, or C1-C6 hydroxyalkyl.

Embodiment 130. The compound of any one of Embodiments 1-118 or 125-127, wherein each of R^{5A} and R^{5B} is hydrogen.

Embodiment 131. The compound of any one of Embodiments 1-118 or 125-127, wherein each of R^{5A} and R^{5B} is an independently selected C1-C6 alkyl.

Embodiment 132. The compound of any one of Embodiments 1-131, wherein R⁶ is hydrogen.

Embodiment 133. The compound of any one of Embodiments 1-131, wherein R⁶ is halogen.

Embodiment 134. The compound of any one of Embodiments 1-131, wherein R⁶ is C1-C6 alkyl.

Embodiment 135. The compound of any one of Embodiments 1-134, wherein each of R^{A} and R^{B} are independently selected from hydrogen, hydroxyl, C1-C6 alkoxy, C3-C6 cycloalkyl, C2-C6 alkenyl, cyano, and C1-C6 alkyl optionally substituted with hydroxyl or C1-C6 alkoxy.

Embodiment 136. The compound of any one of Embodiments 1-135, wherein one of R^{A} and R^{B} is hydrogen and the other of R^{A} and R^{B} is hydroxyl, C1-C6 alkoxy, C3-C6 cycloalkyl, C2-C6 alkenyl, cyano, or C1-C6 alkyl optionally substituted with hydroxyl or C1-C6 alkoxy.

Embodiment 137. The compound of any one of Embodiments 1-136, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

Embodiment 138. The compound of any one of Embodiments 1-136, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

Embodiment 139. The compound of any one of Embodiments 1-134, wherein R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl.

Embodiment 140. The compound of any one of Embodiments 1-134 or 139, wherein R^{A} and R^{B} together with the nitrogen atom to which they are attached form a 4-10 membered heterocyclyl substituted with 1-2 substituents independently selected from halogen, C1-C6 alkyl, and —C(═O)C1-C6 alkyl.

Embodiment 141. The compound of any one of Embodiments 1-134 or 139, wherein R^{A} and R^{B} together with the nitrogen atom to which they are attached form a 4-10 membered heterocyclyl.

Embodiment 142. The compound of any one of Embodiments 1-141, wherein each R^{C} is independently C3-C6 cycloalkyl, —C(═O)NHR^{Y1}, or a C1-C6 alkyl substituted with —NR^{A}R^{B} or with 4-10 membered heterocyclyl optionally substituted with C1-C6 alkyl or with C1-C6 hydroxylalkyl.

Embodiment 143. The compound of any one of Embodiments 1-142, wherein each R^{C} is independently C3-C6 cycloalkyl, —C(═O)NHR^{Y1}, or a C1-C6 alkyl substituted with —NR^{A}R^{B} or with 4-10 membered heterocyclyl substituted with C1-C6 alkyl or with C1-C6 hydroxylalkyl.

Embodiment 144. The compound of any one of Embodiments 1-142, wherein each R^{C} is independently C3-C6 cycloalkyl, —C(═O)NHR^{Y1}, or a C1-C6 alkyl substituted with —NR^{A}R^{B} or with 4-10 membered heterocyclyl.

Embodiment 145. The compound of any one of Embodiments 1-142, wherein each R^{C} is independently C3-C6 cycloalkyl, —C(═O)NHR^{Y1}, or a C1-C6 alkyl.

Embodiment 146. The compound of any one of Embodiments 1-145, wherein each R^{D} is hydrogen, hydroxyl, or C1-C6 alkoxy.

Embodiment 147. The compound of any one of Embodiments 1-145, wherein each R^{D} is C1-C6 alkyl, 4-10 membered heterocyclyl, or phenyl.

Embodiment 148. The compound of any one of embodiments 1-145, wherein each R^{D} is C1-C6 alkyl, 4-10 membered heterocyclyl, or phenyl optionally substituted with 4-10 membered heterocyclyl, C3-C6 cycloalkyl, or cyano.

Embodiment 149. The compound of any one of embodiments 1-145, wherein each R^{D} is C3-C6 cycloalkyl.

Embodiment 150. The compound of any one of embodiments 1-145, wherein each R^{D} is C3-C6 cycloalkyl optionally substituted with C1-C6 alkyl optionally substituted with hydroxyl or C1-C6 alkoxy.

Embodiment 151. compound of any one of embodiments 1-150, wherein each R^{E} is hydrogen, hydroxyl, or C1-C6 alkoxy.

Embodiment 152. The compound of any one of embodiments 1-150, wherein each R^{E} is C1-C6 alkyl, 4-10 membered heterocyclyl, or phenyl.

Embodiment 153. The compound of any one of embodiments 1-150, wherein each R^{E} is C1-C6 alkyl, 4-10 membered heterocyclyl, or phenyl optionally substituted with 4-10 membered heterocyclyl, C3-C6 cycloalkyl, or cyano.

Embodiment 154. The compound of any one of embodiments 1-150, wherein each R^{E} is C3-C6 cycloalkyl.

Embodiment 155. The compound of any one of embodiments 1-150, wherein each R^{E} is C3-C6 cycloalkyl optionally substituted with C1-C6 alkyl optionally substituted with hydroxyl or C1-C6 alkoxy.

Embodiment 156. The compound of any one of Embodiments 1-145, wherein one of R^{D} and R^{E} is hydrogen and the other of R^{D} and R^{E} is hydroxyl, C1-C6 alkyl, or C1-C6 alkoxy.

Embodiment 157. The compound of any one of Embodiments 1-145, wherein each of R^{D} and R^{E} is hydrogen.

Embodiment 158. The compound of any one of Embodiments 1-145, wherein each of R^{D} and R^{E} is an independently selected C1-C6 alkyl.

Embodiment 159. The compound of any one of Embodiments 1-158, wherein one of R^{E} and R^{G} is hydrogen and the other of R^{E} and R^{G} is phenyl or C1-C6 alkyl optionally substituted with oxo or —NR^{A}R^{B}.

Embodiment 160. The compound of any one of Embodiments 1-158, wherein one of R^{F} and R^{G} is hydrogen and the other of R^{F} and R^{G} is phenyl or C1-C6 alkyl substituted with oxo or —NR^{A}R^{B}.

Embodiment 161. The compound of any one of Embodiments 1-158, wherein one of R^{F} and R^{G} is hydrogen and the other of R^{F} and R^{G} is phenyl or C1-C6 alkyl.

Embodiment 162. The compound of any one of Embodiments 1-158, wherein each of R^{F} and R^{G} is hydrogen.

Embodiment 163. The compound of any one of Embodiments 1-158, wherein each of R^{F} and R^{G} is an independently selected C1-C6 alkyl.

Embodiment 164. The compound of Embodiment 1, wherein, the compound of Formula (I), or a pharmaceutically acceptable salt thereof, is a compound of Formula (I-A2):

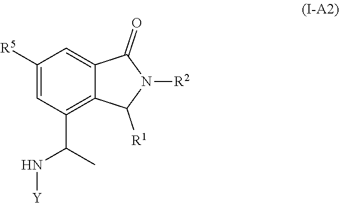

or a pharmaceutically acceptable salt thereof.

Embodiment 165. A compound selected from the group consisting of the compounds in Table A, or a pharmaceutically acceptable salt thereof.

Embodiment 166. A pharmaceutical composition comprising a compound of any one of Embodiments 1-165, or a pharmaceutically acceptable salt thereof, and one or more pharmaceutically acceptable excipients.

Embodiment 167. A method for treating cancer in a subject in need thereof, comprising administering to the subject a therapeutically effective amount of a compound of any one of Embodiments 1-165, or a pharmaceutically acceptable salt thereof, or a pharmaceutical composition of Embodiment 166.

Embodiment 168. A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of any one of Embodiments 1-165, or a pharmaceutically acceptable salt thereof, or a pharmaceutical composition of Embodiment 166.

Embodiment 169. A method of treating a PI3Kα-associated cancer in a subject, comprising administering to a subject identified or diagnosed as having a PI3Kα-associated cancer a therapeutically effective amount of a compound of any one of Embodiments 1-165 or a pharmaceutically acceptable salt thereof, or a pharmaceutical composition of Embodiment 166.

Embodiment 170. A method for inhibiting mutant PI3Kα activity in a mammalian cell, the method comprising contacting the mammalian cell with an effective amount of a compound of any one of Embodiments 1-165, or a pharmaceutically acceptable salt thereof.

What is claimed is:

1. A compound of Formula (I): 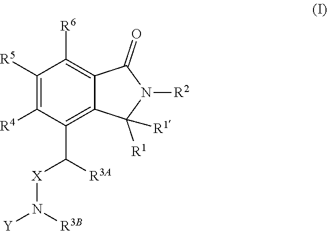
or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1′} is hydrogen or C1-C6 alkyl; or R¹ and R^{1′}, together with the carbon atom to which they are attached form a C3-C10 cycloalkyl;
R² is C6-C12 aryl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, C4-C10 cycloalkyl optionally substituted with 1-4 independently selected R^{2A}, C1-C6 alkoxyalkyl optionally substituted with —C(═O)NR^{A}R^{C}, C1-C6 aralkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B}, 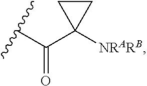
(vii) —NHC(═O)R^{C},
(viii) —C(═O)R^{D},
(ix) —C(═O)OR^{E},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl, —C(═O)NR^{A}R^{B}, C1-C6 haloalkyl, and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, C3-C10 cycloalkyl, phenyl, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, hydroxyl, aralkyl, heteroaralkyl, or —C(═O)C3-C6 cycloalkyl,
(xx) C3-C6 cycloalkyl optionally substituted with 4-10 membered heterocyclyl optionally substituted with C1-C6 alkyl;
(xxi) C6-C12 aryl optionally substituted with 1-3 substituents independently selected from hydroxyl, cyano, C1-C6 haloalkyl, —OR^{E}, —C(═O)C1-C6 alkyl, —SO₂(C1-C6 alkyl), —SO₂NR^{F}R^{G}, 4-10 membered heterocyclyl, or C1-C6 alkyl optionally substituted with C1-C6 alkoxy, hydroxyl, or —C(═O)NR^{A}R^{B}; and
(xxii) 4-10 membered heterocyclyloxy optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C6-C12 aryl, C1-C6 alkyl optionally substituted with C1-C6 alkoxy, —C(═O)NR^{A}R^{B}, or —NHC(═O)C1-C6 alkyl optionally substituted with —NR^{A}R^{B};
each R^{A} and R^{B} is independently selected from hydrogen, hydroxyl, C1-C6 alkoxy, C3-C6 cycloalkyl, C2-C6 alkenyl, cyano, 4-10 membered heterocyclyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, hydroxyl, C1-C6 alkyl, and —C(═O)C1-C6 alkyl;
each R^{C} is independently selected from C3-C6 cycloalkyl, —C(═O)NHR^{Y1}, C2-C6 alkenyl, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, phenyl, 4-10 membered heterocyclyl, —NR^{A}R^{B}, C1-C6 alkoxy, and C3-C6 cycloalkyl, wherein the C1-C6 alkyl, 4-10 membered heterocyclyl, and phenyl are optionally substituted with 4-10 membered heterocyclyl, C3-C6 cycloalkyl, or cyano, and the C3-C6 cycloalkyl is optionally substituted with C1-C6 alkyl optionally substituted with hydroxyl or C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, halogen, C1-C6 haloalkyl, C3-C6 cycloalkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or 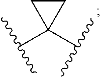 [SMILES: *C1(*)CC1]
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

2. The compound of claim 1, wherein R² is C6-C12 aryl optionally substituted with 1-3 independently selected R^{2A}.

3. The compound of claim 1, wherein R² is phenyl optionally substituted with 1-3 independently selected R^{2A}.

4. The compound of claim 1, wherein R² is 5-10 membered heteroaryl optionally substituted with 1-3 independently selected R^{2A}.

5. The compound of claim 1, wherein R² is 4-10 membered heterocyclyl optionally substituted with 1-3 independently selected R^{2A}.

6. The compound of claim 1, wherein R² is C4-C10 cycloalkyl optionally substituted with 1-3 independently selected R^{2A}.

7. The compound of any one of claims 1-6, wherein 1, 2, or 3 of R^{2A} are independently halogen.

8. The compound of any one of claims 1-7, wherein 1, 2, or 3 of R^{2A} are independently cyano.

9. The compound of any one of claims 1-8, wherein 1, 2, or 3 of R^{2A} are independently hydroxyl.

10. The compound of any one of claims 1-9, wherein 1, 2, or 3 of R^{2A} are independently C1-C6 haloalkyl.

11. The compound of any one of claims 1-10, wherein 1, 2, or 3 of R^{2A} are independently C1-C6 hydroxyalkyl.

12. The compound of any one of claims 1-11, wherein 1, 2, or 3 of R^{2A} are independently 5-10 membered heteroaryl optionally substituted with 1-3 substituents independently selected from C1-C6 alkyl and —NR^{A}R^{B}.

13. The compound of any one of claims 1-12, wherein 1, 2, or 3 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

14. The compound of any one of claims 1-13, wherein 1, 2, or 3 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

15. The compound of any one of claims 1-14, wherein 1, 2, or 3 of R^{2A} are independently C3-C6 cycloalkyl optionally substituted with 4-10 membered heterocyclyl optionally substituted with C1-C6 alkyl.

16. The compound of any one of claims 1-15, wherein X is a bond.

17. The compound of any one of claims 1-16, wherein each of R^{3A} and R^{3B} is hydrogen.

18. The compound of any one of claims 1-17, wherein Y is phenyl optionally substituted with 1-3 independently selected R^{Y}.

19. The compound of any one of claims 1-17, wherein Y is 5-10 membered heteroaryl optionally substituted with 1-3 independently selected R^{Y}.

20. The compound of any one of claims 1-19, wherein 1, 2, or 3 of R is independently —CO₂R^{A}.

21. The compound of claim 1, wherein, the compound of Formula (I), or a pharmaceutically acceptable salt thereof, is a compound of Formula (I-A2): 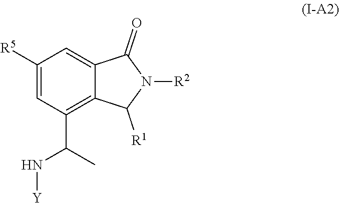
or a pharmaceutically acceptable salt thereof.

22. A compound selected from the group consisting of the compounds in Table A, or a pharmaceutically acceptable salt thereof.

23. A pharmaceutical composition comprising a compound of any one of claims 1-22, or a pharmaceutically acceptable salt thereof, and one or more pharmaceutically acceptable excipients.

24. A method for treating cancer in a subject in need thereof, comprising administering to the subject a therapeutically effective amount of a compound of any one of claims 1-22, or a pharmaceutically acceptable salt thereof, or a pharmaceutical composition of claim 23.

25. A method of treating a PI3Kα-associated cancer in a subject, comprising administering to a subject identified or diagnosed as having a PI3Kα-associated cancer a therapeutically effective amount of a compound of any one of claims 1-22 or a pharmaceutically acceptable salt thereof, or a pharmaceutical composition of claim 23.
